(19) United States
(12) Patent Application Publication
(10) Pub. No.: US 2007/0264643 A1
(43) Pub. Date: Nov. 15, 2007

(54) Compositions and Methods Relating to CNS Lymphoma

(75) Inventors: James Rubenstein, Palo Alto, CA (US); Howard Schulman, Palo Alto, CA (US); Christopher H. Becker, Palo Alto, CA (US); Shushmita Mimi Roy, Sunnyvale, CA (US)

Correspondence Address: DON D. CHA, 17225 W. 12TH AVE., GOLDEN, CO, 80401, US

(73) Assignee: PPD BIOMARKER SERVICES, INC.

(21) Appl. No.: 11/551,810

(22) Filed: Oct. 23, 2006

Related U.S. Application Data
(63) Continuation of application No. 11/364,350, filed on Feb. 27, 2006, now abandoned.
(60) Provisional application No. 60/656,749, filed on Feb. 25, 2005.

(51) Int. Cl. C12Q 1/68; G01N 33/53

(52) U.S. Cl. 435/6; 435/7.1

(57) ABSTRACT

Compositions, methods and kits useful for the diagnosis, prognosis, and treatment of CNS lymphoma.

### CROSS-REFERENCE TO RELATED APPLICATIONS

This application is a continuation of U.S. patent application Ser. No. 11/364,350, filed Feb. 27, 2006, which claims the benefit of priority under 35 U.S.C. § 119(e) from U.S. Provisional Application Ser. No. 60/656,749, filed Feb. 25, 2005. The entire disclosure of U.S. Provisional Application Ser. No. 60/656,749 is incorporated herein by reference.

### FIELD OF THE INVENTION

The present invention provides compositions, methods and kits useful for the diagnosis, prognosis, and treatment of central nervous system (CNS) lymphoma. In particular, the invention provides polypeptides that are markers of CNS lymphoma, polynucleotides that encode the polypeptides and antibodies and aptamers that specifically bind to the polypeptides. The invention also provides fragments, precursors, successors and modified versions of the foregoing polypeptides, polynucleotides, antibodies and aptamers.

The invention also provides compositions comprising the foregoing polypeptides, polynucleotides, antibodies, and aptamers. The invention also provides methods for using the polypeptides, polynucleotides, aptamers and antibodies in the diagnosis and treatment of CNS lymphoma, monitoring progression of the disease and screening of candidate therapeutic compounds.

### BACKGROUND OF THE INVENTION

The non-Hodgkin's lymphomas (NHL) represent a major cause of cancer-related morbidity and death. Approximately 56,000 new cases of non-Hodgkin's lymphoma occur each year in the U.S., resulting in over 25,000 deaths annually. Moreover, the incidence of NHL is increasing at a rate of approximately 4% per year. CNS involvement of NHL is associated with an adverse prognosis and can occur by two pathways: primary CNS lymphoma and secondary dissemination of systemic lymphoma to the brain. The vast majority of lymphomas that involve the CNS are large, B cell neoplasms which express CD20 (Fine et al., Ann. Intern. Med. 119:1093-1104, 1993).

In a recent prospective analysis, CNS complications of systemic NHL were identified as the main cause of death in a group of 606 newly diagnosed patients with immunoblastic or large cell lymphoma who had received adequate systemic treatment (Van Besien et al., Blood 91:1174-1184, 1998). Dissemination within the leptomeninges represents a common pathway for progression of both systemic NHL and for primary CNS lymphoma (Chamberlain et al., Oncology Reports 5:521-525, 1998).

Prior methods of diagnosis of brain and leptomeningeal metastases involving lymphoma are unsafe or unreliable. Stereotactic brain biopsy represents an important technique for the histologic evaluation of intracranial tumors. This procedure, however, is associated with significant risk because patients require general anesthesia, endure placement of a twist-drill or burr hole for skull penetration and encounter a 1.2% to 7% risk of severe complications which include catastrophic intracranial hemorrhage. Not all patients with presumptive brain metastases are good candidates for this procedure because of patient age, comorbid medical illness, the location of the lesion in eloquent or deep brain structures, or in tumors that are highly vascularized. Stereotactic biopsy is associated with an 8-9% failure rate, defined as a biopsy in which a definitive histological diagnosis is not achieved based on the tissue obtained (Bernstein et al., Neuro-oncology: The Essentials, Thieme Medical Publishers, 2000).

Less invasive diagnostic methods include magnetic resonance imaging (MRI) and cerebrospinal fluid evaluation. Unfortunately, gadolinium-enhanced MRI only detects tumor-associated contrast enhancement of at least 0.5 cm in diameter. Thus, it lacks specificity in the evaluation of brain tumors. Imaging by MRI of the neuroaxis in evaluating leptomeningeal disease is even less sensitive and is associated with at least 30%-50% false negatives (Chamberlain, Curr Opin Neurol 13:641-648, 2000). Cytologic evaluation of cerebrospinal fluid (CSF) only requires lumbar puncture which is a routine procedure. Although the procedure is safe and much less invasive than a brain biopsy, the cytological analysis of CSF is insensitive. Between 40%-50% of patients with neoplastic meningitis have negative CSF cytology (Chamberlain, ibid.). It has also been shown that CSF cytology has almost no value in the diagnosis of parenchymal brain lesions.

Thus, there is an unmet medical need for a safe and reliable diagnostic for detection of primary CNS lymphoma and dissemination of lymphoma to the CNS.

### SUMMARY OF THE INVENTION

One aspect of the invention provides polypeptides (“polypeptide markers”) that have been identified as differentially expressed in CNS lymphoma samples, including CSF samples from patients with CNS lymphoma, as compared to CSF samples obtained from control patients without cancer. The invention also provides polypeptides that have been identified as differentially expressed in patients with a CNS cancer, including CSF lymphoma and metastatic brain cancers. The invention also provides polypeptides that have substantial sequence identity to polypeptide markers, modified polypeptide markers, and fragments of the polypeptide markers. The invention also includes precursors and successors of the polypeptide markers in biological pathways. The invention also provides molecules that comprise a polypeptide marker, homologous polypeptides, a modified polypeptide marker or a fragment thereof, precursor or successor of a polypeptide marker (e.g., a fusion protein). As used herein, the term “polypeptides of the invention” shall be understood to include all of the foregoing.

The invention also provides polypeptides that have been identified as differentially expressed in cerebrospinal fluid (CSF) samples, including samples obtained from patients with CNS lymphoma as compared to CSF samples obtained from patients that do not have CNS lymphoma. In some embodiments, the marker is a polypeptide comprising a marker identified in Tables 1-7, or a polynucleotide encoding a polypeptide comprising a marker identified in Tables 1-7. In other embodiments, the marker is Antithrombin III, Complement Factor H, or EGF-containing fibulin-like extracellular matrix protein 1 (EFEMP1), also known as Fibulin-3 (FBLN3). In other embodiments, the marker is a member of the Fibulin family. Another aspect of the invention provides polynucleotides encoding polypeptides of the invention (“polynucleotide markers”). The invention also provides polynucleotides that have substantial sequence identity to polynucleotide markers, modified polynucleotide markers, and fragments of polynucleotide markers. The invention also provides molecules that comprise a polynucleotide marker, a homologous polynucleotide, a modified polynucleotide marker or a fragment of a polynucleotide marker (e.g., a vector). As used herein, the term “polynucleotides of the invention” shall be understood to include all of the foregoing.

Another aspect of the invention provides molecules that specifically bind to a polypeptide of the invention or polynucleotide of the invention. The binding molecule may be an antibody, antibody fragment, apatmer, or other molecule. The invention also provides methods for producing a binding molecule that specifically recognizes a polypeptide of the invention or polynucleotide of the invention.

Another aspect of the invention provides compositions comprising a polypeptide of the invention or polynucleotide of the invention, a binding molecule (e.g., an antibody or aptamer) that is specific for a polypeptide of the invention or polypeptide of the invention, an inhibitor of a polypeptide of the invention or polynucleotide of the invention, or another molecule that can increase or decrease the level or activity of a polypeptide of the invention or polynucleotide of the invention. Such compositions may be pharmaceutical compositions formulated for use as therapeutics.

Another aspect of the invention provides a method for detecting a polypeptide of the invention or polynucleotide of the invention. In one embodiment, the method comprises contacting a biological sample obtained from a subject with a binding molecule (e.g., an antibody or aptamer) under conditions that permit the formation of a stable complex, and detecting any stable complexes formed. In another embodiment, the method comprises determining the activity of a polypeptide of the invention or polynucleotide of the invention. In another embodiment, the method comprises determining the level of a polypeptide of the invention in a cell obtained from the subject by detecting the presence of a polynucleotide that encodes the polypeptide.

Another aspect of the invention provides a method for diagnosing CNS lymphoma in a subject by detecting a polypeptide of the invention or polynucleotide of the invention in a biological sample. In one embodiment, the method comprises obtaining a sample from a subject suspected of having CNS lymphoma or at risk for CNS lymphoma and comparing the level or activity of a polypeptide of the invention or polynucleotide of the invention in the sample with the level of activity in a sample obtained from a non-CNS lymphoma subject or with a reference range or value. In some embodiments, CNS lymphoma is diagnosed in the patient if the expression level of the biomarker or biomarkers in the patient sample is statistically more similar to the expression level of the biomarker or biomarkers that has been associated with CNS lymphoma than the expression level of the biomarker or biomarkers that has been associated with the normal controls. In some embodiments, the method is used for staging or stratifying subjects with CNS lymphoma, monitoring the progression of the disease or response to therapy. In some embodiments, a plurality of polypeptides of the invention or polynucleotides of the invention are detected. In some embodiments, the method comprises detecting known biomarkers or considering other clinical indicia in addition to detecting one or more polypeptides of the invention or polynucleotides of the invention in a biological sample.

Another aspect of the invention provides methods for treating CNS lymphoma by administering a therapeutic agent to a subject that increases or decreases the level or activity of a polypeptide of the invention or polynucleotide of the invention. For polypeptides of the invention or polynucleotides of the invention that are increased in samples obtained from a CNS lymphoma subject, the method comprises administering a therapeutic agent that decreases (i.e., bring toward the normal range) the level or activity of the polypeptide or polynucleotide. Similarly, for polypeptides of the invention or polynucleotides of the invention that are decreased in samples obtained from a CNS lymphoma subject, the method comprises administering a therapeutic agent that increases the level or activity of the polypeptide or polynucleotide.

Another aspect of the present invention provides a method for screening a candidate compound for use as a therapeutic agent for treating CNS lymphoma. In one embodiment, the method comprises administering the candidate compound to a CNS lymphoma subject and screening for the ability to modulate the level or activity of a polypeptide of the invention or polynucleotide of the invention. In another embodiment, the method comprises providing the candidate compound to a cell from a CNS lymphoma subject and screening for the ability to modulate the intracellular level of a polypeptide of the invention or polynucleotide of the invention.

Another aspect of the invention provides a kit for performing the methods described above. In one embodiment, the kit is for the diagnosis of CNS lymphoma by detection of a polypeptide of the invention or polynucleotide of the invention in a biological sample from a subject. A kit for detecting a polypeptide of the invention or polynucleotide of the present invention may include an antibody or aptamer capable of binding to the polypeptide or polynucleotide.

Another aspect of the invention includes the use of animal models of CNS lymphoma. For example, the markers identified in the present application can be used in research aimed to discover and/or test biomarkers with relevance in humans.

Other features and advantages of the invention will become apparent to one of skill in the art from the following detailed description, including Tables 1-7, the drawings, and from the claims.

### BRIEF DESCRIPTION OF THE DRAWINGS

FIG. 1 shows a validation of Antithrombin III as a marker in CNS lymphoma via a Western blot in CSF samples from various patients. Lane A: benign; multiple sclerosis; Lane B: benign; multiple sclerosis; Lane C: benign; neurosarcoid; Lane D: benign; Lane E: benign; Lane F: CNS lymphoma; Lane G: CNS lymphoma; Lane H: CNS lymphoma; Lane I: CNS lymphoma; Lane J: CNS lymphoma; Lane K: CNS lymphoma.

FIG. 2A shows the concentration of marker Antithrombin III in normal and CSF lymphoma patients.

FIG. 2B shows the specificity/sensitivity of Antithrombin III through an ROC curve (AUC=0.85).

FIG. 3 shows survival over time for CSF lymphoma patients with low Antithrombin III levels vs. high Antithrombin III levels.

FIG. 4 shows the presence of EFEMP1 (EGF-containing fibulin-like extracellular matrix protein 1 (EFEMP1), also known as Fibulin-3 (FBLN3)) via a Western blot in CSF samples from various patients. Lane A: benign; multiple sclerosis; Lane B: benign; multiple sclerosis; Lane C: benign; neurosarcoid; Lane D: benign; Lane E: benign; Lane F: CNS lymphoma; Lane G: CNS lymphoma; Lane H: CNS lymphoma; Lane I: CNS lymphoma; Lane J: CNS lymphoma; Lane K: CNS lymphoma; Lane L: CNS lymphoma.

FIG. 5 shows the presence of Complement Factor H via a Western blot in CSF samples from various patients. Lane A: benign; multiple sclerosis; Lane B: benign; multiple sclerosis; Lane C: benign; neurosarcoid; Lane D: benign; Lane E: benign; Lane F: CNS lymphoma; Lane G: CNS lymphoma; Lane H: CNS lymphoma; Lane I: CNS lymphoma; Lane J: CNS lymphoma; Lane K: CNS lymphoma; Lane L: CNS lymphoma; Lane M; recombinant peptide.

FIG. 6A-D shows MRI results from a patient with CNS lymphoma. FIG. 6A shows the patient who was MRI positive and had positive CSF cytology. FIG. 6B is a later time point at which the patient had recurrent leptomeningeal CNS Lymphoma after intrathecal therapy, where the patient had positive CSF cytology. FIG. 6C shows progression of disease at a later time point with new brain parenchymal involvement, and persistent positive CSF cytology. FIG. 6D shows negative MRI at a later time point after high-dose systemic methotrexate chemotherapy, where the patient had negative CSF cytology.

FIG. 7 shows serial measurement of Antithrombin III by ELISA in one patient. CNS lymphoma progression and therapeutic response are reflected by the rise and fall in CSF concentrations of Antithrombin III as determined by serial ELISA analysis of specimens obtained at the time of restaging MRIs shown in FIG. 6.

FIG. 8 shows a Western blot analysis of Complement Factor H at the same time points as FIG. 6A-D. The results show increased expression of this protein correlates with progression of the disease. In particular, time point C shows an increase in Complement Factor H after progression of disease and time point D shows reduction of Complement Factor H levels in resolution of disease.

FIG. 9A-D show MRI results for a second patient. Time point A reflects CNS lymphoma before autologuous stem cell transplant (ASCT). MRI and CSF cytology were negative. FIG. 9B shows a later time point where relapse was undetected by cytology or MRI. FIG. 9C shows radiographic remission after whole brain radiation therapy. FIG. 9D shows no significant evidence for lymphoma in spite of new symptoms (new neurologic deterioration, cranial nerve deficits, gait instability), and the patient had negative CSF cytology.

FIG. 10 shows serial measurement of Antithrombin III by ELISA for the CNS lymphoma progression depicted in the MRI of FIG. 9. CNS lymphoma progression is reflected by the rise in CSF concentration of Antithrombin III at time point B. Neurologic deterioration by time point D as well as high levels of Antithrombin III suggested a second relapse but repeat neuroimaging and CSF cytologic examination at this time could not document recurrent tumor.

FIG. 11 shows Western blot Analysis for protein Complement Factor H in CSF for a CNS lymphoma patient at the time points depicted in FIG. 9A-D. At time point B, relapse (three new intraparenchymal brain tumors) was undetected by cytology or MRI but was detected by protein marker. Time point D shows that Complement Factor H was persistently elevated, even though relapse was undetected by MRI or cytology.

FIG. 12 shows that the level of Antithrombin III in CNS lymphoma patients who received intrathecal rituximab declines in CSF in those patients who exhibited clinical response (had clearance of tumor); however, decreases in CSF concentration of Antithrombin III were slower or undetectable in those patients who did not respond to intrathecal rituximab.

FIG. 13A-F show specificity/sensitivity of markers through ROC curves. FIG. 13A shows an ROC curve for fibrinogen beta chain, gi 399492 (AUC=1); FIG. 13B shows an ROC curve for a component identified with an m/z value of 412.53, and a retention time of 40.34 minutes (AUC=1); FIG. 13C shows an ROC curve for secretogranin I, gi 134461 (AUC=0.99); FIG. 13D shows an ROC curve for a component identified with an m/z value of 636.28, and a retention time of 27.57 minutes (AUC=1); FIG. 13E shows an ROC curve for a component identified with an m/z value of 955.48, and a retention time of 54.8 minutes ______ (AUC=0.98); FIG. 13F shows an ROC curve for a component identified with an m/z value of 365.86, and a retention time of 35.06 minutes (AUC=1).

### DETAILED DESCRIPTION OF THE INVENTION

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers. Unless defined otherwise, all scientific and technical terms are to be understood as having the same meaning as commonly used in the art to which they pertain. For the purposes of the present invention, the following terms are defined below.

The invention generally relates to the identification of a large number of polypeptides and related molecules that are differentially expressed in cerebrospinal fluid of patients with CNS lymphoma as compared to patients without CNS lymphoma. As used herein, CNS lymphoma refers to primary CNS lymphoma and dissemination of lymphoma to the CNS. Without being bound by theory, it is believed that the present invention represents the first identification and validation of CSF-based biomarkers for CNS lymphoma.

As used herein, the term “marker” includes polypeptide markers and polynucleotide markers. For clarity of disclosure, aspects of the invention will be described with respect to “polypeptide markers” and “polynucleotide markers.” However, statements made herein with respect to “polypeptide markers” are intended to apply to other polypeptides of the invention. Likewise, statements made herein with respect to “polynucleotide” markers are intended to apply to other polynucleotides of the invention, respectively. Thus, for example, a polynucleotide described as encoding a “polypeptide marker” is intended to include a polynucleotide that encodes: a polypeptide marker, a polypeptide that has substantial sequence identity to a polypeptide marker, modified polypeptide markers, fragments of a polypeptide marker, precursors of a polypeptide marker and successors of a polypeptide marker, and molecules that comprise a polypeptide marker, homologous polypeptide, a modified polypeptide marker or a fragment, precursor or successor of a polypeptide marker (e.g., a fusion protein).

As used herein, the term “polypeptide” refers to a polymer of amino acid residues that has at least 5 contiguous amino acid residues, e.g., 5, 6, 7, 8, 9, 10, 11 or 12 or more amino acids long, including each integer up to the full length of the polypeptide. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component. Also included are antibodies produced by a subject in response to overexpressed polypeptide markers.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from a CNS lymphoma subject (or a subject who is suspected of having CNS lymphoma, or is at risk of developing CNS lymphoma) if the level or activity of the marker is higher or lower, respectively, compared to the level of the marker in a sample (or set of samples) obtained from a non-CNS lymphoma subject, or a reference value or range.

The markers identified as being differentially expressed in CNS lymphoma vs. normal controls (see Examples) are of significant biologic interest. Briefly, CSF samples were obtained from patients with CNS lymphoma and from patients without CNS lymphoma. All samples were separated into a high molecular weight fraction, containing proteins with molecular weights greater than about 5-kDa, and a low molecular weight fraction containing free floating peptides and small molecules having a molecular weight of less than about 5-kDa. After removal of high abundance proteins, the high molecular weight fraction was digested with trypsin. Each fraction was separated by chromatographic means and analyzed by mass spectrometry. The high molecular weight fraction was submitted to proteolysis before analysis by mass spectrometry as discussed in the Example. The resulting spectra were compared to identify individual markers that showed significant association with CNS lymphoma.

In addition to the discovery of biomarkers that can be used individually or in any combination in assays and kits for the diagnosis of, prognosis of, or other evaluation or study of CNS lymphoma, the biomarkers not previously recognized to play a role in the disease process of CNS lymphoma can now be studied in more detail and/or be used as targets for the discovery of other modulators of disease or therapeutic agents.

Tables 1-7 provide polypeptide markers that were found at significantly different levels in CSF samples obtained from patients with CNS lymphoma than in samples from control patients. The Tables show data obtained from clinical studies of CNS lymphoma patients. Two separate clinical studies were undertaken as described in Example 1. In the first study (hereinfter Study 1), nine patients were enrolled. In the second study (hereinafter Study 2), eight patients were enrolled. The analysis of patient samples in both studies was performed as described in Example 2. The results of Study 1, including identified markers, are disclosed in U.S. Provisional Patent Application Ser. No. 60/656,749, which is incorporated by reference herein in its entirety. The results of study 1 are also shown in Table 1A. CNS Lymphoma Study 1: Diseased vs. Control. Table 1A shows a component-level view of the molecules tracked with p<0.01 or CountDiffmin of +/−7 (see the definition of CountDiffmin below). The results of Study 2 are shown in Table 1B: CNS Lymphoma Study 2: Diseased vs. Control. Table 1B shows a component-level view of the molecules tracked with p<0.05 or CountDiffmin of +/−6 (see the definition of CountDiffmin below). Reference to Table 1 herein includes Table 1A and Table 1B. Table 2:CNS Lymphoma Study 2 Summary: Diseased vs. Control, shows a protein summary view of the data in Table 1B. Since a single polypeptide may generate a number of components (fragments), the summary table shows a smaller number of identified molecules than Table 1. Table 3: Comparison of Study 1 and Study 2 for p<0.05 at the Component Level, shows all of statistically significantly changing molecules found in Study 1 and Study 2, at the component-level. Table 4: Common Proteins from Study 1 and Study 2, shows a compilation of all proteins that were significant in either of Study 1 or Study 2, even if only one component was seen in one Study and none showed up in the other. Table 5: Common Proteins from Study 1 and Study 2 with >2 Peptides per Protein in Either Study, lists proteins that were tracked by at least two components in at least one of the two studies, including proteins that were tracked by two components in one study but not tracked at all in the other study. Table 6: Common Proteins from Study 1 and Study 2 with >2 Peptides per Protein in Each Study, reflects polypeptides for which at least two components were tracked in each of Study 1 and Study 2. Table 7: Table 7: Significant Proteins in Study 1 and Study 2 for p<0.04 or Other Evidence of Signifigance, shows an analysis where the data from the two studies were combined as if one study and re-analyzed, pulling out components with p<0.04, and four additional components that were added based on other supporting evidence comparing the two independent studies.

The abbreviations used in the Tables will be familiar to those of skill in the art. For clarity, “Comp. #” refers to the component number; “m/z” refers to the mass-to-charge ratio; “R.T. (min)” refers to the retention time in minutes; “z” refers to the charge; “M+H” refers to the protonated molecular ion mass; “gi #” refers to the GenInfo Identifier; “Exp. Ratio” refers to the expression ratio, which is a ratio of mean group intensities indicating the relative normalized signal for disease group compared to control; “Mods” refers to modifications; “DM(mD)” refers to difference in mass in milliDalton between observed and predicted values; “DM(ppm)” refers to difference in mass in parts per million between observed and predicted values; fold change (an expression change factor where positive indicates a relative intensity increase and negative indicates a relative decrease versus the control); “CountDiff” refers to the count difference between study groups or the difference between two study groups of the number of subjects reporting a detectable intensity for a given component; CountDiffmin refers to the minimum number by which two groups may differ in count, to be categorized as a CountDiff, and therefore to be considered as significantly differentially expressed; and where available, identification number from NCBI's reference sequence database (Accession # and gi #) and additional information (e.g., the name or sequence of the peptide marker as contained in the NCBI queried database and database searching using the Mascot or TurboSEQUEST programs). All information associated with the publicly available identifiers and accession numbers in any of the tables described herein, including the nucleic acid sequences of the associated genes, is incorporated herein by reference in its entirety. Given the name of the protein (also referred to herein as the “full protein”; indicated as “Protein”), other peptide fragments of such measured proteins may be obtained (by whatever means), and such other peptide fragments are included within the scope of the invention. The methods of the present invention may be used to evaluate fragments of the listed molecules as well as molecules that contain an entire listed molecule, or at least a significant portion thereof (e.g., measured unique epitope), and modified versions of the markers. Accordingly, such fragments, larger molecules and modified versions are included within the scope of the invention.

As one of skill in the art will appreciate, the physical and chemical properties presented in the Tables are sufficient to distinguish the component from other materials. In some embodiments, the markers set forth in the Tables 1-7 are each identified on the mass to charge ratio (m/z), chromatographic retention time (RT), the charge state of a molecular ion (z), protonated parent mass (M+H), and expression ratio (exp. ratio). In other embodiments, the components are uniquely identified by the mass to charge ratio (m/z) and the retention time (RT).

Homologs and alleles of the polypeptide markers of the invention can be identified by conventional techniques. As used herein, a homolog to a polypeptide is a polypeptide from a human or other animal that has a high degree of structural similarity to the identified polypeptides. Identification of human and other organism homologs of polypeptide markers identified herein will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., brain) and use the nucleic acids that encode polypeptides identified herein to screen the library for related nucleotide sequences. The screening preferably is performed using high-stringency conditions (described elsewhere herein) to identify those sequences that are closely related by sequence identity. Nucleic acids so identified can be translated into polypeptides and the polypeptides can be tested for activity.

Many of the polypeptides listed in Tables 1-7 are fragments of complete proteins (“parent proteins”), either because they were present as fragments in the sample or as a result of the trypsin digestion that was performed during the processing of certain fractions of the sample (see Example). The parent proteins are included as polypeptide markers. In many cases, the sequence of the parent protein can be ascertained from the amino acid sequence of the fragment by searching a protein sequence database. The tables of the invention include the identification of proteins that include an identified polypeptide marker, although proteins comprising such polypeptides are not limited to those provided in the tables.

Additionally, the present invention includes polypeptides that have substantially similar sequence identity to the polypeptides of the present invention. As used herein, two polypeptides have “substantial sequence identity” when there is at least about 70% sequence identity, at least about 80% sequence identity, at least about 90% sequence identity, at least about 95% sequence identity or at least about 99% sequence identity between their amino acid sequences, or when polynucleotides encoding the polypeptides are capable of forming a stable duplex with each other under stringent hybridization conditions. For example, conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods. For example, upon determining that a peptide is a CNS lymphoma-associated polypeptide, one can make conservative amino acid substitutions to the amino acid sequence of the peptide, and still have the polypeptide retain its specific antibody-binding characteristics. Additionally, one skilled in the art will realize that allelic variants and SNPs will give rise to substantially similar polypeptides and the same or substantially similar polypeptide fragments.

A number of comparison studies were performed to identify the polypeptide markers listed using various groups of CNS lymphoma and non-CNS lymphoma patients. The Tables list markers that were found to be differentially present with statistical significance. Accordingly, it is believed that these biomarkers are indicators of CNS lymphoma. Where a polypeptide marker was found to be statistically significant in a plurality of studies, the data associated with the observations of highest statistical significance is presented. Accordingly, in one aspect, the invention provides polypeptides biomarkers of CNS lymphoma. In one embodiment, the invention provides an isolated component described in Tables 1-7. In another embodiment, the invention provides a polypeptide having substantial sequence identity with a component set forth in Tables 1-7. In another embodiment, the invention provides a molecule that comprises a foregoing polypeptide. As used herein, a compound is referred to as “isolated” when it has been separated from at least one component with which it is naturally associated. For example, a polypeptide can be considered isolated if it is separated from contaminants including metabolites, polynucleotides and other polypeptides. Isolated molecules can be either prepared synthetically or purified from their natural environment. Standard quantification methodologies known in the art can be employed to obtain and isolate the molecules of the invention.

Some variation is inherent in the measurements of the physical and chemical characteristics of the markers. The magnitude of the variation depends to some extent on the reproductively of the separation means and the specificity and sensitivity of the detection means used to make the measurement. Preferably, the method and technique used to measure the markers is sensitive and reproducible.

The retention time and mass to charge ratio may vary to some extent depending on a number of factors relating to the protocol used for the chromatography and the mass spectrometry parameters (e.g., solvent composition, flow rate). Preferably, sample preparation and analysis conditions are carefully controlled. However, one of skill in the art will appreciate that the possibility of contamination or measurement of artifacts can never be completely eliminated.

The data set forth in the Tables reflects the method that was used to detect the markers. When a sample is processed and analyzed as described in the Example, the retention time of the marker is about the value stated for the marker; that is, within about 10% of the value stated, within about 5% of the value stated, or within about 1% of the value stated, and the marker has a mass to charge ratio of about the value stated for the marker; that is, within about 10% of the value stated, within about 5% of the value stated, or within about 1% of the value stated. Accordingly, in another embodiment, the invention provides a polypeptide having (i) a mass-to-charge value and (ii) an RT value of about the values stated, respectively, for a component described in Tables 1-7. In another embodiment, the invention provides a molecule that comprises a foregoing polypeptide.

Polypeptide identifications in Tables 1-7 reflect a single polypeptide appearing in a database for which the component was a match. In general, the polypeptide is the largest polypeptide found in the database. Such a selection is not meant to limit the polypeptide to those disclosed in Tables 1-7, however. Accordingly, in another embodiment, the invention provides a polypeptide that is a fragment, precursor, successor or modified version of a marker described in Tables 1-7. For example the following polypeptides appear in Table 1: antithrombin-III precursor, complement factor H isoform a precursor [Homo sapiens], and complement factor H precursor (H factor 1), fibulin 1 precursor, splice form C—human fibulin-1 precursor. Such precursors are typically larger then the processed form. The invention therefore includes the successor molecules (i.e., processed proteins) Antithrombin III, Complement Factor H, and Fibulins. In another embodiment, the invention includes a molecule that comprises a foregoing fragment, precursor, successor or modified polypeptide.

Markers that are particularly useful may be identified by validation experiments. As used herein, validation refers to establishing further evidence that an identified marker is a marker. For example, Example 3 describes the validation of Antithrombin III, Complement Factor H, and EGF-containing fibulin-like extracellular matrix protein 1 (EFEMP1), also known as Fibulin-3 (FBLN3).

In other embodiments, the marker is a member of the Fibulin family. The Fibulins are a family of secreted glycoproteins. Fibulins are characterized by repeated epidermal-growth-factor-like domains and a unique C-terminal structure (FBLC motif) that forms a globular domain. Currently, six distinct Fibulin genes, encoding at least nine protein products generated by alternative splicing, have been identified. These proteins are known by a number of names in the art, as Fibulin-1, Fibulin-2, Fibulin-3, Fibulin-4, Fibulin-5, and Fibulin-6. Alternative splice variants are known for at least Fibulins 1-4. For example variants for Fibulin 1 include Fibulin 1A, Fibulin 1B, Fibulin 1C, and Fibulin 1D. As indicated in the attached tables, at least two members of the fibulin family, EFEMP1 (Fibulin 3) and Fibulin 1 are upregulated in CNS lymphoma patients. Without being bound by theory, it is believed that additional members of the fibulin family are markers of CNS cancers, including CNS lymphoma. Considerable evidence is available pointing towards a structural role for fibulins within the extracellular matrix. Fibulins have been shown to modulate cell morphology, growth, adhesion and motility. The dysregulation of certain fibulins occurs in a range of human disorders, including cancer. Gallagher, et al. Trends Mol. Med. 2005 11:336-40.

Certain embodiments of the present invention utilize a plurality of biomarkers that have been identified herein as being differentially expressed in subjects with CNS lymphoma. As used herein, the terms “patient,” “subject” and “a subject who has CNS lymphoma” and “CNS lymphoma subject” are intended to refer to subjects who have been diagnosed with CNS lymphoma. The terms “non-subject” and “a subject who does not have CNS lymphoma” are intended to refer to a subject who has not been diagnosed with CNS lymphoma. A non-CNS lymphoma subject may be healthy and have no other disease, or they may have a disease other than CNS lymphoma.

The plurality of biomarkers within the above-limitation includes at least two or more biomarkers (e.g., at least 2, 3, 4, 5, 6, and so on, in whole integer increments, up to all of the possible biomarkers) identified by the present invention, and includes any combination of such biomarkers. Such biomarkers are selected from any of the polypeptides listed in the tables provided herein, and polynucletide encoding any of the polypeptides listed in the Tables.

The polypeptide and polynucleotide markers of the invention are useful in methods for diagnosing CNS lymphoma, determining the extent and/or severity of the disease, monitoring progression of the disease and/or response to therapy. Such methods can be performed in human and non-human subjects. The markers are also useful in methods for treating CNS lymphoma and for evaluating the efficacy of treatment for the disease. Such methods can be performed in human and non-human subjects. The markers may also be used as pharmaceutical compositions or in kits. The markers may also be used to screen candidate compounds that modulate their expression. The markers may also be used to screen candidate drugs for treatment of CNS lymphoma. Such screening methods can be performed in human and non-human subjects.

Polypeptide markers may be isolated by any suitable method known in the art. Native polypeptide markers can be purified from natural sources by standard methods known in the art (e.g., chromatography, centrifugation, differential solubility, immunoassay). In one embodiment, polypeptide markers may be isolated from a CSF sample using the chromatographic methods disclosed herein. In another embodiment, polypeptide markers may be isolated from a sample by contacting the sample with substrate-bound antibodies or aptamers that specifically bind to the marker.

The present invention also included polynucleotide markers related to the polypeptide markers of the present invention. In one aspect, the invention provides polynucleotides that encode the polypeptides of the invention. The polynucleotide may be genomic DNA, cDNA, or mRNA transcripts that encode the polypeptides of the invention. In one embodiment, the invention provides polynucleotides that encode a polypeptide described in Tables 1-7, or a molecule that comprises such a polypeptide.

In another embodiment, the invention provides polynucleotides that encode a polypeptide having substantial sequence identity with a component set forth in Tables 1-7, or a molecule that comprises such a polypeptide.

In another embodiment, the invention provides polynucleotides that encode a polypeptide having (i) a mass-to-charge value and (ii) an RT value of about the values stated, respectively, for a marker described in Tables 1-7, or a molecule that comprises such a polypeptide.

In another embodiment, the invention provides polynucleotides that encode a polypeptide having (i) a mass-to-charge value within 10% (more particularly within 5%, more particularly within 1%) and (ii) an RT value within 10% (more particularly within 5%, more particularly within 1%) of the m/z and RT values stated, respectively, for a component described in Tables 1-7, or a molecule that comprises such polypeptide.

In another embodiment, the invention provides polynucleotides that encode a polypeptide that is a fragment, precursor, successor or modified version of a marker described in Tables 1-7, or a molecule that comprises such polypeptide.

In another embodiment, the invention provides polynucleotides that have substantial sequence similarity to a polynucleotide that encodes a polypeptide that is a fragment, precursor, successor or modified version of a marker described in Tables 1-7, or a molecule that comprises such polypeptide. Two polynucleotides have “substantial sequence identity” when there is at least about 70% sequence identity, at least about 80% sequence identity, at least about 90% sequence identity, at least about 95% sequence identity or at least about 99% sequence identity between their amino acid sequences or when the polynucleotides are capable of forming a stable duplex with each other under stringent hybrization conditions. Such conditions are described elsewhere herein. As described above with respect to polypeptides, the invention includes polynucleotides that are allelic variants, the result of SNPs, or that in alternative codons to those present in the native materials as inherent in the degeneracy of the genetic code.

In some embodiments, the polynucleotides described may be used as surrogate markers of CNS lymphoma. Thus, for example, if the level of a polypeptide marker is increased in CNS lymphoma-patients, an increase in the mRNA that encodes the polypeptide marker may be interrogated rather than the polypeptide marker (e.g., to diagnose CNS lymphoma in a subject).

Polynucleotides encoding the polypeptides markers listed in Tables 1-7 can be used to screen existing genomic, cDNA or expression libraries to find the gene that encodes the polynucleotide of the invention. A library is typically screened using a probe that is complementary either to the polynucleotide that encodes a polypeptide in Tables 1-7, or to its complement, under conditions which promote hybridization, including stringent hybridization. Hybridization is monitored by any suitable method known in the art. Once located, the gene can be cloned. The protein product of a gene that encodes a fragment of a polynucleotide marker is also included as a polypeptide marker. Alternatively, the sequence of the polynucleotide that encode a polypeptide listed in Tables 1-7 can be used to search databases such as SWISS-PROT and GenBank, which will provide the gene sequence(s) comprising the nucleic acid sequence, and the amino acid sequence of the gene product.

Polynucleotide markers may be isolated by any suitable method known in the art. Native polynucleotide markers may be purified from natural sources by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, polynucleotide markers may be synthesized by any suitable chemical or recombinant method known in the art. In one embodiment, for example, the makers can be synthesized using the methods and techniques of organic chemistry. In another embodiment, a polynucleotide marker can be produced by polymerase chain reaction (PCR).

The invention also provides markers that have been identified as differentially expressed in patients with a CNS cancer, including CSF lymphoma and metastatic brain cancers, including Antithrombin III, Complement Factor H, and EFEMP1 (EGF-containing fibulin-like extracellular matrix protein 1 (EFEMP1), also known as Fibulin-3 (FBLN3)). For example, Complement Factor H is a marker generally associated with CNS cancers, as described in Example 3B, and as shown in FIG. 5. Complement Factor H is associated with primary CNS lymphoma, Non-Hodgkins lymphoma metastatic to the brain, and carcinoma metastatic to the brain (lung and breast primary tumors).

The present invention also encompasses molecules which specifically bind the polypeptide or polynucleotide markers of the present invention. In one aspect, the invention provides molecules that specifically bind to a polypeptide marker or a polynucleotide marker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA, immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity. As used herein, an aptamer is a non-naturally occurring nucleic acid having a desirable action on a target a desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. in the preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

In one aspect, the invention provides antibodies or apatmers that specifically bind to a component described in Tables 1-7, or to a molecule that comprises a foregoing component (e.g., a protein comprising a polypeptide identified in a table of the invention).

In another embodiment, the invention provides antibodies or aptamers that specifically bind to a polypeptide having substantial sequence identity with a component set forth in Tables 1-7, or to a molecule that comprises a foregoing polypeptide.

In another embodiment, the invention provides antibodies or aptamers that specifically bind to a component having (i) a mass-to-charge value and (ii) an RT value of about the values stated, respectively, for a marker described in Tables 1-7, or to a molecule that comprises a foregoing component.

In another embodiment, the invention provides antibodies or aptamers that specifically bind to a component having (i) a mass-to-charge value within 10% (more particularly within 5%, more particularly within 1%) and (ii) an RT value within 10% (more particularly within 5%, more particularly within 1%) of the m/z and RT values stated, respectively, for a component described in Tables 1-7, or to a molecule that comprises a foregoing component.

In another embodiment, the invention provides antibodies or aptamers that specifically bind to a component that is a fragment, modification, precursor or successor of a marker described in Tables 1-7, or to a molecule that comprises a foregoing component.

In another embodiment, the invention provides antibodies or aptamers that specifically bind to a polypeptide marker or a polynucleotide marker that is structurally different from a component specifically identified in Tables 1-7 but has the same (or nearly the same) function or properties, or to a molecule that comprises a foregoing component.

Another embodiment of the present invention relates to a plurality of antibodies, or antigen binding fragments thereof, or aptamers for the detection of the expression of biomarkers differentially expressed in patients with CNS lymphoma. The plurality of antibodies, or antigen binding fragments thereof, or aptamers consists of antibodies, or antigen binding fragments thereof, or aptamers that selectively bind to proteins differentially expressed in patients with CNS lymphoma, and that can be detected as protein products using antibodies or aptamers. In addition, the plurality of antibodies, or antigen binding fragments thereof, or aptamers comprises antibodies, or antigen binding fragments thereof, or aptamers that selectively bind to proteins or portions thereof (peptides) encoded by any of the genes from the tables provided herein.

According to the present invention, a plurality of antibodies, or antigen binding fragments thereof, or aptamers refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, or aptamers including antibodies representing all of the biomarkers described herein, or antigen binding fragments thereof.

Certain antibodies that specifically bind polypeptide markers polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

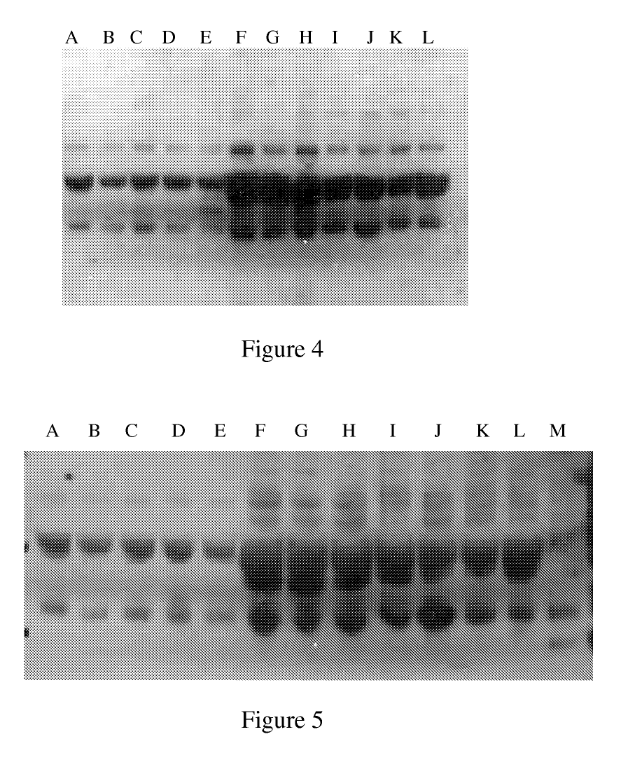

Antibodies or aptamers that specifically bind markers of the invention can be used, for example, in methods for detecting components described in Tables 1-7 using methods and techniques well-known in the art. In some embodiments, for example, the antibodies are conjugated to a detection molecule or moiety (e.g., a dye, and enzyme) and can be used in ELISA or sandwich assays to detect markers of the invention. FIG. 4 shows the detection of Complement Factor H in a patient with CNS lymphoma by a western blot analysis, for example.

In another embodiment, antibodies or aptamers against a polypeptide marker or polynucleotide marker of the invention can be used to assay a tissue sample (e.g., a thin cortical slice or biopsy sample) for the marker. The antibodies or aptamers can specifically bind to the marker, if any, present in the tissue sections and allow the localization of the marker in the tissue. Similarly, antibodies or aptamers labeled with a radioisotope may be used for in vivo imaging or treatment applications.

Another aspect of the invention provides compositions comprising a polypeptide or polynucleotide marker of the invention, a binding molecule that is specific for a polypeptide or polynucleotide marker (e.g., an antibody or an aptamer), an inhibitor of a polypeptide or polynucleotide marker, or other molecule that can increase or decrease the level or activity of a polypeptide marker or polynucleotide marker. Such compositions may be pharmaceutical compositions formulated for use as a therapeutic.

In one embodiment, the invention provides a composition that comprises a polypeptide or polynucleotide marker of the invention, such as a component described in Tables 1-7, a polypeptide having substantial sequence identity with a component or having (i) a mass-to-charge value and (ii) an RT value of about the values, respectively, for a component, or a molecule comprising such a component.

Alternatively, the invention provides a composition that comprises a component that is a fragment, modification, precursor or successor of a marker described in Tables 1-7, or to a molecule that comprises a foregoing component.

In another embodiment, the invention provides a composition that comprises a polynucleotide that binds to a polypeptide or a molecule that comprises a foregoing polynucleotide.

In another embodiment, the invention provides a composition that comprises an antibody or aptamer that specifically binds to a polypeptide or a molecule that comprises a foregoing antibody or aptamer.

In another embodiment, the invention provides a composition that comprises a modulator of the level or activity of a polypeptide marker (e.g., an inhibitor of a polypeptide marker, an antisense polynucleotide which is complementary to a polynucleotide that encodes a polypeptide marker), or a molecule that comprises a foregoing modulator.

Such compositions may be pharmaceutical compositions. Typically, a pharmaceutical composition comprises a therapeutically effective amount of an active agent and is formulated with a suitable excipient or carrier. The invention also provides pharmaceutical compositions for the treatment of CNS lymphoma. These compositions may include a marker protein and/or nucleic acid of the invention (e.g., for those markers which are decreased in quantity or activity in CNS lymphoma samples versus non-CNS lymphoma samples), and can be formulated as described herein. Alternately, these compositions may include an antibody or aptamer which specifically binds to a marker protein of the invention and/or an antisense polynucleotide which is complementary to a polynucleotide marker of the invention (e.g., for those markers which are increased in quantity or activity in CNS lymphoma samples versus non-CNS lymphoma samples), and can be formulated as described herein.

The pharmaceutical compositions of the invention can be prepared in any suitable manner known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

The present invention also provides methods of detecting the biomarkers of the present invention. The practice of the present invention employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a polypeptide or polynucleotide is included in the invention. It is within the ability of one of ordinary skill in the art to determine which method would be most appropriate for measuring a specific marker. Thus, for example, a ELISA assay may be best suited for use in a physician's office while a measurement requiring more sophisticated instrumentation may be best suited for use in a clinical laboratory. Regardless of the method selected, it is important that the measurements be reproducible.

The markers of the invention can be measured by mass spectrometry, which allows direct measurements of analytes with high sensitivity and reproducibility. A number of mass spectrometric methods are available. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Mass spectrometers that allow time-of-flight (TOF) measurements have high accuracy and resolution and are able to measure low abundant species, even in complex matrices like serum or CSF.

For protein markers, quantification can also be based on derivatization in combination with isotopic labeling, such as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another. Quantification can also be based on the isotope dilution method by spiking in an isotopically labeled peptide or protein analogous to those being measured. Furthermore, quantification can also be determined without isotopic standards using the direct intensity of the analyte comparing with another measurement of a standard in a similar matrix.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In one embodiment, the markers are measured using mass spectrometry in connection with a separation technology, such as liquid chromatography-mass spectrometry. In particular, coupling reverse-phase liquid chromatography to high resolution, high mass accuracy ESI time-of-flight (TOF) mass spectroscopy allows spectral intensity measurement of a large number of biomolecules from a relatively small amount of any complex biological material. Analyzing a sample in this manner allows the marker (characterized by a specific RT and m/z) to be determined and quantified.

As will be appreciated by one of skill in the art, many other separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (RT). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. “Deconvoluting” software is available to separate overlapping peaks. The retention time depends to some degree on the conditions employed in performing the liquid chromatography separation. The preferred conditions, those used to obtain the retention times that appear in the Tables, are set forth in the Examples. The mass spectrometer preferably provides high mass accuracy and high mass resolution. The mass accuracy of a well-calibrated Micromass TOF instrument, for example, is reported to be approximately 2 mDa, with resolution m/m exceeding 5000.

In other preferred embodiments, the level of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker. Any molecule that is capable of specifically binding to a marker is included within the invention. In some embodiments, the binding molecules are antibodies or antibody fragments. In other embodiments, the binding molecules are non-antibody species, such as aptamers. Thus, for example, the binding molecule may be an enzyme for which the marker is a substrate. The binding molecules may recognize any epitope of the targeted markers.

As described above, the binding molecules may be identified and produced by any method accepted in the art. Methods for identifying and producing antibodies and antibody fragments specific for an analyte are well known. Methods for identifying and producing aptamers to a target are also well-known. Examples of other methods used to identify the binding molecules include binding assays with random peptide libraries (e.g., phage display) and design methods based on an analysis of the structure of the marker.

The markers of the invention also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labeled probe (e.g., an antibody or aptamer) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5× SSC, followed by at least one wash at about 68° C. in about 0.1×-0.5× SSC). Other hybridization conditions, and for example, those most useful with nucleic acid arrays, will be known to those of skill in the art.

The present invention also includes methods of diagnosing CNS lymphoma and related methods. In general, it is expected that the biomarkers described herein will be measured in combination with other signs, symptoms and clinical tests of CNS lymphoma, such as MRI or CSF abnormalities, or CNS lymphoma biomarkers reported in the literature. Likewise, more than one of the biomarkers of the present invention may be measured in combination. Measurement of the biomarkers of the invention along with any other markers known in the art, including those not specifically listed herein, falls within the scope of the present invention. Markers appropriate for this embodiment include those that have been identified as increased or decreased in samples obtained from CNS lymphoma samples compared with samples from non-CNS lymphoma samples (e.g., markers described in Tables 1-7), as well as antibodies produced by a patient in response to an increased level of a polypeptide marker. Other markers appropriate for this embodiment include fragments, precursors, successors and modified versions of such markers, polypeptides having substantial sequence identity to such markers, components having an m/z value and RT value of about the values set forth for the markers described in Tables 1-7, and molecules comprise one of the foregoing. Other appropriate markers for this embodiment will be apparent to one of skill in the art in light of the disclosure herein.

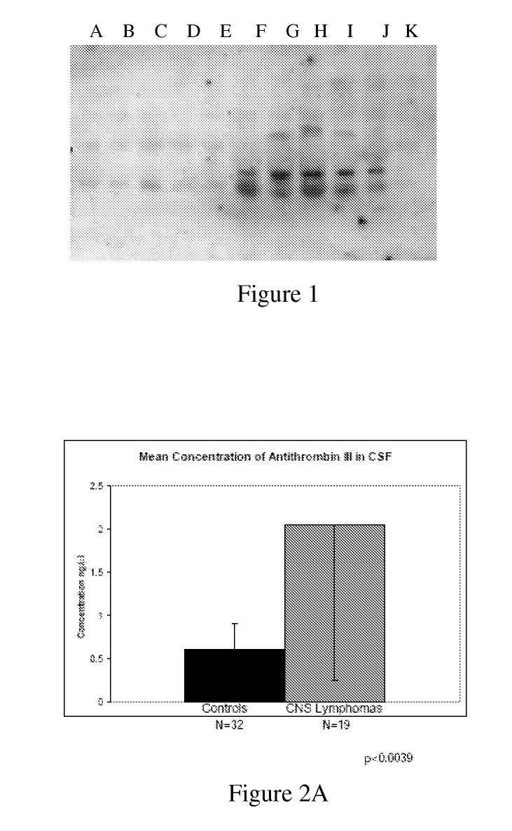

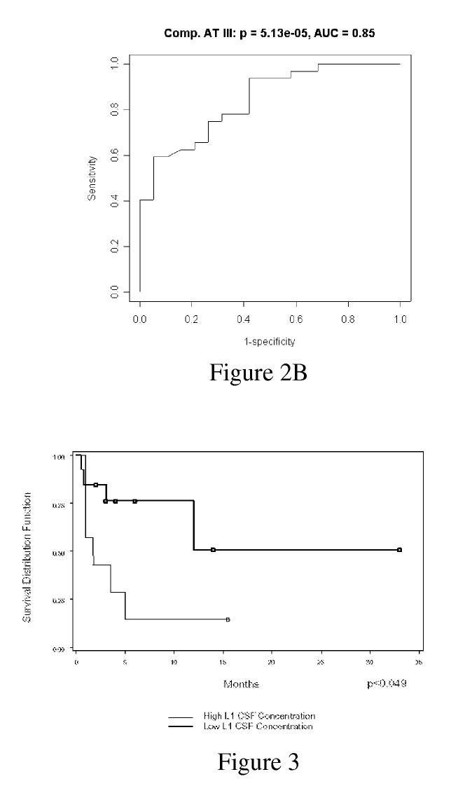

In one embodiment, the present invention provides a method for determining whether a subject has CNS lymphoma. In another aspect, the invention provides methods for diagnosing CNS lymphoma in a subject. These methods comprise obtaining a biological sample from a subject suspected of having CNS lymphoma, or at risk for developing CNS lymphoma, detecting the level or activity of one or more biomarkers in the sample, and comparing the result to the level or activity of the marker(s) in a sample obtained from a non-CNS lymphoma subject, or to a reference range or value. As used herein, the term “biological sample” includes a sample from any body fluid or tissue (e.g., serum, plasma, blood, cerebrospinal fluid, urine). Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as having CNS lymphoma or as not having CNS lymphoma. In some embodiments, CNS lymphoma is diagnosed in the patient if the expression level of the biomarker or biomarkers in the patient sample is statistically more similar to the expression level of the biomarker or biomarkers that has been associated with CNS lymphoma than the expression level of the biomarker or biomarkers that has been associated with the normal controls. As an example, the markers Antithrombin III, Complement Factor H, and EFEMP1 (EGF-containing fibulin-like extracellular matrix protein 1 (EFEMP1), also known as Fibulin-3 (FBLN3) can be used to diagnose CNS Lymphoma. FIG. 1, FIG. 4, and FIG. 5 show these markers (as detected by a Western blot) are elevated in CNS lymphoma, as compared to benign conditions. Additionally, the concentration of Antithrombin III as determined by ELISA is significantly higher in the CSF of patients with CNS lymphoma compared to the CSF of control subjects (FIG. 2).

What is presently referred to as CNS lymphoma may turn out to be a number of related, but distinguishable conditions. Classifications may be made, and these types may be further distinguished into subtypes. Any and all of the various forms of CNS lymphoma are intended to be within the scope of the present invention. Indeed, by providing a method for subsetting patients based on biomarker measurement level, the compositions and methods of the present invention may be used to uncover and define various forms of the disease.

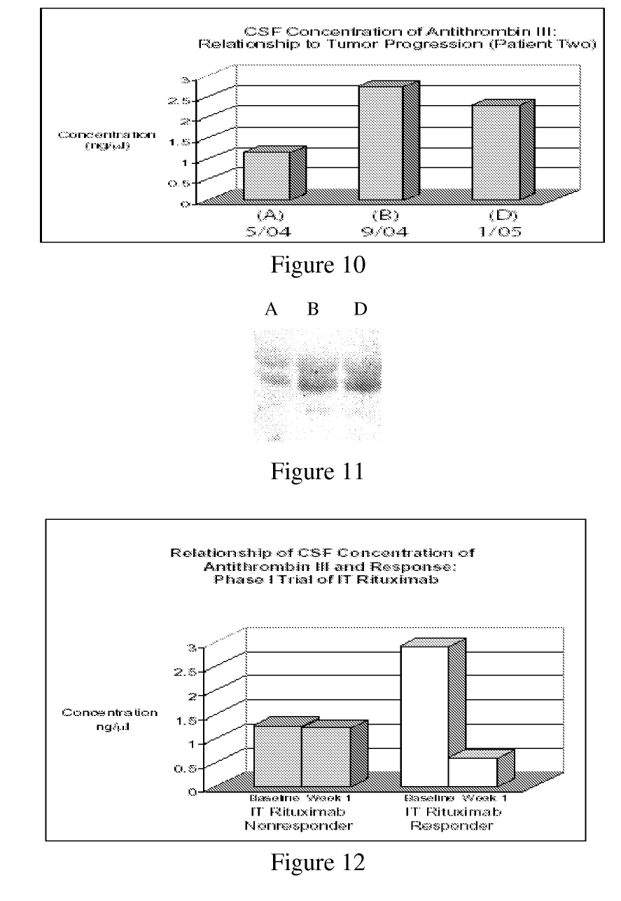

The methods of the present invention may be used to make the diagnosis of CNS lymphoma, independently from other information such as the patient's symptoms or the results of other clinical or paraclinical tests. As an example, the presence of Antithrombin III may be used to diagnose CNS lymphoma even in the absence of corroborating MRI or cytology data (see Example 3C; FIG. 9B, FIG. 10B, and FIG. 11B). However, the methods of the present invention may be used in conjunction with such other data points (see Example 3C; FIG. 9A, FIG. 10A, and FIG. 11A).

Because a diagnosis is rarely based exclusively on the results of a single test, the method may be used to determine whether a subject is more likely than not to have CNS lymphoma, or is more likely to have CNS lymphoma than to have another disease, based on the difference between the measured and standard level or reference range of the biomarker. Thus, for example, a patient with a putative diagnosis of CNS lymphoma may be diagnosed as being “more likely” or “less likely” to have CNS lymphoma in light of the information provided by a method of the present invention. As an example, the presence of Antithrombin III may be used to indicate that a patient is more likely to have CNS lymphoma than not in the absence of corroborating MRI or cytology data (see Example 3C; FIG. 9D, FIG. 10D, and FIG. 11D). If a plurality of biomarkers are measured, at least one and up to all of the measured biomarkers must differ, in the appropriate direction, for the subject to be diagnosed as having (or being more likely to have) CNS lymphoma. For example, Antithrombin III and Complement Factor H will be increased in CNS lymphoma. In some embodiments, such difference is statistically significant.

The biological sample may be of any tissue or fluid, including a CSF or tissue sample, but other biological fluids or tissue may be used. Possible biological fluids include, but are not limited to, serum, plasma, and urine. In some embodiments, the level of a marker may be compared to the level of another marker or some other component in a different tissue, fluid or biological “compartment.” Thus, a differential comparison may be made of a marker in tissue and CSF. It is also within the scope of the invention to compare the level of a marker with the level of another marker or some other component within the same compartment.

As will be apparent to those of ordinary skill in the art, the above description is not limited to making an initial diagnosis of CNS lymphoma, but also is applicable to confirming a provisional diagnosis of CNS lymphoma or “ruling out” such a diagnosis. Furthermore, an increased or decreased level or activity of the marker(s) in a sample obtained from a subject suspected of having CNS lymphoma, or at risk for developing CNS lymphoma, is indicative that the subject has or is at risk for developing CNS lymphoma.

The invention also provides a method for determining a subject's risk of developing CNS lymphoma, the method comprising obtaining a biological sample from a subject, detecting the level or activity of a marker in the sample, and comparing the result to the level or activity of the marker in a sample obtained from a non-CNS lymphoma subject, or to a reference range or value wherein an increase or decrease of the marker is correlated with the risk of developing CNS lymphoma.

The invention also provides methods for determining the stage or severity of CNS lymphoma, the method comprising obtaining a biological sample from a subject, detecting the level or activity of a marker in the sample, and comparing the result to the level or activity of the marker in a sample obtained from a non-CNS lymphoma subject, or to a reference range or value wherein an increase or decrease of the marker is correlated with the stage or severity of the disease. For example, the marker Antithrombin III is correlated with overall survival as described in Example 3A and as shown in FIG. 2. Patients with a high Antithrombin III level had a lower overall survival rate than patients with a low Antithrombin III level. Additionally, FIG. 7 shows that Antithrombin III (as detected by a Western blot) is present in a patient with CNS Lymphoma (A), was still present after chemotherapy (B) and progression of the disease (C), but was not present after a high-dose treatment after which the subject was believed to be cancer free (D). Furthermore, FIG. 11 shows the presence of Complement Factor H (as detected by a Western blot) in a CNS lymphoma patient (A), after a relapse (B), and after the appearance of additional symptoms (D).

In another aspect, the invention provides methods for monitoring the progression of the disease in a subject who has CNS lymphoma, the method comprising obtaining a first biological sample from a subject, detecting the level or activity of a marker in the sample, and comparing the result to the level or activity of the marker in a second sample obtained from the subject at a later time, or to a reference range or value wherein an increase or decrease of the marker is correlated with progression of the disease.

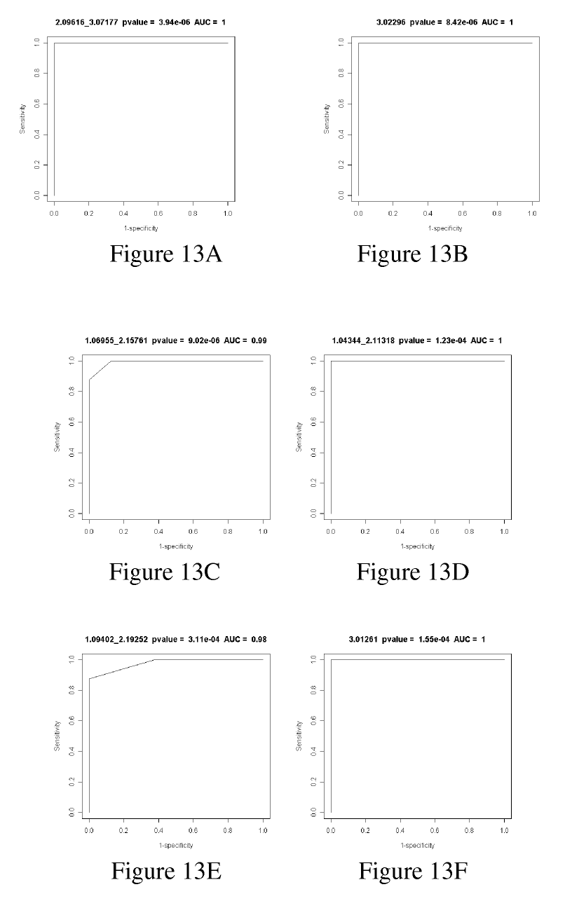

As indicated in Tables 1-7, some of the marker measurement values are higher in CNS lymphoma samples, while others are lower. A significant difference in the appropriate direction in the measured value of one or more of the markers indicates that the patient has (or is more likely to have, or is at risk of having, or is at risk of developing, and so forth) CNS lymphoma. If only one biomarker is measured, then that value must increase or decrease to indicate CNS lymphoma. If more than one biomarker is measured, then a diagnosis of CNS lymphoma can be indicated by a change in only one biomarker, all biomarkers, or any number in between. In some embodiments, multiple markers are measured, and a diagnosis of CNS lymphoma is indicated by changes in multiple markers. For example, a panel of markers may include markers that are increased in level or activity in CNS lymphoma subject samples as compared to non-CNS lymphoma subject samples, markers that are decreased in level or activity in CNS lymphoma subject samples as compared to non-CNS lymphoma subject samples, or a combination thereof. Measurements can be of (i) a biomarker of the present invention, (ii) a biomarker of the present invention and another factor known to be associated with CNS lymphoma (e.g., MRI scan or CSF cytology); (iii) a plurality of biomarkers of the present invention, (iv) a plurality of biomarkers comprising at least one biomarker of the present invention and at least one biomarker reported in the literature (e.g., monoclonality of light chain expression in B-lymphocytes) or (v) any combination of the foregoing. Furthermore, the amount of change in a biomarker level may be an indication of the relatively likelihood of the presence of the disease. A number of markers identified in the present invention have excellent specificity and sensitivity. Sensitivity means the probability that a test result will be positive when the disease is present (true positive rate, expressed as a percentage). Specificity means the probability that a test result will be negative when the disease is not present (true negative rate, expressed as a percentage). The ability of a test to discriminate diseased cases from normal cases is evaluated using Receiver Operating Characteristic (ROC) curve analysis. FIG. 13A-F show ROC curves for six markers of the invention. The area under the curve for these markers ranges from 0.98-1 with 1 being ideal and 0.5 being random.

The marker may be detected in any biological sample obtained from the subject, by any suitable method known in the art (e.g., immunoassays, hybridization assay) see supra.

In an alternative embodiment of the invention, a method is provided for monitoring a CNS lymphoma patient over time to determine whether the disease is progressing. The specific techniques used in implementing this embodiment are similar to those used in the embodiments described above. The method is performed by obtaining a biological sample, such as CSF or serum, from the subject at a certain time (t₁); measuring the level of at least one of the biomarkers in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁-t₀). A further deviation of a marker in the direction indicating CNS lymphoma, or the measurement of additional increased or decreased CNS lymphoma markers, would suggest a progression of the disease during the interval. Subsequent sample acquisitions and measurements can be performed as many times as desired over a range of times t₂ t₀. Such serial monitoring is described in Example 3C.

The ability to monitor a patient by making serial marker level determinations would represent a valuable clinical tool. Rather than the limited “snapshot” provided by a single test, such monitoring would reveal trends in marker levels over time. In addition to indicating a progression of the disease, tracking the marker levels in a patient could be used to predict exacerbations or indicate the clinical course of the disease. For example, the relative concentration of Antithrombin III and Complement Factor H provides information which suggests the presence of minimal residual disease undetectable by MRI or by CSF cytology (See Example 3C, FIG. 9B, FIG. 10B, and FIG. 11B).

Additionally, as will be apparent to one of skill in the art, the biomarkers of the present invention could be further investigated to distinguish between any or all of the known forms of CNS lymphoma or any later described types or subtypes of the disease. In addition, the sensitivity and specificity of any method of the present invention could be further investigated with respect to distinguishing CNS lymphoma from other diseases or to predict relapse or remission.

In an analogous manner, administration routes of a particular drug can be examined. The drug can be administered differently to different subject populations, and measurements corresponding to each administration route analyzed to determined if the differences in the inventive biomarkers before and after drug administration are significant. Results from the different routes can also be compared with each other directly.

In another aspect, the invention provides methods for screening candidate compounds for use as therapeutic compounds. In one embodiment, the method comprises screening candidate compounds for those that bind to a polypeptide or polynucleotide molecule of the invention. Candidate compounds that bind to markers can be identified using any suitable method or technique known in the art.

In one embodiment, a candidate compound or a control is contacted with marker and the ability of the candidate compound to form stable complexes is determined (e.g., flow cytometry, immunoprecipitation). The candidate compound, the marker, an aptamer or an antibody that specifically binds either may be labeled to facilitate detection. The candidate molecule or marker may be immobilized on a solid support (e.g., a bead).

In another embodiment, cells expressing a polypeptide marker are contacted with a candidate compound or a control and the ability of the candidate compound to form stable complexes with the cells is determined. The candidate compound or the marker may be labeled to facilitate detection.

In an analogous manner, the markers of the present invention can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period as appropriate for the intervention. The measurements before and after could then be compared to determine whether or not the treatment had an effect effective. For example, measurement of the change in CSF concentration of Antithrombin III as a surrogate marker for early response to treatment correlates with the therapeutic efficacy of a given treatment modality, e.g., Rituxamib (See Example 4). As will be appreciated by one of skill in the art, the determination may be confounded by other superimposed processes (e.g., an exacerbation of the disease during the same period).

In a further additional embodiment, the markers may be used to screen candidate drugs, for example, in a clinical trial, to determine whether a candidate drug is effective in treating CNS lymphoma. At time to, a biological sample is obtained from each subject in population of subjects diagnosed with CNS lymphoma. Next, assays are performed on each subject's sample to measure levels of a biological marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ t₀. In such a study, a different sub-population of the subject population serves as a control group, to which a placebo is administered. The same procedure is then followed for the control group: obtaining the biological sample, processing the sample, and measuring the biological markers to obtain a measurement chart.

Specific doses and delivery routes can also be examined. The method is performed by administering the candidate drug at specified dose or delivery routes to subjects with CNS lymphoma; obtaining biological samples, such as CSF or serum, from the subjects; measuring the level of at least one of the biomarkers in each of the biological samples; and, comparing the measured level for each sample with other samples and/or a standard level. Typically, the standard level is obtained by measuring the same marker or markers in the subject before drug administration. Depending upon the difference between the measured and standard levels, the drug can be considered to have an effect on CNS lymphoma. If multiple biomarkers are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating CNS lymphoma.

In a typical embodiment, a subject population having CNS lymphoma is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed. The stratification of the patient population may be made based on the levels of biomarkers of the present invention.

In general, a number of statistical considerations must be made in designing the trial to ensure that statistically significant changes in biomarker measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time to, a biological sample is obtained from each subject in the population. Next, an assay or variety of assays are performed on each subject's sample to measure levels of particular biomarkers of the invention. The assays can use conventional methods and reagents, as described above. If the sample is blood, then the assays typically are performed on either serum or plasma. For other fluids or tissues, additional sample preparation steps are included as necessary before the assays are performed. The assays measure values of at least one of the biological markers described herein. In some embodiments, only a single marker is monitored, while in other embodiments, a combination of factors, up to the total number of markers, is monitored. The markers may also be monitored in conjunction with other measurements and factors associated with CNS lymphoma (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner as the sample acquired from the subject population before drug administration (the “tₒ sample”). The same assays are performed on the t₁ sample as on the to sample to obtain measurement values. Subsequent sample acquisitions and measurements can be performed as many times as desired over a range of times t₂ tₙ.

Typically, a different sub-population of the subject population is used as a control group, to which a placebo is administered. The same procedure is then followed for the control group: obtaining the biological sample, processing the sample, and measuring the biological markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various biomarkers are now available for each subject. The different measurement values are compared and analyzed to determine whether the biological markers changed in the expected direction for the drug group but not for the placebo group, indicating that the candidate drug is effective in treating the disease. In preferred embodiments, such change is statistically significant. The measurement values at time t₁ for the group that received the candidate drug are compared with standard measurement values, preferably the measured values before the drug was given to the group, i.e., at time tₒ. Typically, the comparison takes the form of statistical analysis of the measured values of the entire population before and after administration of the drug or placebo. Any conventional statistical method can be used to determine whether the changes in biological marker values are statistically significant. For example, paired comparisons can be made for each biomarker using either a parametric paired t-test or a non-parametric sign or sign rank test, depending upon the distribution of the data.

In addition, tests may be performed to ensure that statistically significant changes found in the drug group are not also found in the placebo group. Without such tests, it cannot be determined whether the observed changes occur in all patients and are therefore not a result of candidate drug administration.

As indicated in Tables 1-7, some of the marker measurement values are higher in samples from CNS lymphoma patients, while others are lower. The nonadjusted p-values shown were obtained by univariate analysis. A significant change in the appropriate direction in the measured value of one or more of the markers indicates that the drug is effective. If only one biomarker is measured, then that value must increase or decrease to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Measurements can be of both biomarkers of the present invention and other measurements and factors associated with CNS lymphoma (e.g., measurement of biomarkers reported in the literature and/or MRI imaging). Furthermore, the amount of change in a biomarker level may be an indication of the relatively efficacy of the drug.

In addition to determining whether a particular drug is effective in treating CNS lymphoma, biomarkers of the invention can also be used to examine dose effects of a candidate drug. There are a number of different ways that varying doses can be examined. For example, different doses of a drug can be administered to different subject populations, and measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose. Based on the results of drug screenings, the markers of the invention may be used as theragnostics; that is, they can be used to individualize medical treatment.

In another aspect, the invention provides a kit for detecting a polypeptide or polynucleotide marker.

In another aspect, the invention provides a kit for diagnosing CNS lymphoma in a patient including reagents for detecting at least one polypeptide or polynucleotide marker in a biological sample from the subject.

In another aspect, the invention provides a kit for screening candidate compounds including reagents for detecting stable complexes between the candidate compound and a polynucleotide or polynucleotide marker.

The kits of the invention may comprise one or more of the following: an antibody, wherein the antibody specifically binds with a polypeptide marker, an aptamer, wherein the aptamer specifically binds with a polypeptide marker, a labeled binding partner to the antibody or aptamer, a solid phase upon which is immobilized the antibody, aptamer or its binding partner, a polynucleotide probe that can hybridize to a polynucleotide marker, pairs of primers that under appropriate reaction conditions can prime amplification of at least a portion of a polynucleotide marker or a polynucleotide encoding a polypeptide marker (e.g., by PCR), instructions on how to use the kit, and a label or insert indicating regulatory approval for diagnostic or therapeutic use.

The invention further includes polynucleotide or polypeptide microarrays comprising polypeptides of the invention, polynucleotides of the invention, or molecules, such as antibodies or aptamers, which specifically bind to the polypeptides or polynucleotides of the present invention. In this aspect of the invention, standard techniques of microarray technology are utilized to assess expression of the polypeptides biomarkers and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Polynucleotide arrays, particularly arrays that bind polypeptides of the invention, also can be used for diagnostic applications, such as for identifying subjects that have a condition characterized by expression of polypeptide biomarkers, e.g., CNS lymphoma.

The invention also provides methods for treating CNS lymphoma, as well as other diseases or conditions, by providing a therapeutic agent to a subject that increases or decreases the level or activity of at least one marker of the invention.

In one embodiment, the method comprises administering a therapeutic agent to a subject that increases level or activity of at least one polypeptide or polynucleotide marker of the invention that is decreased in samples obtained from CNS lymphoma subjects compared to samples obtained from non-CNS lymphoma subjects or to a reference range or value. In one embodiment, the therapeutic agent is Retuximab.

In another embodiment, the method comprises administering a therapeutic agent to a subject that decreases the level of at least one polypeptide or polynucleotide marker of the invention that is increased in samples obtained from CNS lymphoma subjects compared to samples obtained from non-CNS lymphoma subjects or to a reference range or value.

In another embodiment, the method further comprises first obtaining a sample from an CNS lymphoma subject, determining the presence, level or activity of at least one marker of the invention in the sample compared to samples obtained from a non-CNS lymphoma subject or to a reference range or value. If the marker is increased in the sample obtained from the CNS lymphoma subject, a therapeutic agent that decreases the level of the marker is administered to the patient. If the marker is decreased in the sample obtained from the CNS lymphoma subject, a therapeutic agent that increases the level of the marker is administered to the subject.

Therapeutic agents include but are not limited to polypeptide markers, polynucleotide markers, molecules comprising a polypeptide marker or polynucleotide marker, antibodies to polypeptide marker or polynucleotide marker, aptamers having polypeptide marker targets, modulators of the level or activity a polypeptide or polynucleotide marker (e.g., an inhibitor, anti-sense polynucleotides) or compositions comprising one or more of the foregoing.

Generally, the therapeutic agents used in the invention are administered to the subject in an effective amount. An “effective amount” is typically the amount that is sufficient to obtain beneficial or desired clinical results. The effective amount is generally determined by a physician with respect to a specific patient and is within the skill of one in the art. Factors that may be taken into account in determining an effective amount include those relating to the condition being treated (e.g., type, stage, severity) as well as those relating to the subject (e.g., age, weight).

The level or activity of a polypeptide marker may be increased or decreased by any suitable technique or method known in the art. The level of a polypeptide marker may be increased by providing the polypeptide marker to a subject. Alternatively, the level of a polypeptide marker may be increased by providing a polynucleotide that encodes the polypeptide marker (e.g., gene therapy). For those polypeptide markers with enzymatic activity, compounds or molecules known to increase that activity may be provided to the subject.

The level of a polypeptide marker may be decreased by providing antibodies or aptamers specific for the polypeptide marker to the subject. Alternatively, the level of a polypeptide marker may be decreased by providing a polynucleotide that is “anti-sense” to the polynucleotide that encodes the polypeptide marker, or that encodes dysfunctional proteins. For those polypeptide markers with enzymatic activity, compounds or molecules known to decrease that activity (e.g., inhibitor or antagonist).

The therapeutic compounds described herein may be administered alone or in combination with another therapeutic compound, or other form of treatment. The compounds may be administered to the subjects in any suitable manner known in the art (e.g., orally, topically, subcutaneously, intradermally, intramuscularly, intravenously, intra-arterially, intrathecally). Metabolites may be combined with an excipient and formulated as tablets or capsules for oral administration. Polypeptides may be formulated for parenteral administeration to avoid denaturation by stomach acids. For polynucleotides, vectors may be constructed for administration to the subject by a virus or other carrier. In a typical embodiment, cDNA is delivered to target cells (e.g., bone marrow cells) that are later reintroduced into the subject for expression of the encoded protein. A therapeutic composition can be administered in a variety of unit dosage forms depending upon the method of administration.

### EXAMPLES

### Example 1

### Sample Collection

Subjects were enrolled in a clinical study in which the inclusion criteria included subjects that: (a) are immunocompetent (non-AIDS); (b) are greater than eighteen years old; (c) are free of active infection; and (d) have atraumatic or minimally traumatic CSF. Exclusion criteria included: (a) overt Leptomeningeal infection (Bacterial Meningitis); (b) AIDS; and (c) traumatic specimen (bloody). Nine patients were enrolled in Study 1, and eight patients were enrolled in Study 2.

CSF samples were collected from the same number of subjects in a Control group. The Control group included normal individuals being screened for CNS malignancy or subjects undergoing treatment for benign pituitary lesions. In a first study, CSF samples were collected from nine subjects in a Lymphoma group. In a second study, CSF samples were collected from eight subjects in a Lymphoma group. The Lymphoma group included individuals with active non-Hodgkin's lymphoma (usually B cell) in the leptomeninges or brain parenchyma.

CSF from subjects were obtained from lumbar sac or Ommaya reservoir and processed within 1 hour of collection, immediately subjected to centrifugation by clinical centrifuge to pellet cells and supernatant immediately frozen at <−70 degrees C.

### Example 2

### Marker Identification

Markers of the invention were identified using the CSF samples described immediately above.

A. CSF Proteome. A high molecular weight fraction (“CSF proteome”) was separated from the CSF samples using a 5 kDa molecular weight cut-off spin filter (Millipore Corp., Bedford, Mass.). The CSF proteome was diluted with PBS buffer, about pH 6. To increase the effective dynamic range of the measurements, the most abundant proteins, albumin, IgG, antitrypsin, IgA, transferrin and haptoglobin, were substantially depleted using an antibody-based protein removal column (Agilent, Palo Alto, Calif.). The remaining proteins were denatured using guanidine hydrochloride, disulfide bonds were reduced using dithiotreitol, and sulfhydryl groups were carboxymethylated using iodoacetic acid/NaOH. The denaturant and reduction-alkylation reagents were removed by buffer exchange. After digestion of the proteins using modified Trypsin (Promega Corp., Madison, Wis.), the mixture was lyophilized to a powder, dissolved in formic acid, desalted, dried again Strong-cation exchange (SCX) chromatography. The samples, each re-dissolved, were injected onto a Spherisorb SCX column, with 5-micron diameter particles. A KCl-salt gradient elutes the analytes over time. The eluent is collected on a fraction collector and for this example, three fractions were collected. Each of these fractions were then dried, re-dissolved in 0.1% formic acid, desalted with a C18 solid-phase extraction (SPE) cartridge (Sep/Pak cartridge by Waters Corp., Milford, Mass.), dried, and re-dissolved in 0.1% formic acid before injection for LC-MS Analysis.

The tryptic peptides were profiled by liquid chromatography-electrospray ionization-mass spectrometry (LC-ESI-MS) on a high-resolution time-of-flight (TOF) instrument. For LC separation, an online 0.3 mm diameter×15 cm long column was packed with C18 reverse-phase (RP) material (Micro-Tech Scientific, Inc., Vista, Calif.). Peptides retained on the RP column were eluted with increasing concentration of acetonitrile (ACN). The eluate from the column flowed into the ESI-TOF MS (Micromass LCT™, Waters Corp., Milford, Mass.). Individual molecules were tracked across samples and their differential expression determined.

B. Tandem MS. LC/MS/MS was used for separation and identification of differentially expressed components. Using remaining samples from the study or similar samples, the eluate from the LC configuration (described above) flowed into a LTQ ion trap mass spectrometer (ThermoFinnigan, Waltham, Mass.) or microQ-TOF mass spectrometer (Waters Corp. Milford, Mass.).

C. Peptide Identification Data Acquisition. MS/MS spectra obtained from the LTQ (Thermo Electron Corp., San Jose, Calif.) and Q-TOF (Waters Corp.) mass spectrometers were used to identify peptides. A more accurate parent ion molecular weight is obtained from a parallel analysis using the LCT orthogonal-injection ESI-TOF (Micromass). Accuracy of the LCT detection is as good as ˜10 ppm using the natural internal calibration of known peptides. In comparison, accuracy of the LTQ ion trap is ±0.5 Da (˜1000 ppm). This data is then examined by a database searching approach (described below). In addition, de novo amino acid sequence analysis programs can be used to obtain at least partial sequence analysis. Increased resolution (˜5,000) and accuracy of the LCT TOF instrument significantly limits the range of possible peptides that are candidates, thus allowing focused database searches; this is a valuable contribution for making correct identifications especially in the case of low signal-to-noise mass peaks.

D. Peptide and Protein Identification Data Analysis. TurboSEQUEST software (Thermo Electron Corp, San Jose, Calif.), or similar software such as Mascot (Matrix Science LTD, London, UK), is used to identify peptides and proteins. Link A J, et al. (1999) “Direct analysis of protein complexes using mass spectrometry.” Nature Biotechnol 17:676-82. TurboSEQUEST uses protein or DNA databases, both public and private. In the case of enzymatically digested proteins, an in silico digestion of the associated proteins produces peptides with amino acid sequences theoretically revealed by a computational cleavage according to known rules; these are used to compare against the raw data. Looking up a particular molecular weight with a given mass uncertainty gives a selection of possible peptides (and hence proteins) that can give rise to those peaks. The in silico digestion can include a small number of PTMs. However, database approaches such as TurboSEQUEST will not work to identify peptides or proteins that are not already in the database. In that case, de novo peptide sequencing software and BLAST searching can be used.

E. Post-Translational Modification (PTM). A number of methods were used to detect PTM of the identified polypeptides. Using the known fixed mass that a PTM adds, TurboSEQUEST or Mascot software can identify at least three PTMs on a peptide in a single search.

F. Differential Quantification. Proteins and peptides were quantified relative to the same, corresponding molecules in a different sample, usually a control or normal sample. This differential expression approach relies on the assumption that biological samples consist of complex mixtures of multiple biological components, of which only some are relevant to the comparison. The majority of components are relatively constant for the same individual over time or across subject populations. The majority of components whose concentrations do not vary across samples are used as an intrinsic internal standard to normalize the concentrations of components that do vary. The method also relies on the inherent reproducibility of ionization for ESI. The high reproducibility of ESI is measured by the coefficient of variation. The majority of peaks have a CV less then 20%, aside from biological variance. The validity of this approach is discussed in more detail in Wang et al. (2003) and U.S. Pat. No. 6,835,927.

G. Determination of p-Value. Univariate hypothesis tests for each mass spectrometry component were used for the comparisons of means between control and prostate cancer groups. Parametric or non-parametric tests were used, depending on the normality of the data. If the data were approximately normally distributed, the parametric statistic was used (t-test); if not, the nonparametric statistic (Wilcoxon test) was used. Goodness-of-fit statistics (Shapiro-Wilk) and tests of skewness and kurtosis were performed to assess the normality of each biometric component. The results of these tests are presented in form of a p-value per component. The p-value represents the probability of a false positive on a univariate level.

H. Results. Results of studies performed on samples from CNS lymphoma subjects and from control subjects without CNS lymphoma are presented in Tables 1-7.

### Example 3

### Validation of Markers

The differential expression of three CNS lymphoma biomarkers identified by LC/MS using immunoassays were validated with available reagents. Results of Western blots for two of these CNS lymphoma biomarkers, Antithrombin III and Complement factor H are shown, as well as quantitative ELISA data for Antithrombin III.

A. Antithrombin III. Western blot analysis was used to validate the high relative expression of Antithrombin III in the CSF of patients with brain lymphoma (FIG. 1). As shown in FIG. 1, a high relative concentration of Antithrombin III was detected in the CSF of six out of seven patients with CNS lymphoma compared to patients with benign neurologic conditions. A monoclonal antibody against human Antithrombin III was used. CSF was normalized for total protein, 10 μg, then subjected to SDS/PAGE, proteins transferred to nitrocellulose membrane and probed with mouse monoclonal anti human Antithrombin III antibody. Extending this analysis further, an ELISA for detection of Antithrombin III in the CSF was generated, using commercially-available antibodies which specifically recognize this protein. ELISA results confirm that levels of Antithrombin III are significantly higher in the CSF of patients with CNS lymphoma (n=19) compared to control subjects (n=32). Mean Antithrombin III concentration as determined by ELISA in control subjects is 0.6 ng/ml; mean Antithrombin III concentration in CNS lymphoma patients is 2.0 ng/ml. p<0.0039. (FIG. 2A).

FIG. 2B shows a high level of diagnostic accuracy of CSF concentration of Antithrombin III in the discrimination between CNS lymphoma and non-neoplastic controls through an ROC curve with an AUC of 0.85.

CSF from CNS lymphoma patients were obtained from 19 immunocompetent patients with primary or secondary CNS lymphomas, 18 of which were B-cell lymphomas and 1 of which was a T-cell lymphoma. The control subject population (n=32) for this analysis was different from the set of controls previously analyzed by LC/MS and consisted of patients with meningeal infection, neurosarcoid, multiple sclerosis, early dementia, normal pressure hydrocephalus, benign pituitary neoplasms, peripheral neuropathy, cerebrovascular disease and patients with systemic acute leukemias and lymphomas who underwent lumbar punctures as part of routine staging procedures which ruled out CNS involvement.

One of the reasons efforts were focused on the validation of the differential expression of Antithrombin III in the CSF in CNS lymphoma patients is microarray data which not only demonstrated for the first time its expression in B-cell CNS lymphomas, but which also provided evidence that high expression of this gene is associated with short survival (less than six months). Biopsy and resection specimens of CNS lymphoma tumors used for this microarray analysis were obtained from an independent set of immunocompetent CNS lymphoma patients who were diagnosed and treated at M.D. Anderson Cancer Center, Harvard University, and The University of California at San Francisco. None of the CSF which were used for LC/MS analysis or for validation experiments (Western blot or ELISA) were from biopsy or surgical specimens which were used for this microarray analysis. DNA microarrays were constructed using a 20,000 clone set (Research Genetics). Mean antithrombin III gene expression was 2.2-fold higher in patients with short survival (less than six months) than in patients with longer survival (p<0.003). In light of this gene expression data, the outcome of the 19 CNS lymphoma patients whose CSF was subjected to ELISA for determination of concentration of Antithrombin III was analyzed. Using a concentration of 2.0 ng/ml (the mean CSF Antithrombin III concentration in CNS lymphoma) as a cut-off, the survival of those patients with high CSF protein levels of Antithrombin III (above the mean) were compared with those whose CSF Antithrombin III concentration were below the mean. A trend toward high CSF concentration of Antithrombin III and shorter overall survival (p<0.049) was found. (FIG. 3). Moreover, the majority of patients with high CSF concentrations of Antithrombin III (greater than 2.0 ng/ml) succumbed to tumor progression; the majority of patients with Antithrombin III concentrations less than 2.0 ng/ml were still alive at the time of this retrospective analysis (p<0.03). This data suggests for the first time that measurement of Antithrombin III in the CSF may not only facilitate diagnosis of CNS lymphoma but may also provide prognostic information, either alone or in combination with other markers.

B. Complement Factor H. The high relative expression of Complement Factor H in the CSF of patients with CNS lymphoma was validated using immunoblot analysis with a monoclonal antibody raised against human Complement Factor H. Patients with CNS lymphomas had higher expression of Complement Factor H in CSF than patients with multiple sclerosis, neurosarcoid or other benign conditions (FIG. 5).

As shown in FIG. 5, Complement Factor H protein from CSF migrates as two distinct bands on SDS/PAGE (arrows at approximately 60 KD and 52 KD). The immunoblot demonstrates high relative Complement Factor H expression in CSF of CNS lymphoma patients as well as evidence for post-translational processing of the higher molecular weight peptide. Recombinant human Complement Factor H peptides (far right) serve as a control. CSF is normalized for total protein, 10 μg, before CSF proteins are subjected to SDS/PAGE, transferred to membrane and probed with mouse monoclonal anti-human Complement Factor H antibody.

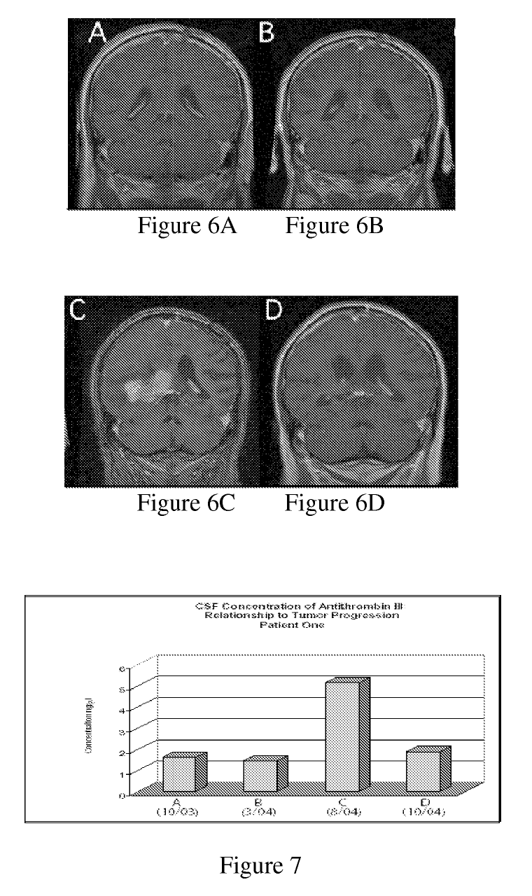

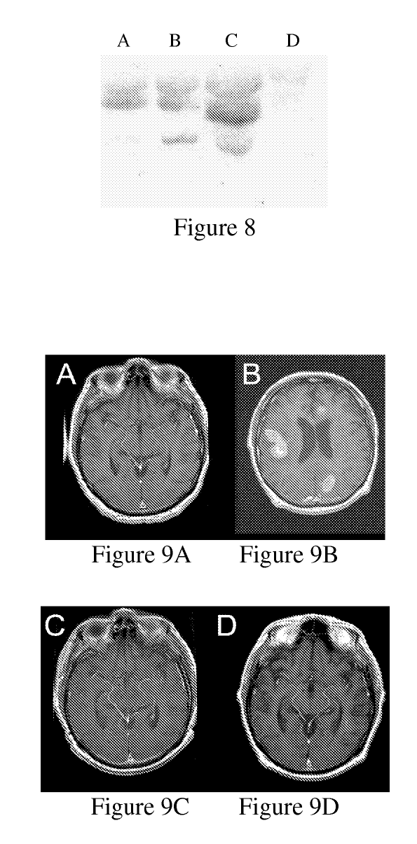

C. Serial Analysis of CSF expression of Antithrombin III and Complement Factor H. Serial analysis of the CSF expression of Antithrombin III and Complement Factor H was performed in CSF specimens in individual CNS lymphoma patients (n=8). These studies provide evidence that the relative CSF concentration of Antithrombin III as well as Complement Factor H reflect the course of disease. This has been demonstrated both by ELISA (for Antithrombin III) as well as by immunoblot for Antithrombin III and for Complement Factor H. Two CNS lymphoma case studies are presented which document not only the relationship between CSF expression of these putative biomarkers with disease status but also the utility of these molecules to provide clinical data regarding the presence of minimal residual disease undetectable by CSF cytology or by neuroimaging. FIG. 6-FIG. 8 show serial MRI, measurement of Antithrombin III by ELISA, and relative concentration of Complement Factor H in one patient, respectively. CNS lymphoma progression and therapeutic response are reflected by the rise and fall in CSF concentrations of Antithrombin III and Complement Factor H at various times. Time point A correlates to the presence of CNS lymphoma; B is a later time point showing recurrent leptomeningeal CNS lymphoma. Time point C shows progression of the disease, while time point D shows resolution of the disease after high-dose systemic methotrexate chemotherapy.

FIG. 9-FIG. 11 show serial MRI, measurement of Antithrombin III by ELISA, and relative concentration of Complement Factor H in a second patient, respectively. CNS lymphoma progression and therapeutic response are reflected by the rise and fall in CSF concentrations of Antithrombin III and Complement Factor H at various times. Time point A correlates to the presence of CNS lymphoma prior to autologous stem cell transplant, while B is relapse after the transplant and C is remission after whole brain radiation. Neurologic deterioration at time point D suggested a second relapse but repeat neuroimaging and CSF cytologic examination at this time could not document recurrent tumor. Measurement of Antithrombin III and Complement Factor H were elevated at this time, suggesting residual malignancy. The patient succumbed to progressive disease shortly thereafter.

D. EFEMP1 (EGF-containing fibulin-like extracellular matrix protein 1 (EFEMP1), also known as Fibulin-3 (FBLN3)). The validation of EFEMP1 as a marker was performed in a manner similar to that of Antithrombin III and Complement Factor H, as shown in FIG. 4.

### Example 4

### Antithrombin III and Clinical Response to Rituximab

The relationship between early changes in the CSF concentration of Antithrombin III and clinical response to intrathecal rituximab has been analyzed. Rituximab is the first monoclonal antibody to receive FDA approval in the treatment of cancer and is indicated in the treatment of large B-cell lymphoma, the most common histology in CNS lymphomas. The intrathecal use of this antibody in treating patients with recurrent CNS lymphomas has been investigated in a phase I multicenter study. Measurement of Antithrombin III CSF concentrations in CNS lymphoma patients who received intrathecal rituximab reproducibly demonstrate that declines in CSF concentration of Antithrombin III occur rapidly, within one week of initiation of intrathecal rituximab in those patients who exhibited clinical response (had clearance of tumor). Declines in CSF concentration of Antithrombin III were slower or undetectable in those patients who did not respond to intrathecal rituximab. (FIG. 12). These results suggest that monitoring the rate of change or absolute change of Antithrombin III concentration in the CSF could be a clinically useful early surrogate biomarker to determine whether a particular therapy is effective in treating a brain tumor such as CNS lymphoma or whether other modalities such as irradiation or other chemotherapies are required, before neurologic deterioration occurs.

### Example 5

### Markers for Other CNS Cancers

CSF expression of certain markers, while quite specific in distinguishing CNS lymphoma from benign neurologic conditions, are also useful as markers of metastatic tumors to the brain. For example, high relative levels of Complement Factor H, EFEMP-1 and Antithrombin III have been detected in each of 5 patients with brain metastatic carcinomas derived from lung and breast primary tumors. Additionally, the CSF biomarkers disclsosed herein may be useful for the early diagnosis of leptomeningeal metastases (carcinomatous meningitis).

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

It should be understood that the foregoing disclosure emphasizes certain specific embodiments of the invention and that all modifications or alternatives equivalent thereto are within the spirit and scope of the invention as set forth in the appended claims.

LENGTHY TABLE REFERENCED HERE
US20070264643A1-20071115-T00001
Please refer to the end of the specification for access instructions.

LENGTHY TABLE REFERENCED HERE
US20070264643A1-20071115-T00002
Please refer to the end of the specification for access instructions.

LENGTHY TABLE REFERENCED HERE
US20070264643A1-20071115-T00004
Please refer to the end of the specification for access instructions.

LENGTHY TABLE REFERENCED HERE
US20070264643A1-20071115-T00005
Please refer to the end of the specification for access instructions.

LENGTHY TABLE REFERENCED HERE
US20070264643A1-20071115-T00006
Please refer to the end of the specification for access instructions.

LENGTHY TABLE REFERENCED HERE
US20070264643A1-20071115-T00007
Please refer to the end of the specification for access instructions.

LENGTHY TABLE REFERENCED HERE
US20070264643A1-20071115-T00008
Please refer to the end of the specification for access instructions.

LENGTHY TABLE REFERENCED HERE
US20070264643A1-20071115-T00009
Please refer to the end of the specification for access instructions.

LENGTHY TABLE REFERENCED HERE
US20070264643A1-20071115-T00010
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070264643A1). An electronic copy of the table will also be available from the USPTO upon request and payment of the fee set forth in 37 CFR 1.19(b)(3).

1. A method for diagnosing CNS lymphoma in a subject, comprising: determining the level of a marker in a biological sample obtained from a subject; comparing the level of the marker in the sample to a reference value, wherein the marker is selected from the group consisting of a polypeptide comprising a marker identified in Tables 1-7, and a polynucleotide encoding a polypeptide comprising a marker identified in Tables 1-7.

2. The method of claim 1, wherein the marker is selected from the group consisting of a polypeptide comprising a marker identified in Tables 2-5, and 7, and a polynucleotide encoding a polypeptide comprising a marker identified in Tables 2-5, and 7.

3-6. (canceled)

7. The method of claim 1, wherein the marker is selected from the group consisting of a polypeptide identified in Tables 1-7.

8. The method of claim 7, wherein the marker is selected from the group consisting of a polypeptide identified in Tables 2-5, and 7.

9-12. (canceled)

13. The method of claim 1, wherein the marker is selected from the group consisting of Antithrombin III, Complement Factor H, and EFEMP1.

14. The method of claim 1, wherein the biological sample is a body fluid.

15. The method of claim 14, wherein the body fluid is selected from the group consisting of blood, serum, plasma, cerebrospinal fluid, urine, and saliva.

16. The method of claim 1, wherein the biological sample is cerebrospinal fluid.

17. The method of claim 1, wherein the marker comprises a polypeptide or fragment thereof.

18. The method of claim 1, wherein the reference value is the level of the marker in at least one sample from a non-CNS lymphoma subject.

19. The method of claim 1, wherein the polypeptide is the marker.

20. The method of claim 1, wherein the polypeptide shares at least about 70% sequence identity with the marker.

21. The method of claim 1, wherein the polypeptide is a modified form of the marker.

22. The method of claim 1, wherein the method further comprises detecting the presence of the polypeptide using a reagent that specifically binds to the polypeptide or a fragment thereof.

23. The method of claim 22, wherein the reagent is selected from the group consisting of an antibody, an antibody derivative, and an antibody fragment.

24. The method of claim 23, wherein the reagent selected from the group consisting of an anti-Antithrombin III antibody, and anti-Complement Factor H antibody, and an anti-Fibulin antibody.

25. The method of claim 1, wherein said method comprises a plurality of markers.

26. The method of claim 25, wherein at least two of the markers are selected from the group consisting of a polypeptide comprising a marker identified in Tables 1-7, and a polynucleotide encoding a polypeptide comprising a marker identified in Tables 1-7.

27-53. (canceled)

54. A method for monitoring CNS lymphoma in a subject, the method comprising: measuring the level of a marker in first biological sample from a subject, wherein the marker is selected from the group consisting of a polypeptide comprising a marker identified in Tables 1-7, and a polynucleotide encoding a polypeptide comprising a marker identified in Tables 1-7; measuring the level of the marker in a second biological sample from a subject, wherein the marker is selected from the group consisting of a polypeptide comprising a marker identified in Tables 1-7, and a polynucleotide encoding a polypeptide comprising a marker identified in Tables 1-7; and comparing the level of the marker measured in the first sample with the level of the marker measured in the second sample, whereby CNS lymphoma in a subject is monitored.

55-58. (canceled)

59. The method of claim 54, wherein the marker is selected from the group consisting of a polypeptide comprising a marker identified in Tables 2-6, and a polynucleotide encoding a polypeptide comprising a marker identified in Tables 2-6.

60-63. (canceled)

64. The method of claim 54, wherein the marker is selected from the group consisting of a polypeptide identified in Tables 1-7.

65. The method of claim 64, wherein the marker is selected from the group consisting of a polypeptide identified in Tables 2-6.

66-69. (canceled)

70. The method of claim 54, wherein the marker is selected from the group consisting of Antithrombin III, Complement Factor H, and EFEMP1.

71. A method of assessing the efficacy of a treatment for CNS lymphoma in a subject, the method comprising comparing: the level of a marker measured in a first sample obtained from the subject before the treatment has been administered to the subject, wherein the marker is selected from the group consisting of a polypeptide comprising a marker identified in Tables 1-7, and a polynucleotide encoding a polypeptide comprising a marker identified in Tables 1-7, and the level of the marker in a second sample obtained from the subject after the treatment has been administered to the subject, wherein a change in the level of the marker in the second sample relative to the first sample is an indication that the treatment is efficacious for treating CNS lymphoma in the subject.

72-73. (canceled)

74. The method of claim 71, wherein the marker is selected from the group consisting of a polypeptide comprising a marker identified in Tables 2-6, and a polynucleotide encoding a polypeptide comprising a marker identified in Tables 2-6.

75. (canceled)

79. The method of claim 71, wherein the marker is selected from the group consisting of a polypeptide identified in Tables 1-7.

80. The method of claim 79, wherein the marker is selected from the group consisting of a polypeptide identified in Tables 2-6.

81-84. (canceled)

85. The method of claim 71, wherein the marker is selected from the group consisting of Antithrombin III, Complement Factor H, and EFEMP1.

86. A method for determining the risk of developing CNS lymphoma in a subject, the method comprising: obtaining a biological sample from the subject; determining the level of a marker in the sample, wherein the marker is selected from the group consisting of a polypeptide comprising a marker identified in Tables 1-7, and a polynucleotide encoding a polypeptide comprising a marker identified in Tables 1-7; comparing the level of the marker in the sample to a reference value; and determining from the results of the comparison that the subject has an increased or decreased risk of developing CNS lymphoma.

87. (canceled)

88. A method for diagnosing CNS cancer in a subject, the method comprising: determining the level of a plurality of markers from one or more biological samples from a subject, wherein at least one of the plurality of markers is selected from the group consisting of a polypeptide comprising a marker identified in Tables 1-7, and a polynucleotide encoding the polypeptides identified in Tables 1-7; and comparing the level of at least one of the plurality of markers to a reference value.

89. The method of claim 88, wherein the CNS cancer is selected from the group consisting of carcinomatous meningitis, and brain metastatic cancers.

90. The method of claim 88, wherein the marker is selected from the group consisting of Antithrombin III, Complement Factor H, and EFEMP1.


SEQUENCE LISTING (1131 sequences)

SEQ ID NO: 1 - protein, 19 aa, Homo sapiens
IIGYTPDLDPETVDDAFAR

SEQ ID NO: 2 - protein, 18 aa, Homo sapiens
VAPEEHPVLLTEAPLNPK

SEQ ID NO: 3 - protein, 9 aa, Homo sapiens
LPNNVLQEK

SEQ ID NO: 4 - protein, 12 aa, Homo sapiens
SGLSTGWTQLSK

SEQ ID NO: 5 - protein, 16 aa, Homo sapiens
NGVAQEPVHLDSPAIK

SEQ ID NO: 6 - protein, 9 aa, Homo sapiens
QSLQEETVR

SEQ ID NO: 7 - protein, 15 aa, Homo sapiens
YVGGQEHFAHLLILR

SEQ ID NO: 8 - protein, 9 aa, Homo sapiens
WFYIASAFR

SEQ ID NO: 9 - protein, 12 aa, Homo sapiens
TYMLAFDVNDEK

SEQ ID NO: 10 - protein, 14 aa, Homo sapiens
EQLGEFYEALDCLR

SEQ ID NO: 11 - protein, 8 aa, Homo sapiens
TEDTIFLR

SEQ ID NO: 12 - protein, 7 aa, Homo sapiens
WFYIASA

SEQ ID NO: 13 - protein, 10 aa, Homo sapiens
EHVAHLLFLR

SEQ ID NO: 14 - protein, 8 aa, Homo sapiens
EQLSLLDR

SEQ ID NO: 15 - protein, 15 aa, Homo sapiens
MEEVEAMLLPETLKR

SEQ ID NO: 16 - protein, 14 aa, Homo sapiens
MEEVEAMLLPETLK

SEQ ID NO: 17 - protein, 19 aa, Homo sapiens
AVLDVFEEGTEASAATAVK

SEQ ID NO: 18 - protein, 18 aa, Homo sapiens
HPNSPLDEENLTQENQDR

SEQ ID NO: 19 - protein, 18 aa, Homo sapiens
LYGSEAFATDFQDSAAAK

SEQ ID NO: 20 - protein, 19 aa, Homo sapiens
RLYGSEAFATDFQDSAAAK

SEQ ID NO: 21 - protein, 12 aa, Homo sapiens
DEELSCTVVELK

SEQ ID NO: 22 - protein, 17 aa, Homo sapiens
DLDSQTMMVLVNYIFFK

SEQ ID NO: 23 - protein, 10 aa, Homo sapiens
ADLSGITGAR

SEQ ID NO: 24 - protein, 15 aa, Homo sapiens
IIVPTDTQNIFFMSK

SEQ ID NO: 25 - protein, 10 aa, Homo sapiens
NLAVSQVVHK

SEQ ID NO: 26 - protein, 16 aa, Homo sapiens
NSPLDEENLTQENQDR

SEQ ID NO: 27 - protein, 14 aa, Homo sapiens
CEGPIPDVTFELLR

SEQ ID NO: 28 - protein, 22 aa, Homo sapiens
TPGAAANLELIFVGPQHAGNYR

SEQ ID NO: 29 - protein, 26 aa, Homo sapiens
LETPDFQLFKNGVAQEPVHLDSPAIK

SEQ ID NO: 30 - protein, 13 aa, Homo sapiens
LGNQEPGGQTALK

SEQ ID NO: 31 - protein, 19 aa, Homo sapiens
LQQVLHAGSGPCLPHLLSR

SEQ ID NO: 32 - protein, 10 aa, Homo sapiens
APHGPGLIYR

SEQ ID NO: 33 - protein, 7 aa, Homo sapiens
CNLLAEK

SEQ ID NO: 34 - protein, 12 aa, Homo sapiens
LGSPSGEVSHPR

SEQ ID NO: 35 - protein, 20 aa, Homo sapiens
DLTGFPGPLNDQDDEDCINR

SEQ ID NO: 36 - protein, 27 aa, Homo sapiens
YNILPEKEEFPFALGVQTLPQTCDEPK

SEQ ID NO: 37 - protein, 15 aa, Homo sapiens
AAQVTIQSSGTFSSK

SEQ ID NO: 38 - protein, 18 aa, Homo sapiens
YSDASDCHGEDSQAFCEK

SEQ ID NO: 39 - protein, 10 aa, Homo sapiens
QGIPFFGQVR

SEQ ID NO: 40 - protein, 8 aa, Homo sapiens
SDIAPVAR

SEQ ID NO: 41 - protein, 17 aa, Homo sapiens
MCPQLQQYEMHGPEGLR

SEQ ID NO: 42 - protein, 8 aa, Homo sapiens
FQVDNNNR

SEQ ID NO: 43 - protein, 19 aa, Homo sapiens
HNVYINGITYTPVSSTNEK

SEQ ID NO: 44 - protein, 9 aa, Homo sapiens
FEVQVTVPK

SEQ ID NO: 45 - protein, 12 aa, Homo sapiens
DMYSFLEDMGLK

SEQ ID NO: 46 - protein, 14 aa, Homo sapiens
IAQWQSFQLEGGLK

SEQ ID NO: 47 - protein, 19 aa, Homo sapiens
LHTEAQIQEEGTVVELTGR

SEQ ID NO: 48 - protein, 18 aa, Homo sapiens
LLIYAVLPTGDVIGDSAK

SEQ ID NO: 49 - protein, 11 aa, Homo sapiens
VGFYESDVMGR

SEQ ID NO: 50 - protein, 21 aa, Homo sapiens
VYDYYETDEFAIAEYNAPCSK

SEQ ID NO: 51 - protein, 10 aa, Homo sapiens
GHFSISIPVK

SEQ ID NO: 52 - protein, 14 aa, Homo sapiens
VTGEGCVYLQTSLK

SEQ ID NO: 53 - protein, 12 aa, Homo sapiens
FVVLFNPLEQER

SEQ ID NO: 54 - protein, 14 aa, Homo sapiens
VIDSGTSDFALSNR

SEQ ID NO: 55 - protein, 15 aa, Homo sapiens
EYCGVPGDGDEELLR

SEQ ID NO: 56 - protein, 15 aa, Homo sapiens
CVLFPYGGCQGNGNK

SEQ ID NO: 57 - protein, 17 aa, Homo sapiens
GECVPGEQEPEPILIPR

SEQ ID NO: 58 - protein, 15 aa, Homo sapiens
VAEEIQDEVDELLQK

SEQ ID NO: 59 - protein, 16 aa, Homo sapiens
YLETPGDENEHAHFQK

SEQ ID NO: 60 - protein, 17 aa, Homo sapiens
EQNYSDDVLANMISEPR

SEQ ID NO: 61 - protein, 16 aa, Homo sapiens
ISYGNDALMPSLTETK

SEQ ID NO: 62 - protein, 13 aa, Homo sapiens
FQVHTHLQVIEER

SEQ ID NO: 63 - protein, 10 aa, Homo sapiens
QMYPELQIAR

SEQ ID NO: 64 - protein, 19 aa, Homo sapiens
AALEGFLAALQADPPQAER

SEQ ID NO: 65 - protein, 13 aa, Homo sapiens
AALEGFLAALQAD

SEQ ID NO: 66 - protein, 12 aa, Homo sapiens
HYQHVAAVDPEK

SEQ ID NO: 67 - protein, 9 aa, Homo sapiens
MNPLEQYER

SEQ ID NO: 68 - protein, 15 aa, Homo sapiens
AVYDQSATALHFLGR

SEQ ID NO: 69 - protein, 12 aa, Homo sapiens
DQSATALHFLGR

SEQ ID NO: 70 - protein, 12 aa, Homo sapiens
LIQPHYASDLDK

SEQ ID NO: 71 - protein, 10 aa, Homo sapiens
ALYTPVVLPR

SEQ ID NO: 72 - protein, 10 aa, Homo sapiens
ANRPFLVFIR

SEQ ID NO: 73 - protein, 7 aa, Homo sapiens
PLGEEMR

SEQ ID NO: 74 - protein, 8 aa, Homo sapiens
AELQEGAR

SEQ ID NO: 75 - protein, 11 aa, Homo sapiens
THLAPYSDELR

SEQ ID NO: 76 - protein, 17 aa, Homo sapiens
DSGRDYVSQFEGSALGK

SEQ ID NO: 77 - protein, 13 aa, Homo sapiens
VKDLATVYVDVLK

SEQ ID NO: 78 - protein, 12 aa, Homo sapiens
VSFLSALEEYTK

SEQ ID NO: 79 - protein, 14 aa, Homo sapiens
LGPVTQEFWDNLEK

SEQ ID NO: 80 - protein, 13 aa, Homo sapiens
DYVSQFEGSALGK

SEQ ID NO: 81 - protein, 14 aa, Homo sapiens
LLDNWDSVTSTFSK

SEQ ID NO: 82 - protein, 9 aa, Homo sapiens
WQEEMELYR

SEQ ID NO: 83 - protein, 16 aa, Homo sapiens
EQLGPVTQEFWDNLEK

SEQ ID NO: 84 - protein, 11 aa, Homo sapiens
QGLLPVLESFK

SEQ ID NO: 85 - protein, 11 aa, Homo sapiens
VKSPELQAEAK

SEQ ID NO: 86 - protein, 9 aa, Homo sapiens
SPELQAEAK

SEQ ID NO: 87 - protein, 9 aa, Homo sapiens
FQTVTDYGK

SEQ ID NO: 88 - protein, 20 aa, Homo sapiens
EPCVESLVSQYFQTVTDYGK

SEQ ID NO: 89 - protein, 17 aa, Homo sapiens
SLAELGGHLDQQVEEFR

SEQ ID NO: 90 - protein, 11 aa, Homo sapiens
DKVNSFFSTFK

SEQ ID NO: 91 - protein, 13 aa, Homo sapiens
LGEVNTYAGDLQK

SEQ ID NO: 92 - protein, 11 aa, Homo sapiens
LLPHANEVSQK

SEQ ID NO: 93 - protein, 16 aa, Homo sapiens
DALSSVQESQVAQQAR

SEQ ID NO: 94 - protein, 11 aa, Homo sapiens
MTVTDQVNCPK

SEQ ID NO: 95 - protein, 14 aa, Homo sapiens
CPNPPVQENFDVNK

SEQ ID NO: 96 - protein, 10 aa, Homo sapiens
ILTSNNIDVK

SEQ ID NO: 97 - protein, 12 aa, Homo sapiens
NPPVQENFDVNK

SEQ ID NO: 98 - protein, 8 aa, Homo sapiens
QWAGLVEK

SEQ ID NO: 99 - protein, 15 aa, Homo sapiens
GEVQAMLGQSTEELR

SEQ ID NO: 100 - protein, 15 aa, Homo sapiens
KVEQAVETEPEPELR

SEQ ID NO: 101 - protein, 8 aa, Homo sapiens
ALMDETMK

SEQ ID NO: 102 - protein, 9 aa, Homo sapiens
LEEQAQQIR

SEQ ID NO: 103 - protein, 15 aa, Homo sapiens
SCNTGFYLNGADSAK

SEQ ID NO: 104 - protein, 17 aa, Homo sapiens
TCPKPDDLPFSTVVPLK

SEQ ID NO: 105 - protein, 14 aa, Homo sapiens
VCPFAGILENGAVR

SEQ ID NO: 106 - protein, 10 aa, Homo sapiens
NQEACELSNN

SEQ ID NO: 107 - protein, 18 aa, Homo sapiens
GSVYIGGAPDVATLTGGR

SEQ ID NO: 108 - protein, 11 aa, Homo sapiens
SPGPNVAVNAK

SEQ ID NO: 109 - protein, 9 aa, Homo sapiens
ATVVYQGER

SEQ ID NO: 110 - protein, 10 aa, Homo sapiens
AGILENGAVR

SEQ ID NO: 111 - protein, 10 aa, Homo sapiens
VNHVTLSQPK

SEQ ID NO: 112 - protein, 10 aa, Homo sapiens
SHLIIAQVAK

SEQ ID NO: 113 - protein, 9 aa, Homo sapiens
SSELKTACR

SEQ ID NO: 114 - protein, 24 aa, Homo sapiens
AEPPKAPEQEQAAPGPAAGGEAPK

SEQ ID NO: 115 - protein, 11 aa, Homo sapiens
DIQGSLQDIFK

SEQ ID NO: 116 - protein, 12 aa, Homo sapiens
EDLDCTPGFQQK

SEQ ID NO: 117 - protein, 10 aa, Homo sapiens
DGNKGHKQIR

SEQ ID NO: 118 - protein, 8 aa, Homo sapiens
KSLEELSK

SEQ ID NO: 119 - protein, 15 aa, Homo sapiens
IHGQNVPFDAVVVDK

SEQ ID NO: 120 - protein, 13 aa, Homo sapiens
EGLDLQVLEDSGR

SEQ ID NO: 121 - protein, 11 aa, Homo sapiens
ISLSGVHHFAR

SEQ ID NO: 122 - protein, 11 aa, Homo sapiens
IPDGVVSVSPK

SEQ ID NO: 123 - protein, 7 aa, Homo sapiens
QYDSILR

SEQ ID NO: 124 - protein, 11 aa, Homo sapiens
AMQHISYLNSR

SEQ ID NO: 125 - protein, 25 aa, Homo sapiens
GVQIQAHPSQLVLTLEGEDLGELDK

SEQ ID NO: 126 - protein, 13 aa, Homo sapiens
LQQEDGISFEYHR

SEQ ID NO: 127 - protein, 13 aa, Homo sapiens
IANIDDTQYLAAK

SEQ ID NO: 128 - protein, 17 aa, Homo sapiens
QGNAIGVTACDIDGDGR

SEQ ID NO: 129 - protein, 10 aa, Homo sapiens
DVAAEAGVSK

SEQ ID NO: 130 - protein, 10 aa, Homo sapiens
ALFLETEQLK

SEQ ID NO: 131 - protein, 9 aa, Homo sapiens
FAGVFHVEK

SEQ ID NO: 132 - protein, 10 aa, Homo sapiens
ALSIGFETCR

SEQ ID NO: 133 - protein, 12 aa, Homo sapiens
DFDDFNLEDAVK

SEQ ID NO: 134 - protein, 19 aa, Homo sapiens
VNGSPVDNHPFAGDVVFPR

SEQ ID NO: 135 - protein, 11 aa, Homo sapiens
DGNPFYFTDHR

SEQ ID NO: 136 - protein, 10 aa, Homo sapiens
VEEVKPLEGR

SEQ ID NO: 137 - protein, 12 aa, Homo sapiens
NIELEHIEQDER

SEQ ID NO: 138 - protein, 25 aa, Homo sapiens
GAGPESEPYIFQTPEGVPEQPTFLK

SEQ ID NO: 139 - protein, 9 aa, Homo sapiens
VIAVNEVGR

SEQ ID NO: 140 - protein, 17 aa, Homo sapiens
GEAGGQAEAEGDAPGPR

SEQ ID NO: 141 - protein, 14 aa, Homo sapiens
CLVVCDSNPATDSK

SEQ ID NO: 142 - protein, 14 aa, Homo sapiens
EVGPTNADPVCLAK

SEQ ID NO: 143 - protein, 14 aa, Homo sapiens
DLYSGLIGPLIVCR

SEQ ID NO: 144 - protein, 19 aa, Homo sapiens
LLATEEQSPGEGDGNCVTR

SEQ ID NO: 145 - protein, 20 aa, Homo sapiens
MLLATEEQSPGEGDGNCVTR

SEQ ID NO: 146 - protein, 23 aa, Homo sapiens
GVYSSDVFDIFPGTYQTLEMFPR

SEQ ID NO: 147 - protein, 21 aa, Homo sapiens
MFTTAPDQVDKEDEDFQESNK

SEQ ID NO: 148 - protein, 14 aa, Homo sapiens
DIASGLIGPLIICK

SEQ ID NO: 149 - protein, 7 aa, Homo sapiens
YTVNQCR

SEQ ID NO: 150 - protein, 13 aa, Homo sapiens
EGDGSCFPDALDR

SEQ ID NO: 151 - protein, 12 aa, Homo sapiens
QLLLSAALSAGK

SEQ ID NO: 152 - protein, 17 aa, Homo sapiens
ALDLDDFQGSFCGQDLR

SEQ ID NO: 153 - protein, 14 aa, Homo sapiens
TLLSVGGWNFGSQR

SEQ ID NO: 154 - protein, 12 aa, Homo sapiens
PQALPEPMQESK

SEQ ID NO: 155 - protein, 9 aa, Homo sapiens
VAHQLQALR

SEQ ID NO: 156 - protein, 9 aa, Homo sapiens
EAVEEPSSK

SEQ ID NO: 157 - protein, 20 aa, Homo sapiens
YPGPQAEGDSEGLSQGLVDR

SEQ ID NO: 158 - protein, 19 aa, Homo sapiens
SEALAVDGAGKPGAEEAQD

SEQ ID NO: 159 - protein, 23 aa, Homo sapiens
SEALAVDGAGKPGAEEAQDPEGK

SEQ ID NO: 160 - protein, 31 aa, Homo sapiens
AEGNNQAPGEEEEEEEEATNTHPPASLPSQK

SEQ ID NO: 161 - protein, 9 aa, Homo sapiens
WPITILSFR

SEQ ID NO: 162 - protein, 14 aa, Homo sapiens
TQSSLVPALTDFVR

SEQ ID NO: 163 - protein, 10 aa, Homo sapiens
EILSVDCSTN

SEQ ID NO: 164 - protein, 9 aa, Homo sapiens
IDSLLENDR

SEQ ID NO: 165 - protein, 15 aa, Homo sapiens
QQTHMLDVMQDHFSR

SEQ ID NO: 166 - protein, 16 aa, Homo sapiens
EILSVDCSTNNPSQAK

SEQ ID NO: 167 - protein, 27 aa, Homo sapiens
LQEDADGSCALLSPYVQPVCLPSGAAR

SEQ ID NO: 168 - protein, 7 aa, Homo sapiens
PGLSLVK

SEQ ID NO: 169 - protein, 12 aa, Homo sapiens
TNQVNSGGVLLR

SEQ ID NO: 170 - protein, 18 aa, Homo sapiens
FNAVLTNPQGDYDTSTGK

SEQ ID NO: 171 - protein, 8 aa, Homo sapiens
SDFSNEER

SEQ ID NO: 172 - protein, 16 aa, Homo sapiens
NQGILEFYGDDIALLK

SEQ ID NO: 173 - protein, 15 aa, Homo sapiens
CPAPVSFENGIYTPR

SEQ ID NO: 174 - protein, 18 aa, Homo sapiens
KVEGTAFVIFGIQDGEQR

SEQ ID NO: 175 - protein, 25 aa, Homo sapiens
EGVQKEDIPPADLSDQVPDTESETR

SEQ ID NO: 176 - protein, 16 aa, Homo sapiens
VHQYFNVELIQPGAVK

SEQ ID NO: 177 - protein, 14 aa, Homo sapiens
AGDFLEANYMNLQR

SEQ ID NO: 178 - protein, 10 aa, Homo sapiens
QLYNVEATSY

SEQ ID NO: 179 - protein, 11 aa, Homo sapiens
QELSEAEQATR

SEQ ID NO: 180 - protein, 11 aa, Homo sapiens
NVELIQPGAVK

SEQ ID NO: 181 - protein, 12 aa, Homo sapiens
KQELSEAEQATR

SEQ ID NO: 182 - protein, 14 aa, Homo sapiens
KVFLDCCNYITELR

SEQ ID NO: 183 - protein, 9 aa, Homo sapiens
FISLGEACK

SEQ ID NO: 184 - protein, 11 aa, Homo sapiens
ACEPGVDYVYK

SEQ ID NO: 185 - protein, 24 aa, Homo sapiens
DYAGVFSDAGLTFTSSSGQQTAQR

SEQ ID NO: 186 - protein, 15 aa, Homo sapiens
SEFPESWLWNVEDLK

SEQ ID NO: 187 - protein, 22 aa, Homo sapiens
VPVAVQGEDTVQSLTQGDGVAK

SEQ ID NO: 188 - protein, 18 aa, Homo sapiens
SGQSEDRQPVPGQQMTLK

SEQ ID NO: 189 - protein, 11 aa, Homo sapiens
QPSSAFAAFVK

SEQ ID NO: 190 - protein, 14 aa, Homo sapiens
GQGTLSVVTMYHAK

SEQ ID NO: 191 - protein, 20 aa, Homo sapiens
ILLQGTPVAQMTEDAVDAER

SEQ ID NO: 192 - protein, 10 aa, Homo sapiens
DSCVGSLVVK

SEQ ID NO: 193 - protein, 13 aa, Homo sapiens
VFLDCCNYITELR

SEQ ID NO: 194 - protein, 7 aa, Homo sapiens
NEQVEIR

SEQ ID NO: 195 - protein, 12 aa, Homo sapiens
SGSDEVQVGQQR

SEQ ID NO: 196 - protein, 15 aa, Homo sapiens
VIAVHYLDETEQWEK

SEQ ID NO: 197 - protein, 16 aa, Homo sapiens
DICEEQVNSLPGSITK

SEQ ID NO: 198 - protein, 12 aa, Homo sapiens
APSTWLTAYVVK

SEQ ID NO: 199 - protein, 19 aa, Homo sapiens
VQLSNDFDEYIMAIEQTIK

SEQ ID NO: 200 - protein, 11 aa, Homo sapiens
VTIKPAPETEK

SEQ ID NO: 201 - protein, 12 aa, Homo sapiens
DSSTWLTAFVLK

SEQ ID NO: 202 - protein, 22 aa, Homo sapiens
ASAGLLGAHAAAITAYALTLTK

SEQ ID NO: 203 - protein, 20 aa, Homo sapiens
AACAQLNDFLQEYGTQGCQV

SEQ ID NO: 204 - protein, 16 aa, Homo sapiens
ALVEGVDQLFTDYQIK

SEQ ID NO: 205 - protein, 13 aa, Homo sapiens
AFTECCVVASQLR

SEQ ID NO: 206 - protein, 11 aa, Homo sapiens
IGESIELTCPK

SEQ ID NO: 207 - protein, 10 aa, Homo sapiens
AMAVEDIISR

SEQ ID NO: 208 - protein, 14 aa, Homo sapiens
LEQGENVFLQATDK

SEQ ID NO: 209 - protein, 9 aa, Homo sapiens
GGGALLGDR

SEQ ID NO: 210 - protein, 15 aa, Homo sapiens
FCGQLGSPLGNPPGK

SEQ ID NO: 211 - protein, 11 aa, Homo sapiens
YTTTMGVNTYK

SEQ ID NO: 212 - protein, 9 aa, Homo sapiens
SCGLHQLLR

SEQ ID NO: 213 - protein, 20 aa, Homo sapiens
LLSQQQADGSFQDLSPVIHR

SEQ ID NO: 214 - protein, 17 aa, Homo sapiens
GHLFLQTDQPIYNPGQR

SEQ ID NO: 215 - protein, 15 aa, Homo sapiens
VLSLAQEQVGGSPEK

SEQ ID NO: 216 - protein, 23 aa, Homo sapiens
TLEIPGNSDPNMIPDGDFNSYVR

SEQ ID NO: 217 - protein, 13 aa, Homo sapiens
AEMADQAAAWLTR

SEQ ID NO: 218 - protein, 12 aa, Homo sapiens
LNMGITDLQGLR

SEQ ID NO: 219 - protein, 13 aa, Homo sapiens
LSGNVLSYTFQVK

SEQ ID NO: 220 - protein, 14 aa, Homo sapiens
GEVLDNSFTGGICK

SEQ ID NO: 221 - protein, 21 aa, Homo sapiens
SCVGETTESTQCEDEELEHLR

SEQ ID NO: 222 - protein, 17 aa, Homo sapiens
YGFCEAADQFHVLDEVR

SEQ ID NO: 223 - protein, 17 aa, Homo sapiens
AEQCCEETASSISLHGK

SEQ ID NO: 224 - protein, 13 aa, Homo sapiens
PQGSCSLEGVEIK

SEQ ID NO: 225 - protein, 17 aa, Homo sapiens
KEAGIPEFYDYDVALIK

SEQ ID NO: 226 - protein, 9 aa, Homo sapiens
DISEVVTPR

SEQ ID NO: 227 - protein, 10 aa, Homo sapiens
DAQYAPGYDK

SEQ ID NO: 228 - protein, 17 aa, Homo sapiens
FLCTGGVSPYADPNTCR

SEQ ID NO: 229 - protein, 24 aa, Homo sapiens
LLQEGQALEYVCPSGFYPYPVQTR

SEQ ID NO: 230 - protein, 16 aa, Homo sapiens
EAGIPEFYDYDVALIK

SEQ ID NO: 231 - protein, 11 aa, Homo sapiens
LEDSVTYHCSR

SEQ ID NO: 232 - protein, 19 aa, Homo sapiens
GDAVCTESGWRPLPSCEEK

SEQ ID NO: 233 - protein, 18 aa, Homo sapiens
LGYVTADGETSGSITCGK

SEQ ID NO: 234 - protein, 13 aa, Homo sapiens
WSSPPQCEGLPCK

SEQ ID NO: 235 - protein, 10 aa, Homo sapiens
TGDEITYQCR

SEQ ID NO: 236 - protein, 16 aa, Homo sapiens
SCDNPYIPNGDYSPLR

SEQ ID NO: 237 - protein, 14 aa, Homo sapiens
CFEGFGIDGPAIAK

SEQ ID NO: 238 - protein, 18 aa, Homo sapiens
EQVQSCGPPPELLNGNVK

SEQ ID NO: 239 - protein, 13 aa, Homo sapiens
TLTGGNVFEYGVK

SEQ ID NO: 240 - protein, 18 aa, Homo sapiens
DTSCVNPPTVQNAYIVSR

SEQ ID NO: 241 - protein, 12 aa, Homo sapiens
SSNLIILEEHLK

SEQ ID NO: 242 - protein, 13 aa, Homo sapiens
AQLGDLPWQVAIK

SEQ ID NO: 243 - protein, 14 aa, Homo sapiens
NDGGIYTCFAENNR

SEQ ID NO: 244 - protein, 23 aa, Homo sapiens
GFGPIFEEQPINTIYPEESLEGK

SEQ ID NO: 245 - protein, 9 aa, Homo sapiens
DGEYVVEVR

SEQ ID NO: 246 - protein, 15 aa, Homo sapiens
YSMVGGNLVINNPDK

SEQ ID NO: 247 - protein, 10 aa, Homo sapiens
VTVTNPDTGR

SEQ ID NO: 248 - protein, 11 aa, Homo sapiens
KVTVTNPDTGR

SEQ ID NO: 249 - protein, 14 aa, Homo sapiens
AHSDGGDGVVSQVK

SEQ ID NO: 250 - protein, 8 aa, Homo sapiens
IVESYQIR

SEQ ID NO: 251 - protein, 8 aa, Homo sapiens
LATVSTHK

SEQ ID NO: 252 - protein, 10 aa, Homo sapiens
NKTENGLTPK

SEQ ID NO: 253 - protein, 16 aa, Homo sapiens
LVGGPMDASVEEEGVR

SEQ ID NO: 254 - protein, 11 aa, Homo sapiens
VVQCSDLGLDK

SEQ ID NO: 255 - protein, 10 aa, Homo sapiens
ELEPDGALDR

SEQ ID NO: 256 - protein, 13 aa, Homo sapiens
GYYYEIPSIGAIR

SEQ ID NO: 257 - protein, 7 aa, Homo sapiens
EQLELLQ

SEQ ID NO: 258 - protein, 13 aa, Homo sapiens
AFYSEQPDFSGHK

SEQ ID NO: 259 - protein, 13 aa, Homo sapiens
CFELQEAGPPDCR

SEQ ID NO: 260 - protein, 11 aa, Homo sapiens
RIEDIHLLVER

SEQ ID NO: 261 - protein, 13 aa, Homo sapiens
CVNHYGGYLCLPK

SEQ ID NO: 262 - protein, 18 aa, Homo sapiens
IDTIAADESFTQVDIGDR

SEQ ID NO: 263 - protein, 16 aa, Homo sapiens
FLPGQGLVLYPQIGDK

SEQ ID NO: 264 - protein, 11 aa, Homo sapiens
SEGGFTATGQR

SEQ ID NO: 265 - protein, 16 aa, Homo sapiens
FSCFQEEAPQPHYQLR

SEQ ID NO: 266 - protein, 12 aa, Homo sapiens
ASEGGFTATGQR

SEQ ID NO: 267 - protein, 9 aa, Homo sapiens
LVWEEAMSR

SEQ ID NO: 268 - protein, 14 aa, Homo sapiens
NPCQDPYILTPENR

SEQ ID NO: 269 - protein, 10 aa, Homo sapiens
LNCEDIDECR

SEQ ID NO: 270 - protein, 9 aa, Homo sapiens
VGASDGVFR

SEQ ID NO: 271 - protein, 9 aa, Homo sapiens
GSQAVSYTR

SEQ ID NO: 272 - protein, 9 aa, Homo sapiens
TPDGSLLVR

SEQ ID NO: 273 - protein, 13 aa, Homo sapiens
RQSEDSTFYLGER

SEQ ID NO: 274 - protein, 17 aa, Homo sapiens
TTIEKPVWLGFLGPIIK

SEQ ID NO: 275 - protein, 19 aa, Homo sapiens
MHSMNGFMYGNQPGLTMCK

SEQ ID NO: 276 - protein, 22 aa, Homo sapiens
MYSVNGYTFGSLPGLSMCAEDR

SEQ ID NO: 277 - protein, 11 aa, Homo sapiens
EYTDASFTNRK

SEQ ID NO: 278 - protein, 16 aa, Homo sapiens
NNEGTYYSPNYNPQSR

SEQ ID NO: 279 - protein, 20 aa, Homo sapiens
AEEEHLGILGPQLHADVGDK

SEQ ID NO: 280 - protein, 8 aa, Homo sapiens
QLEQVIAK

SEQ ID NO: 281 - protein, 15 aa, Homo sapiens
DNENVVNEYSSELEK

SEQ ID NO: 282 - protein, 9 aa, Homo sapiens
DNCCILDER

SEQ ID NO: 283 - protein, 18 aa, Homo sapiens
EESPLLIGQQSTVSDVPR

SEQ ID NO: 284 - protein, 11 aa, Homo sapiens
DCSLPYATESK

SEQ ID NO: 285 - protein, 20 aa, Homo sapiens
ITYYHLSFPTNIQAPAVVFR

SEQ ID NO: 286 - protein, 17 aa, Homo sapiens
SQETGDLDVGGLQETDK

SEQ ID NO: 287 - protein, 10 aa, Homo sapiens
MCVDVNECQR

SEQ ID NO: 288 - protein, 12 aa, Homo sapiens
PYQYPALTPEQK

SEQ ID NO: 289 - protein, 16 aa, Homo sapiens
FLGWDDDYWSVDPLDR

SEQ ID NO: 290 - protein, 22 aa, Homo sapiens
AQPVQVAEGSEPDGFWEALGGK

SEQ ID NO: 291 - protein, 10 aa, Homo sapiens
EVQGFESATF

SEQ ID NO: 292 - protein, 11 aa, Homo sapiens
SEDCFILDHGK

SEQ ID NO: 293 - protein, 9 aa, Homo sapiens
KGGVASGFK

SEQ ID NO: 294 - protein, 20 aa, Homo sapiens
SVVLIPLGAVDDGEHSQNEK

SEQ ID NO: 295 - protein, 8 aa, Homo sapiens
FLEMAQLH

SEQ ID NO: 296 - protein, 20 aa, Homo sapiens
DQLVIPDGQEEEQEAAGEGR

SEQ ID NO: 297 - protein, 9 aa, Homo sapiens
DTINLLDQR

SEQ ID NO: 298 - protein, 16 aa, Homo sapiens
NVAEGETKPDPDVTER

SEQ ID NO: 299 - protein, 15 aa, Homo sapiens
GECQAEGVLFFQGDR

SEQ ID NO: 300 - protein, 30 aa, Homo sapiens
SLGPNSCSANGPGLYLIHGPNLYCYSDVEK

SEQ ID NO: 301 - protein, 21 aa, Homo sapiens
LLQDEFPGIPSPLDAAVECHR

SEQ ID NO: 302 - protein, 11 aa, Homo sapiens
YYCFQGNQFLR

SEQ ID NO: 303 - protein, 15 aa, Homo sapiens
LIHGPNLYCYSDVEK

SEQ ID NO: 304 - protein, 12 aa, Homo sapiens
EWFWDLATGTMK

SEQ ID NO: 305 - protein, 10 aa, Homo sapiens
QGHNSVFLIK

SEQ ID NO: 306 - protein, 19 aa, Homo sapiens
CSPHLVLSALTSDNHGATY

SEQ ID NO: 307 - protein, 16 aa, Homo sapiens
SGAQATWTELPWPHEK

SEQ ID NO: 308 - protein, 24 aa, Homo sapiens
EVGTPHGIILDSVDAAFICPGSSR

SEQ ID NO: 309 - protein, 11 aa, Homo sapiens
GGYTLVSGYPK

SEQ ID NO: 310 - protein, 11 aa, Homo sapiens
ASNVEINEGAR

SEQ ID NO: 311 - protein, 21 aa, Homo sapiens
DALENIDPATQMMILNCIYFK

SEQ ID NO: 312 - protein, 13 aa, Homo sapiens
SGGCFWDNGHLYR

SEQ ID NO: 313 - protein, 15 aa, Homo sapiens
YGSGGCFWDNGHLYR

SEQ ID NO: 314 - protein, 10 aa, Homo sapiens
VLALLDVPDK

SEQ ID NO: 315 - protein, 9 aa, Homo sapiens
SWLQQEQER

SEQ ID NO: 316 - protein, 10 aa, Homo sapiens
WDEELAAFAK

SEQ ID NO: 317 - protein, 13 aa, Homo sapiens
HGNTDSEGIVEVK

SEQ ID NO: 318 - protein, 12 aa, Homo sapiens
TFTCTAAYPESK

SEQ ID NO: 319 - protein, 20 aa, Homo sapiens
CPAPELLGGPSVFLFPPKPK

SEQ ID NO: 320 - protein, 10 aa, Homo sapiens
SLSSTLTLSK

SEQ ID NO: 321 - protein, 9 aa, Homo sapiens
FQLEEFSPR

SEQ ID NO: 322 - protein, 9 aa, Homo sapiens
VWSVASTVR

SEQ ID NO: 323 - protein, 9 aa, Homo sapiens
IHASQAVVR

SEQ ID NO: 324 - protein, 17 aa, Homo sapiens
EQAVEGGEVELSCLVPR

SEQ ID NO: 325 - protein, 10 aa, Homo sapiens
DGGELPDPDR

SEQ ID NO: 326 - protein, 10 aa, Homo sapiens
DTQFSYAVFK

SEQ ID NO: 327 - protein, 8 aa, Homo sapiens
YDLSALVR

SEQ ID NO: 328 - protein, 13 aa, Homo sapiens
TPCQQELDQVLER

SEQ ID NO: 329 - protein, 10 aa, Homo sapiens
APAVAEENPK

SEQ ID NO: 330 - protein, 13 aa, Homo sapiens
GAQTLYVPNCDHR

SEQ ID NO: 331 - protein, 8 aa, Homo sapiens
NVVFVIDK

SEQ ID NO: 332 - protein, 18 aa, Homo sapiens
FAHYVVTSQVVNTANEAR

SEQ ID NO: 333 - protein, 11 aa, Homo sapiens
LSNENHGIAQR

SEQ ID NO: 334 - protein, 10 aa, Homo sapiens
SSALDMENFR

SEQ ID NO: 335 - protein, 9 aa, Homo sapiens
LGVYELLLK

SEQ ID NO: 336 - protein, 13 aa, Homo sapiens
VTSDHINFDPVYK

SEQ ID NO: 337 - protein, 21 aa, Homo sapiens
CYLITVTPVYADGPGSPESIK

SEQ ID NO: 338 - protein, 10 aa, Homo sapiens
ILDYEVTLTR

SEQ ID NO: 339 - protein, 12 aa, Homo sapiens
LSELIQPLPLER

SEQ ID NO: 340 - protein, 20 aa, Homo sapiens
AVIHPDYDAASHDQDIMLLR

SEQ ID NO: 341 - protein, 8 aa, Homo sapiens
IEISELNR

SEQ ID NO: 342 - protein, 17 aa, Homo sapiens
QESQSEEIDCNDKDLFK

SEQ ID NO: 343 - protein, 14 aa, Homo sapiens
IASFSQNCDIYPGK

SEQ ID NO: 344 - protein, 15 aa, Homo sapiens
YNSQNQSNNQFVLYR

SEQ ID NO: 345 - protein, 11 aa, Homo sapiens
ENFLFLTPDCK

SEQ ID NO: 346 - protein, 12 aa, Homo sapiens
AATGECTATVGK

SEQ ID NO: 347 - protein, 19 aa, Homo sapiens
DIPTNSPELEETLTHTITK

SEQ ID NO: 348 - protein, 13 aa, Homo sapiens
VTVNYPPTITESK

SEQ ID NO: 349 - protein, 10 aa, Homo sapiens
SGFSLGSDGK

SEQ ID NO: 350 - protein, 10 aa, Homo sapiens
NNQIDHIDEK

SEQ ID NO: 351 - protein, 11 aa, Homo sapiens
SLEDLQLTHNK

SEQ ID NO: 352 - protein, 8 aa, Homo sapiens
IPLNDLFR

SEQ ID NO: 353 - protein, 9 aa, Homo sapiens
IDITLSSVK

SEQ ID NO: 354 - protein, 9 aa, Homo sapiens
ETVIIPNEK

SEQ ID NO: 355 - protein, 11 aa, Homo sapiens
EKETVIIPNEK

SEQ ID NO: 356 - protein, 19 aa, Homo sapiens
AFPALTSLDLSDNPGLGER

SEQ ID NO: 357 - protein, 21 aa, Homo sapiens
ITGTMPPLPLEATGLALSSLR

SEQ ID NO: 358 - protein, 12 aa, Homo sapiens
FSDEGGFTCFFR

SEQ ID NO: 359 - protein, 7 aa, Homo sapiens
LSTFTQF

SEQ ID NO: 360 - protein, 16 aa, Homo sapiens
TALASGGVLDASGDYR

SEQ ID NO: 361 - protein, 9 aa, Homo sapiens
EPGEFALLR

SEQ ID NO: 362 - protein, 10 aa, Homo sapiens
WEGPLSVSVF

SEQ ID NO: 363 - protein, 8 aa, Homo sapiens
SCQEVFDK

SEQ ID NO: 364 - protein, 20 aa, Homo sapiens
DGSPDVTTADIGANTPDATK

SEQ ID NO: 365 - protein, 10 aa, Homo sapiens
AVGEEVWHSK

SEQ ID NO: 366 - protein, 13 aa, Homo sapiens
YIFSDDSSQLTIK

SEQ ID NO: 367 - protein, 13 aa, Homo sapiens
NDEAEYICIAENK

SEQ ID NO: 368 - protein, 11 aa, Homo sapiens
AGEQDATIHLK

SEQ ID NO: 369 - protein, 10 aa, Homo sapiens
IEIFQTLPVR

SEQ ID NO: 370 - protein, 11 aa, Homo sapiens
LPDLIADALHR

SEQ ID NO: 371 - protein, 9 aa, Homo sapiens
MGSISFDFR

SEQ ID NO: 372 - protein, 11 aa, Homo sapiens
LPDLINDALHR

SEQ ID NO: 373 - protein, 13 aa, Homo sapiens
ELGGEVFYVGPAR

SEQ ID NO: 374 - protein, 13 aa, Homo sapiens
YALTFAEAQEACR

SEQ ID NO: 375 - protein, 11 aa, Homo sapiens
YPGSVNSAVLR

SEQ ID NO: 376 - protein, 20 aa, Homo sapiens
TLQIIHVSEADSGNYQCIAK

SEQ ID NO: 377 - protein, 13 aa, Homo sapiens
NALGAIHHTISVR

SEQ ID NO: 378 - protein, 9 aa, Homo sapiens
QPEYAVVQR

SEQ ID NO: 379 - protein, 16 aa, Homo sapiens
VFNTPEGVPSAPSSLK

SEQ ID NO: 380 - protein, 8 aa, Homo sapiens
VSISTLNK

SEQ ID NO: 381 - protein, 14 aa, Homo sapiens
FDATQAFVGDIAQF

SEQ ID NO: 382 - protein, 18 aa, Homo sapiens
AVGFVSEDEYLEIQGITR

SEQ ID NO: 383 - protein, 11 aa, Homo sapiens
APAAHPEGQLK

SEQ ID NO: 384 - protein, 11 aa, Homo sapiens
SLEVLNLSSNK

SEQ ID NO: 385 - protein, 20 aa, Homo sapiens
DQSGEYECSALNDVAAPDVR

SEQ ID NO: 386 - protein, 14 aa, Homo sapiens
NLEHLYLQNNEIEK

SEQ ID NO: 387 - protein, 21 aa, Homo sapiens
QLYNKYPDAVATWLNPDPSQK

SEQ ID NO: 388 - protein, 20 aa, Homo sapiens
KANDESNEHSDVIDSQELSK

SEQ ID NO: 389 - protein, 8 aa, Homo sapiens
SSSVWEEK

SEQ ID NO: 390 - protein, 13 aa, Homo sapiens
TDCPGDALFDLLR

SEQ ID NO: 391 - protein, 8 aa, Homo sapiens
NNLVIFHR

SEQ ID NO: 392 - protein, 14 aa, Homo sapiens
LYTLVLTDPDAPSR

SEQ ID NO: 393 - protein, 11 aa, Homo sapiens
AVEPQLQEEER

SEQ ID NO: 394 - protein, 10 aa, Homo sapiens
AIINLAVYGK

SEQ ID NO: 395 - protein, 10 aa, Homo sapiens
FQPTLLTLPR

SEQ ID NO: 396 - protein, 11 aa, Homo sapiens
TNLESILSYPK

SEQ ID NO: 397 - protein, 8 aa, Homo sapiens
TLYSSSPR

SEQ ID NO: 398 - protein, 15 aa, Homo sapiens
IKVTTSQDMLSIMEK

SEQ ID NO: 399 - protein, 13 aa, Homo sapiens
KYPVAHFIDQTLK

SEQ ID NO: 400 - protein, 17 aa, Homo sapiens
GVTSVSQIFHSPDLAIR

SEQ ID NO: 401 - protein, 14 aa, Homo sapiens
LEDMEQALSPSVFK

SEQ ID NO: 402 - protein, 13 aa, Homo sapiens
VTTSQDMLSIMEK

SEQ ID NO: 403 - protein, 18 aa, Homo sapiens
LLNLDGTCADSYSFVFSR

SEQ ID NO: 404 - protein, 16 aa, Homo sapiens
LLNLDGTCADSYSFVF

SEQ ID NO: 405 - protein, 10 aa, Homo sapiens
YWGVASFLQK

SEQ ID NO: 406 - protein, 18 aa, Homo sapiens
NPDADKGPWCFTTDPSVR

SEQ ID NO: 407 - protein, 19 aa, Homo sapiens
EPLDDYVNTQGASLFSVTK

SEQ ID NO: 408 - protein, 13 aa, Homo sapiens
QLGAGSIEECAAK

SEQ ID NO: 409 - protein, 17 aa, Homo sapiens
FSPATHPSEGLEENYCR

SEQ ID NO: 410 - protein, 12 aa, Homo sapiens
SHSGGELESLAR

SEQ ID NO: 411 - protein, 16 aa, Homo sapiens
DAEEDDSLANSSDLLK

SEQ ID NO: 412 - protein, 19 aa, Homo sapiens
ELLQLSKPELPQDGTSTLR

SEQ ID NO: 413 - protein, 9 aa, Homo sapiens
AVEIEEQTK

SEQ ID NO: 414 - protein, 10 aa, Homo sapiens
PDAPAAQLAR

SEQ ID NO: 415 - protein, 31 aa, Homo sapiens
PRPPVYDDGPAGPDAEEAGDETPDVDPELLR

SEQ ID NO: 416 - protein, 21 aa, Homo sapiens
SVSVVETDYDQYALLYSQGSK

SEQ ID NO: 417 - protein, 11 aa, Homo sapiens
VSVQPNFQQDK

SEQ ID NO: 418 - protein, 16 aa, Homo sapiens
APEAQVSVQPNFQQDK

SEQ ID NO: 419 - protein, 16 aa, Homo sapiens
TMLLQPAGSLGSYSYR

SEQ ID NO: 420 - protein, 12 aa, Homo sapiens
QVSVQPNFQQDK

SEQ ID NO: 421 - protein, 18 aa, Homo sapiens
VVETDYDQYALLYSQGSK

SEQ ID NO: 422 - protein, 7 aa, Homo sapiens
GPGEDFR

SEQ ID NO: 423 - protein, 10 aa, Homo sapiens
DFRMATLYSR

SEQ ID NO: 424 - protein, 7 aa, Homo sapiens
AALSMCK

SEQ ID NO: 425 - protein, 8 aa, Homo sapiens
SPFEQHIK

SEQ ID NO: 426 - protein, 10 aa, Homo sapiens
LWEEQLAAAK

SEQ ID NO: 427 - protein, 12 aa, Homo sapiens
ASTATAEQFFQK

SEQ ID NO: 428 - protein, 9 aa, Homo sapiens
AFLFQDTPR

SEQ ID NO: 429 - protein, 10 aa, Homo sapiens
TCLDEMNVVR

SEQ ID NO: 430 - protein, 10 aa, Homo sapiens
KGSPDDVEFK

SEQ ID NO: 431 - protein, 13 aa, Homo sapiens
DFLPVDTSNNNGR

SEQ ID NO: 432 - protein, 21 aa, Homo sapiens
SEHPESSLSSEEETAGVENVK

SEQ ID NO: 433 - protein, 18 aa, Homo sapiens
LAACLEGNCAEGLGTNYR

SEQ ID NO: 434 - protein, 19 aa, Homo sapiens
LAVTTHGLPCLAWASAQAK

SEQ ID NO: 435 - protein, 12 aa, Homo sapiens
VTVLDTNDNAPK

SEQ ID NO: 436 - protein, 8 aa, Homo sapiens
APIIAVTR

SEQ ID NO: 437 - protein, 14 aa, Homo sapiens
FPLEMQIYCFDADR

SEQ ID NO: 438 - protein, 11 aa, Homo sapiens
IISVELPGDAK

SEQ ID NO: 439 - protein, 15 aa, Homo sapiens
CSGSVSQPSVFFPTK

SEQ ID NO: 440 - protein, 8 aa, Homo sapiens
EEAPLGSR

SEQ ID NO: 441 - protein, 11 aa, Homo sapiens
FFIVACDPPQK

SEQ ID NO: 442 - protein, 21 aa, Homo sapiens
QHMDSDSSPSSSSTYCNQMMR

SEQ ID NO: 443 - protein, 9 aa, Homo sapiens
QEILAALEK

SEQ ID NO: 444 - protein, 9 aa, Homo sapiens
DGGFCEVCK

SEQ ID NO: 445 - protein, 9 aa, Homo sapiens
DGDFGSRLR

SEQ ID NO: 446 - protein, 17 aa, Homo sapiens
AIPVAQDLNAPSDWDSR

SEQ ID NO: 447 - protein, 24 aa, Homo sapiens
SAEFPDFYDSEEPVSTHQEAENEK

SEQ ID NO: 448 - protein, 16 aa, Homo sapiens
GEAGAPGEEDIQGPTK

SEQ ID NO: 449 - protein, 16 aa, Homo sapiens
SQREDEEEEEGENYQK

SEQ ID NO: 450 - protein, 13 aa, Homo sapiens
ASEEEPEYGEEIK

SEQ ID NO: 451 - protein, 13 aa, Homo sapiens
EDEEEEEGENYQK

SEQ ID NO: 452 - protein, 15 aa, Homo sapiens
KELENLAAMDLELQK

SEQ ID NO: 453 - protein, 16 aa, Homo sapiens
HLEEPGETQNAFLNER

SEQ ID NO: 454 - protein, 12 aa, Homo sapiens
ADQTVLTEDEKK

SEQ ID NO: 455 - protein, 12 aa, Homo sapiens
CIIEVLSNALSK

SEQ ID NO: 456 - protein, 26 aa, Homo sapiens
SAEFPDFYDSEEPVSTHQEAENEKDR

SEQ ID NO: 457 - protein, 17 aa, Homo sapiens
LLRDPADASEAHESSSR

SEQ ID NO: 458 - protein, 14 aa, Homo sapiens
DPADASEAHESSSR

SEQ ID NO: 459 - protein, 16 aa, Homo sapiens
LRDPADASEAHESSSR

SEQ ID NO: 460 - protein, 11 aa, Homo sapiens
LYTDDEDDIYK

SEQ ID NO: 461 - protein, 9 aa, Homo sapiens
TEAYLEAIR

SEQ ID NO: 462 - protein, 10 aa, Homo sapiens
FPKPGGSQDK

SEQ ID NO: 463 - protein, 24 aa, Homo sapiens
FQDDPDGLHQLDGTPLTAEDIVHK

SEQ ID NO: 464 - protein, 10 aa, Homo sapiens
GNKEDYDLSK

SEQ ID NO: 465 - protein, 19 aa, Homo sapiens
ELSAERPLNEQIAEAEEDK

SEQ ID NO: 466 - protein, 9 aa, Homo sapiens
LIDDYDSTK

SEQ ID NO: 467 - protein, 9 aa, Homo sapiens
IEFTSDQAR

SEQ ID NO: 468 - protein, 9 aa, Homo sapiens
VSLAEDDDR

SEQ ID NO: 469 - protein, 18 aa, Homo sapiens
NTGTEAPDYLATVDVDPK

SEQ ID NO: 470 - protein, 9 aa, Homo sapiens
RVSLATVDK

SEQ ID NO: 471 - protein, 10 aa, Homo sapiens
SDSEVAGYIR

SEQ ID NO: 472 - protein, 11 aa, Homo sapiens
IAQLPLTGSMS

SEQ ID NO: 473 - protein, 21 aa, Homo sapiens
ALVLIAFAQYLQQCPFEDHVK

SEQ ID NO: 474 - protein, 11 aa, Homo sapiens
EALQGVGDMGR

SEQ ID NO: 475 - protein, 8 aa, Homo sapiens
NNMDFSIR

SEQ ID NO: 476 - protein, 18 aa, Homo sapiens
AAAGGPGGAALGEAPPGR

SEQ ID NO: 477 - protein, 8 aa, Homo sapiens
AQLQEQVG

SEQ ID NO: 478 - protein, 16 aa, Homo sapiens
SYELPDGQVITIGNER

SEQ ID NO: 479 - protein, 9 aa, Homo sapiens
NVLVTLYER

SEQ ID NO: 480 - protein, 27 aa, Homo sapiens
KLSENTDFLAPGVSSFTDSNQQESITK

SEQ ID NO: 481 - protein, 9 aa, Homo sapiens
EEDIDENLL

SEQ ID NO: 482 - protein, 16 aa, Homo sapiens
SIPTCTDFEVIQFPLR

SEQ ID NO: 483 - protein, 8 aa, Homo sapiens
ETGVDLTK

SEQ ID NO: 484 - protein, 12 aa, Homo sapiens
GPVQGIINFEQK

SEQ ID NO: 485 - protein, 9 aa, Homo sapiens
YGNELAGSR

SEQ ID NO: 486 - protein, 9 aa, Homo sapiens
VCIAQGYQR

SEQ ID NO: 487 - protein, 12 aa, Homo sapiens
LDTLAQEVALLK

SEQ ID NO: 488 - protein, 10 aa, Homo sapiens
VLYLSAFTSK

SEQ ID NO: 489 - protein, 10 aa, Homo sapiens
NALALFVLPK

SEQ ID NO: 490 - protein, 14 aa, Homo sapiens
SSAEAQTPEDTPNK

SEQ ID NO: 491 - protein, 16 aa, Homo sapiens
SPYSYSTTAVVTNPKE

SEQ ID NO: 492 - protein, 23 aa, Homo sapiens
YTIAALLSPYSYSTTAVVTNPKE

SEQ ID NO: 493 - protein, 24 aa, Homo sapiens
RYTIAALLSPYSYSTTAVVTNPKE

SEQ ID NO: 494 - protein, 16 aa, Homo sapiens
VTNGAFTGEISPGMIK

SEQ ID NO: 495 - protein, 20 aa, Homo sapiens
IITHPNFNGNTLDNDIMLIK

SEQ ID NO: 496 - protein, 17 aa, Homo sapiens
LGEHNIDVLEGNEQFIN

SEQ ID NO: 497 - protein, 19 aa, Homo sapiens
LGEHNIDVLEGNEQFINAA

SEQ ID NO: 498 - protein, 9 aa, Homo sapiens
TASHGDLIR

SEQ ID NO: 499 - protein, 16 aa, Homo sapiens
LSIEDFTAYGGVFGNK

SEQ ID NO: 500 - protein, 20 aa, Homo sapiens
VGEEDEEAAEAEAEAEEAER

SEQ ID NO: 501 - protein, 31 aa, Homo sapiens
EAEGTEEGGEEEDDEEMDPQTIDSLIELSTK

SEQ ID NO: 502 - protein, 13 aa, Homo sapiens
PQTIDSLIELSTK

SEQ ID NO: 503 - protein, 21 aa, Homo sapiens
SYLSMVGSCCTSASPTVCFLK

SEQ ID NO: 504 - protein, 18 aa, Homo sapiens
SLGECCDVEDSTTCFNAK

SEQ ID NO: 505 - protein, 20 aa, Homo sapiens
SCESNSPFPVHPGTAECCTK

SEQ ID NO: 506 - protein, 23 aa, Homo sapiens
HQPQEFPTYVEPTNDEICEAFRK

SEQ ID NO: 507 - protein, 22 aa, Homo sapiens
HQPQEFPTYVEPTNDEICEAFR

SEQ ID NO: 508 - protein, 10 aa, Homo sapiens
GQYCYELDEK

SEQ ID NO: 509 - protein, 10 aa, Homo sapiens
VDTVDPPYPR

SEQ ID NO: 510 - protein, 17 aa, Homo sapiens
QDPPSVVVTSHQAPGEK

SEQ ID NO: 511 - protein, 9 aa, Homo sapiens
EDIFMETLK

SEQ ID NO: 512 - protein, 18 aa, Homo sapiens
KSDVGFLPPFPTLDPEEK

SEQ ID NO: 513 - protein, 14 aa, Homo sapiens
AESPEVCFNEESPK

SEQ ID NO: 514 - protein, 10 aa, Homo sapiens
ICAMEGLPQK

SEQ ID NO: 515 - protein, 20 aa, Homo sapiens
SLPAPWLSMAPVSWITPGLK

SEQ ID NO: 516 - protein, 37 aa, Homo sapiens
LHDNQNGWSGDSAPVELILSDETLPAPEFSPEPESGR

SEQ ID NO: 517 - protein, 9 aa, Homo sapiens
SDVVYTDWK

SEQ ID NO: 518 - protein, 8 aa, Homo sapiens
FYIASAFR

SEQ ID NO: 519 - protein, 9 aa, Homo sapiens
WFYIASAFR

SEQ ID NO: 520 - protein, 12 aa, Homo sapiens
TLMFGSYLDDEK

SEQ ID NO: 521 - protein, 17 aa, Homo sapiens
EQLGEFYEALDCLCIPR

SEQ ID NO: 522 - protein, 11 aa, Homo sapiens
ITLLSALVETR

SEQ ID NO: 523 - protein, 9 aa, Homo sapiens
EIGELYLPK

SEQ ID NO: 524 - protein, 11 aa, Homo sapiens
ATWSGAVLAGR

SEQ ID NO: 525 - protein, 11 aa, Homo sapiens
SASNMAIVDVK

SEQ ID NO: 526 - protein, 12 aa, Homo sapiens
NEDSLVFVQTDK

SEQ ID NO: 527 - protein, 11 aa, Homo sapiens
VGFYESDVMGR

SEQ ID NO: 528 - protein, 19 aa, Homo sapiens
VVAQGVGIPEDSIFTMADR

SEQ ID NO: 529 - protein, 11 aa, Homo sapiens
TVAACNLPIVR

SEQ ID NO: 530 - protein, 8 aa, Homo sapiens
ETLLQDFR

SEQ ID NO: 531 - protein, 10 aa, Homo sapiens
QHVAAVDPEK

SEQ ID NO: 532 - protein, 12 aa, Homo sapiens
EVPLNTIIFMGR

SEQ ID NO: 533 - protein, 16 aa, Homo sapiens
EQLQDMGLVDLFSPEK

SEQ ID NO: 534 - protein, 11 aa, Homo sapiens
DDLYVSDAFHK

SEQ ID NO: 535 - protein, 11 aa, Homo sapiens
ATEHLSTLSEK

SEQ ID NO: 536 - protein, 11 aa, Homo sapiens
DLATVYVDVLK

SEQ ID NO: 537 - protein, 16 aa, Homo sapiens
EQLGPVTQEFWDNLEK

SEQ ID NO: 538 - protein, 10 aa, Homo sapiens
SKEQLTPLIK

SEQ ID NO: 539 - protein, 10 aa, Homo sapiens
ALVQQMEQLR

SEQ ID NO: 540 - protein, 9 aa, Homo sapiens
LEEQAQQIR

SEQ ID NO: 541 - protein, 9 aa, Homo sapiens
EHSSLAFWK

SEQ ID NO: 542 - protein, 20 aa, Homo sapiens
TFYEPGEEITYSCKPGYVSR

SEQ ID NO: 543 - protein, 23 aa, Homo sapiens
CPFPSRPDNGFVNYPAKPTLYYK

SEQ ID NO: 544 - protein, 10 aa, Homo sapiens
NQEACELSNN

SEQ ID NO: 545 - protein, 18 aa, Homo sapiens
GSVYIGGAPDVATLTGGR

SEQ ID NO: 546 - protein, 17 aa, Homo sapiens
TSIYPFLDFMPSPQVVR

SEQ ID NO: 547 - protein, 9 aa, Homo sapiens
SSELKTACR

SEQ ID NO: 548 - protein, 12 aa, Homo sapiens
LSLEIEQLELQR

SEQ ID NO: 549 - protein, 12 aa, Homo sapiens
DVHEGQPLLNVK

SEQ ID NO: 550 - protein, 8 aa, Homo sapiens
KSLEELSK

SEQ ID NO: 551 - protein, 8 aa, Homo sapiens
VLCLAGFR

SEQ ID NO: 552 - protein, 21 aa, Homo sapiens
SGSAHEYSSSPDDAIFQSLAR

SEQ ID NO: 553 - protein, 13 aa, Homo sapiens
GAYPLSIEPIGVR

SEQ ID NO: 554 - protein, 14 aa, Homo sapiens
DIASGLIGPLIICK

SEQ ID NO: 555 - protein, 12 aa, Homo sapiens
PQALPEPMQESK

SEQ ID NO: 556 - protein, 21 aa, Homo sapiens
SGEATDGARPQALPEPMQESK

SEQ ID NO: 557 - protein, 18 aa, Homo sapiens
GPQAEGDSEGLSQGLVDR

SEQ ID NO: 558 - protein, 16 aa, Homo sapiens
EPQDTYHYLPFSLPHR

SEQ ID NO: 559 - protein, 9 aa, Homo sapiens
SALVLQYLR

SEQ ID NO: 560 - protein, 10 aa, Homo sapiens
SEAYNTFSER

SEQ ID NO: 561 - protein, 18 aa, Homo sapiens
LHEAFSPVSYQHDLALLR

SEQ ID NO: 562 - protein, 8 aa, Homo sapiens
SDFSNEER

SEQ ID NO: 563 - protein, 8 aa, Homo sapiens
EILNINQK

SEQ ID NO: 564 - protein, 10 aa, Homo sapiens
HAIILLTDGK

SEQ ID NO: 565 - protein, 9 aa, Homo sapiens
GYTQQLAFR

SEQ ID NO: 566 - protein, 16 aa, Homo sapiens
SNLDEDIIAEENIVSR

SEQ ID NO: 567 - protein, 11 aa, Homo sapiens
RHQQTVTIPPK

SEQ ID NO: 568 - protein, 8 aa, Homo sapiens
ISLPESLK

SEQ ID NO: 569 - protein, 12 aa, Homo sapiens
VSHSEDDCLAFK

SEQ ID NO: 570 - protein, 13 aa, Homo sapiens
AAVYHHFISDGVR

SEQ ID NO: 571 - protein, 15 aa, Homo sapiens
RIPIEDGSGEVVLSR

SEQ ID NO: 572 - protein, 10 aa, Homo sapiens
DFDFVPPVVR

SEQ ID NO: 573 - protein, 14 aa, Homo sapiens
IPIEDGSGEVVLSR

SEQ ID NO: 574 - protein, 16 aa, Homo sapiens
VELLHNPAFCSLATTK

SEQ ID NO: 575 - protein, 18 aa, Homo sapiens
AYYENSPQQVFSTEFEVK

SEQ ID NO: 576 - protein, 17 aa, Homo sapiens
LSINTHPSQKPLSITVR

SEQ ID NO: 577 - protein, 10 aa, Homo sapiens
FYHPEKEDGK

SEQ ID NO: 578 - protein, 17 aa, Homo sapiens
SEETKENEGFTVTAEGK

SEQ ID NO: 579 - protein, 8 aa, Homo sapiens
FYYIYNEK

SEQ ID NO: 580 - protein, 13 aa, Homo sapiens
SSLSVPYVIVPLK

SEQ ID NO: 581 - protein, 16 aa, Homo sapiens
SGIPIVTSPYQIHFTK

SEQ ID NO: 582 - protein, 8 aa, Homo sapiens
SAFAAFVK

SEQ ID NO: 583 - protein, 14 aa, Homo sapiens
KVFLDCCNYITELR

SEQ ID NO: 584 - protein, 19 aa, Homo sapiens
GLSSDFWGEKPNLSYIIGK

SEQ ID NO: 585 - protein, 12 aa, Homo sapiens
TIYTPGSTVLYR

SEQ ID NO: 586 - protein, 12 aa, Homo sapiens
EVVADSVWVDVK

SEQ ID NO: 587 - protein, 8 aa, Homo sapiens
GVFVLNKK

SEQ ID NO: 588 - protein, 10 aa, Homo sapiens
VTCTNAELVK

SEQ ID NO: 589 - protein, 8 aa, Homo sapiens
SGFSFGFK

SEQ ID NO: 590 - protein, 14 aa, Homo sapiens
LEQGENVFLQATDK

SEQ ID NO: 591 - protein, 11 aa, Homo sapiens
ASAGLLGAHAA

SEQ ID NO: 592 - protein, 7 aa, Homo sapiens
IPYSVVR

SEQ ID NO: 593 - protein, 20 aa, Homo sapiens
LIDQYGTHYLQSGSLGGEYR

SEQ ID NO: 594 - protein, 20 aa, Homo sapiens
VPANLENVGFEVQTAEDDLK

SEQ ID NO: 595 - protein, 15 aa, Homo sapiens
YYAGGCSPHYILNTR

SEQ ID NO: 596 - protein, 8 aa, Homo sapiens
FLQEQGHR

SEQ ID NO: 597 - protein, 11 aa, Homo sapiens
TSNFNAAISLK

SEQ ID NO: 598 - protein, 12 aa, Homo sapiens
AIEDYINEFSVR

SEQ ID NO: 599 - protein, 11 aa, Homo sapiens
LSPIYNLVPVK

SEQ ID NO: 600 - protein, 9 aa, Homo sapiens
VASYGVKPR

SEQ ID NO: 601 - protein, 9 aa, Homo sapiens
EYHFGQAVR

SEQ ID NO: 602 - protein, 11 aa, Homo sapiens
SCDIPVFMNAR

SEQ ID NO: 603 - protein, 23 aa, Homo sapiens
NTEILTGSWSDQTYPEGTQAIYK

SEQ ID NO: 604 - protein, 11 aa, Homo sapiens
INHGILYDEEK

SEQ ID NO: 605 - protein, 10 aa, Homo sapiens
TGDIVEFVCK

SEQ ID NO: 606 - protein, 10 aa, Homo sapiens
VFSLQWGEVK

SEQ ID NO: 607 - protein, 9 aa, Homo sapiens
VEVLAGDLR

SEQ ID NO: 608 - protein, 19 aa, Homo sapiens
FVSQTNGNLYIANVEASDK

SEQ ID NO: 609 - protein, 14 aa, Homo sapiens
VSPAAGSSPGKPPR

SEQ ID NO: 610 - protein, 12 aa, Homo sapiens
RALDFAVGEYNK

SEQ ID NO: 611 - protein, 9 aa, Homo sapiens
KDQEGGEEK

SEQ ID NO: 612 - protein, 14 aa, Homo sapiens
SGPGAYESGIMVSK

SEQ ID NO: 613 - protein, 12 aa, Homo sapiens
CFFQGDHGFDNK

SEQ ID NO: 614 - protein, 13 aa, Homo sapiens
AVVVHAGEDDLGR

SEQ ID NO: 615 - protein, 10 aa, Homo sapiens
VSYVGLVTVR

SEQ ID NO: 616 - protein, 13 aa, Homo sapiens
VNKDDEEFIESNK

SEQ ID NO: 617 - protein, 20 aa, Homo sapiens
KLISVDTEHSNIYLQNGPDR

SEQ ID NO: 618 - protein, 13 aa, Homo sapiens
RQSEDSTFYLGER

SEQ ID NO: 619 - protein, 21 aa, Homo sapiens
KAEEEHLGILGPQLHADVGDK

SEQ ID NO: 620 - protein, 11 aa, Homo sapiens
DCSLPYATESK

SEQ ID NO: 621 - protein, 7 aa, Homo sapiens
EQLMAEK

SEQ ID NO: 622 - protein, 7 aa, Homo sapiens
VDLVLEK

SEQ ID NO: 623 - protein, 11 aa, Homo sapiens
TVQLDDYLNGR

SEQ ID NO: 624 - protein, 9 aa, Homo sapiens
QDTTLNELQ

SEQ ID NO: 625 - protein, 20 aa, Homo sapiens
SPVGVQPILNEHTFCAGMSK

SEQ ID NO: 626 - protein, 16 aa, Homo sapiens
NVAEGETKPDPDVTER

SEQ ID NO: 627 - protein, 9 aa, Homo sapiens
DYFMPCPGR

SEQ ID NO: 628 - protein, 24 aa, Homo sapiens
EVGTPHGIILDSVDAAFICPGSSR

SEQ ID NO: 629 - protein, 10 aa, Homo sapiens
QGHNSVFLIK

SEQ ID NO: 630 - protein, 21 aa, Homo sapiens
VPGPAEGPAEPAAEASDEAER

SEQ ID NO: 631 - protein, 12 aa, Homo sapiens
TPLGDTTHTCPR

SEQ ID NO: 632 - protein, 18 aa, Homo sapiens
SDSGTYGCTATSNMGSYK

SEQ ID NO: 633 - protein, 10 aa, Homo sapiens
DGGELPDPDR

SEQ ID NO: 634 - protein, 8 aa, Homo sapiens
YDLSALVR

SEQ ID NO: 635 - protein, 16 aa, Homo sapiens
TELLPGDRDNLAIQTR

SEQ ID NO: 636 - protein, 12 aa, Homo sapiens
GPDVLTATVSGK

SEQ ID NO: 637 - protein, 10 aa, Homo sapiens
LDAQASFLPK

SEQ ID NO: 638 - protein, 19 aa, Homo sapiens
GSLVQASEANLQAAQDFVR

SEQ ID NO: 639 - protein, 11 aa, Homo sapiens
LSNENHGIAQR

SEQ ID NO: 640 - protein, 9 aa, Homo sapiens
VYQGEITER

SEQ ID NO: 641 - protein, 12 aa, Homo sapiens
AAISGENAGLVR

SEQ ID NO: 642 - protein, 13 aa, Homo sapiens
KAAISGENAGLVR

SEQ ID NO: 643 - protein, 10 aa, Homo sapiens
TEVNVLPGAK

SEQ ID NO: 644 - protein, 15 aa, Homo sapiens
AEDHFSVIDFNQNIR

SEQ ID NO: 645 - protein, 10 aa, Homo sapiens
AEAESLYQSK

SEQ ID NO: 646 - protein, 9 aa, Homo sapiens
QVVAGLNFR

SEQ ID NO: 647 - protein, 17 aa, Homo sapiens
IYPTVNCQPLGMISLMK

SEQ ID NO: 648 - protein, 9 aa, Homo sapiens
GPVGVQTFR

SEQ ID NO: 649 - protein, 8 aa, Homo sapiens
MSPHLTAL

SEQ ID NO: 650 - protein, 11 aa, Homo sapiens
AANEVSSADVK

SEQ ID NO: 651 - protein, 10 aa, Homo sapiens
LKDDEVAQLK

SEQ ID NO: 652 - protein, 7 aa, Homo sapiens
LVIAFGR

SEQ ID NO: 653 - protein, 12 aa, Homo sapiens
VVEQMCITQYER

SEQ ID NO: 654 - protein, 9 aa, Homo sapiens
LGVTANDVK

SEQ ID NO: 655 - protein, 9 aa, Homo sapiens
QESSADLLQ

SEQ ID NO: 656 - protein, 15 aa, Homo sapiens
LTIYNANIEDAGIYR

SEQ ID NO: 657 - protein, 9 aa, Homo sapiens
MGSISFDFR

SEQ ID NO: 658 - protein, 13 aa, Homo sapiens
EQGTQDITDASER

SEQ ID NO: 659 - protein, 10 aa, Homo sapiens
LSSAIIAAPR

SEQ ID NO: 660 - protein, 13 aa, Homo sapiens
VVVNFAPTIQEIK

SEQ ID NO: 661 - protein, 9 aa, Homo sapiens
VAQLPLSLK

SEQ ID NO: 662 - protein, 7 aa, Homo sapiens
LMNIVNK

SEQ ID NO: 663 - protein, 16 aa, Homo sapiens
YPDAVATWLNPDPSQK

SEQ ID NO: 664 - protein, 10 aa, Homo sapiens
IAQLPLTGSM

SEQ ID NO: 665 - protein, 12 aa, Homo sapiens
DTDTGALLFIGK

SEQ ID NO: 666 - protein, 9 aa, Homo sapiens
GEIQNILQK

SEQ ID NO: 667 - protein, 10 aa, Homo sapiens
YWGVASFLQK

SEQ ID NO: 668 - protein, 8 aa, Homo sapiens
TPENFPCK

SEQ ID NO: 669 - protein, 7 aa, Homo sapiens
YEFLNGR

SEQ ID NO: 670 - protein, 10 aa, Homo sapiens
EAQLPVIENK

SEQ ID NO: 671 - protein, 10 aa, Homo sapiens
LSSPAVITDK

SEQ ID NO: 672 - protein, 16 aa, Homo sapiens
TPENYPNAGLTMNYCR

SEQ ID NO: 673 - protein, 19 aa, Homo sapiens
NPDNDPQGPWCYTTDPEKR

SEQ ID NO: 674 - protein, 11 aa, Homo sapiens
EQQCVIMAENR

SEQ ID NO: 675 - protein, 10 aa, Homo sapiens
FVTWIEGVMR

SEQ ID NO: 676 - protein, 15 aa, Homo sapiens
AEEDDSLANSSDLLK

SEQ ID NO: 677 - protein, 10 aa, Homo sapiens
PDAPAAQLAR

SEQ ID NO: 678 - protein, 10 aa, Homo sapiens
ALAHLLEAER

SEQ ID NO: 679 - protein, 13 aa, Homo sapiens
TEDTIVFLPQTDK

SEQ ID NO: 680 - protein, 13 aa, Homo sapiens
TMLLQPAGSLGSY

SEQ ID NO: 681 - protein, 9 aa, Homo sapiens
ALLYSQGSK

SEQ ID NO: 682 - protein, 9 aa, Homo sapiens
TMLLQPAGS

SEQ ID NO: 683 - protein, 14 aa, Homo sapiens
EAQVSVQPNFQQDK

SEQ ID NO: 684 - protein, 12 aa, Homo sapiens
TMLLQPAGSLGS

SEQ ID NO: 685 - protein, 7 aa, Homo sapiens
GPGEDFR

SEQ ID NO: 686 - protein, 9 aa, Homo sapiens
AFLFQDTPR

SEQ ID NO: 687 - protein, 13 aa, Homo sapiens
VPPTLEVTQQPVR

SEQ ID NO: 688 - protein, 10 aa, Homo sapiens
ELLESYIDGR

SEQ ID NO: 689 - protein, 15 aa, Homo sapiens
SPQELLCGASLISDR

SEQ ID NO: 690 - protein, 7 aa, Homo sapiens
VIDQFGE

SEQ ID NO: 691 - protein, 11 aa, Homo sapiens
VSGGVSEMVFK

SEQ ID NO: 692 - protein, 7 aa, Homo sapiens
LVGYLDR

SEQ ID NO: 693 - protein, 16 aa, Homo sapiens
GEAGAPGEEDIQGPTK

SEQ ID NO: 694 - protein, 15 aa, Homo sapiens
KELENLAAMDLELQK

SEQ ID NO: 695 - protein, 16 aa, Homo sapiens
HLEEPGETQNAFLNER

SEQ ID NO: 696 - protein, 10 aa, Homo sapiens
IESQTQEEVR

SEQ ID NO: 697 - protein, 13 aa, Homo sapiens
GDQGGESSLSVSK

SEQ ID NO: 698 - protein, 15 aa, Homo sapiens
LDLQEINNWVQAQMK

SEQ ID NO: 699 - protein, 13 aa, Homo sapiens
LEAGDHPVELLAR

SEQ ID NO: 700 - protein, 19 aa, Homo sapiens
TVSEALLMEPTDDGNTTPR

SEQ ID NO: 701 - protein, 11 aa, Homo sapiens
VLTHSELAPLR

SEQ ID NO: 702 - protein, 10 aa, Homo sapiens
TGLEAISNHK

SEQ ID NO: 703 - protein, 9 aa, Homo sapiens
SSSQELGLK

SEQ ID NO: 704 - protein, 19 aa, Homo sapiens
VHENENIGTTEPGEHQEAK

SEQ ID NO: 705 - protein, 14 aa, Homo sapiens
GDGPVQGIINFEQK

SEQ ID NO: 706 - protein, 8 aa, Homo sapiens
LTFVHGGK

SEQ ID NO: 707 - protein, 12 aa, Homo sapiens
SYSTTAVVTNPK

SEQ ID NO: 708 - protein, 13 aa, Homo sapiens
IIYGGSVTGATCK

SEQ ID NO: 709 - protein, 15 aa, Homo sapiens
VVLAYEPVWAIGTGK

SEQ ID NO: 710 - protein, 10 aa, Homo sapiens
THLPEVFLSK

SEQ ID NO: 711 - protein, 11 aa, Homo sapiens
HLSLLTTLSNR

SEQ ID NO: 712 - protein, 8 aa, Homo sapiens
ELSSFIDK

SEQ ID NO: 713 - protein, 11 aa, Homo sapiens
RTHLPEVFLSK

SEQ ID NO: 714 - protein, 13 aa, Homo sapiens
EDFTSLSLVLYSR

SEQ ID NO: 715 - protein, 15 aa, Homo sapiens
KFPSGTFEQVSQLVK

SEQ ID NO: 716 - protein, 15 aa, Homo sapiens
SIAQYWLGCPAPGHL

SEQ ID NO: 717 - protein, 15 aa, Homo sapiens
DVWGIEGPIDAAFTR

SEQ ID NO: 718 - protein, 12 aa, Homo sapiens
AYLEEECPATLR

SEQ ID NO: 719 - protein, 10 aa, Homo sapiens
WEAEPVYVQR

SEQ ID NO: 720 - protein, 10 aa, Homo sapiens
AGEVQEPELR

SEQ ID NO: 721 - protein, 9 aa, Homo sapiens
EDIFMETLK

SEQ ID NO: 722 - protein, 12 aa, Homo sapiens
ALYLQYTDETFR

SEQ ID NO: 723 - protein, 21 aa, Homo sapiens
AEDEENEKETAVSTEDDSHHK

SEQ ID NO: 724 - protein, 12 aa, Homo sapiens
RLPLEYSYGEYR

SEQ ID NO: 725 - protein, 7 aa, Homo sapiens
IFTVNHK

SEQ ID NO: 726 - protein, 15 aa, Homo sapiens
SSSNEEVMFLTVQVK

SEQ ID NO: 727 - protein, 8 aa, Homo sapiens
SHAAEPAR

SEQ ID NO: 728 - protein, 15 aa, Homo sapiens
NLQNNAEWVYQGAIR

SEQ ID NO: 729 - protein, 20 aa, Homo sapiens
SITCIHGVWTQLPQCVAIDK

SEQ ID NO: 730 - protein, 24 aa, Homo sapiens
QPDGTPGGSGAAVAPAAGQGSHSR

SEQ ID NO: 731 - protein, 10 aa, Homo sapiens
DPNPIIDGRK

SEQ ID NO: 732 - protein, 8 aa, Homo sapiens
DFHINLFR

SEQ ID NO: 733 - protein, 10 aa, Homo sapiens
EYESYSDFER

SEQ ID NO: 734 - protein, 9 aa, Homo sapiens
FMQAVTGWK

SEQ ID NO: 735 - protein, 13 aa, Homo sapiens
TAFQEALDAAGDK

SEQ ID NO: 736 - protein, 7 aa, Homo sapiens
QTQEILR

SEQ ID NO: 737 - protein, 11 aa, Homo sapiens
DIPMNPMCIYR

SEQ ID NO: 738 - protein, 11 aa, Homo sapiens
CNTPAMGLQKK

SEQ ID NO: 739 - protein, 7 aa, Homo sapiens
RVWELSK

SEQ ID NO: 740 - protein, 11 aa, Homo sapiens
LKEFGNTLEDK

SEQ ID NO: 741 - protein, 10 aa, Homo sapiens
VTSIQDWVQK

SEQ ID NO: 742 - protein, 8 aa, Homo sapiens
LVVLPFPK

SEQ ID NO: 743 - protein, 9 aa, Homo sapiens
ISLPESLKR

SEQ ID NO: 744 - protein, 14 aa, Homo sapiens
TPKSPPDPPLDIPR

SEQ ID NO: 745 - protein, 9 aa, Homo sapiens
TNFDVYSQK

SEQ ID NO: 746 - protein, 8 aa, Homo sapiens
IEDGFSLK

SEQ ID NO: 747 - protein, 9 aa, Homo sapiens
VVEESELAR

SEQ ID NO: 748 - protein, 9 aa, Homo sapiens
KMKERPDLK

SEQ ID NO: 749 - protein, 13 aa, Homo sapiens
LRHFGSSLSLPDR

SEQ ID NO: 750 - protein, 12 aa, Homo sapiens
KGNVATEISTER

SEQ ID NO: 751 - protein, 6 aa, Homo sapiens
QADIIK

SEQ ID NO: 752 - protein, 8 aa, Homo sapiens
WELCDIPR

SEQ ID NO: 753 - protein, 9 aa, Homo sapiens
TGLQEVEVK

SEQ ID NO: 754 - protein, 9 aa, Homo sapiens
ALLNHGAVR

SEQ ID NO: 755 - protein, 6 aa, Homo sapiens
DSLEFR

SEQ ID NO: 756 - protein, 9 aa, Homo sapiens
DVCDPGNTK

SEQ ID NO: 757 - protein, 5 aa, Homo sapiens
QASVR

SEQ ID NO: 758 - protein, 10 aa, Homo sapiens
KPNLQVFLGK

SEQ ID NO: 759 - protein, 7 aa, Homo sapiens
VVPEGIR

SEQ ID NO: 760 - protein, 9 aa, Homo sapiens
CLAPLEGAR

SEQ ID NO: 761 - protein, 9 aa, Homo sapiens
EKLLELIRR

SEQ ID NO: 762 - protein, 8 aa, Homo sapiens
LCDNLSTK

SEQ ID NO: 763 - protein, 13 aa, Homo sapiens
SLPSEASEQYLTK

SEQ ID NO: 764 - protein, 6 aa, Homo sapiens
LLQDMR

SEQ ID NO: 765 - protein, 8 aa, Homo sapiens
QRVGEISK

SEQ ID NO: 766 - protein, 13 aa, Homo sapiens
EAGSAVEAEELVK

SEQ ID NO: 767 - protein, 7 aa, Homo sapiens
EALQMLR

SEQ ID NO: 768 - protein, 5 aa, Homo sapiens
VNFLR

SEQ ID NO: 769 - protein, 7 aa, Homo sapiens
LWWLDLK

SEQ ID NO: 770 - protein, 9 aa, Homo sapiens
GNPAVSYVR

SEQ ID NO: 771 - protein, 7 aa, Homo sapiens
LINDYVK

SEQ ID NO: 772 - protein, 5 aa, Homo sapiens
LNELR

SEQ ID NO: 773 - protein, 8 aa, Homo sapiens
CLVNLIEK

SEQ ID NO: 774 - protein, 9 aa, Homo sapiens
AKPALEDLR

SEQ ID NO: 775 - protein, 8 aa, Homo sapiens
KLINDYVK

SEQ ID NO: 776 - protein, 9 aa, Homo sapiens
ITQVLHFTK

SEQ ID NO: 777 - protein, 19 aa, Homo sapiens
EVCCVEEVLNLVEIGNSCR

SEQ ID NO: 778 - protein, 11 aa, Homo sapiens
VCSQYAAYGEK

SEQ ID NO: 779 - protein, 10 aa, Homo sapiens
VEHSDLSFSK

SEQ ID NO: 780 - protein, 9 aa, Homo sapiens
GLQEAAEER

SEQ ID NO: 781 - protein, 11 aa, Homo sapiens
TYGLPCHCPFK

SEQ ID NO: 782 - protein, 8 aa, Homo sapiens
DKCEPLEK

SEQ ID NO: 783 - protein, 10 aa, Homo sapiens
LETPDFQLFK

SEQ ID NO: 784 - protein, 13 aa, Homo sapiens
FSSSSGYGGGSSR

SEQ ID NO: 785 - protein, 18 aa, Homo sapiens
LREQLGPVTQEFWDNLEK

SEQ ID NO: 786 - protein, 12 aa, Homo sapiens
VLCDDVICDETK

SEQ ID NO: 787 - protein, 11 aa, Homo sapiens
GCSFLPDPYQK

SEQ ID NO: 788 - protein, 6 aa, Homo sapiens
CEPLEK

SEQ ID NO: 789 - protein, 8 aa, Homo sapiens
RLWWLDLK

SEQ ID NO: 790 - protein, 10 aa, Homo sapiens
GGYVSFNYLK

SEQ ID NO: 791 - protein, 13 aa, Homo sapiens
GQAGDASLMELEK

SEQ ID NO: 792 - protein, 10 aa, Homo sapiens
GYSFTTTAER

SEQ ID NO: 793 - protein, 17 aa, Homo sapiens
SDVGFLPPFPTLDPEEK

SEQ ID NO: 794 - protein, 11 aa, Homo sapiens
FTDSENVCQER

SEQ ID NO: 795 - protein, 10 aa, Homo sapiens
MLAFDVNDEK

SEQ ID NO: 796 - protein, 13 aa, Homo sapiens
GGQEHFAHLLILR

SEQ ID NO: 797 - protein, 15 aa, Homo sapiens
NWGLSVYADKPETTK

SEQ ID NO: 798 - protein, 15 aa, Homo sapiens
NWGLSFYADKPETTK

SEQ ID NO: 799 - protein, 9 aa, Homo sapiens
SDVMYTDWK

SEQ ID NO: 800 - protein, 11 aa, Homo sapiens
SFYADKPETTK

SEQ ID NO: 801 - protein, 11 aa, Homo sapiens
SFYADKPETTK

SEQ ID NO: 802 - protein, 10 aa, Homo sapiens
EALDCLCIPR

SEQ ID NO: 803 - protein, 14 aa, Homo sapiens
MEEVEAMLLPETLK

SEQ ID NO: 804 - protein, 14 aa, Homo sapiens
WEMPFDPQDTHQSR

SEQ ID NO: 805 - protein, 11 aa, Homo sapiens
LCQDLGPGAFR

SEQ ID NO: 806 - protein, 24 aa, Homo sapiens
AQLVPLPPSTYVEFTVSGTDCVAK

SEQ ID NO: 807 - protein, 14 aa, Homo sapiens
EHAVEGDCDFQLLK

SEQ ID NO: 808 - protein, 17 aa, Homo sapiens
QLKEHAVEGDCDFQLLK

SEQ ID NO: 809 - protein, 12 aa, Homo sapiens
VTAAPQSVCALR

SEQ ID NO: 810 - protein, 11 aa, Homo sapiens
NALFCLESAWK

SEQ ID NO: 811 - protein, 17 aa, Homo sapiens
AFQPFFVELTMPYSVIR

SEQ ID NO: 812 - protein, 12 aa, Homo sapiens
EQAPHCICANGR

SEQ ID NO: 813 - protein, 14 aa, Homo sapiens
TEHPFTVEEFVLPK

SEQ ID NO: 814 - protein, 14 aa, Homo sapiens
TEVSSNHVLIYLDK

SEQ ID NO: 815 - protein, 9 aa, Homo sapiens
ATVLNYLPK

SEQ ID NO: 816 - protein, 16 aa, Homo sapiens
ALLAYAFALAGNQDKR

SEQ ID NO: 817 - protein, 16 aa, Homo sapiens
QFSFPLSSEPFQGSYK

SEQ ID NO: 818 - protein, 11 aa, Homo sapiens
LPPNVVEESAR

SEQ ID NO: 819 - protein, 11 aa, Homo sapiens
AIGYLNTGYQR

SEQ ID NO: 820 - protein, 23 aa, Homo sapiens
QQNAQGGFSSTQDTVVALHALSK

SEQ ID NO: 821 - protein, 19 aa, Homo sapiens
VDLSFSPSQSLPASHAHLR

SEQ ID NO: 822 - protein, 10 aa, Homo sapiens
QTVSWAVTPK

SEQ ID NO: 823 - protein, 12 aa, Homo sapiens
HYDGSYSTFGER

SEQ ID NO: 824 - protein, 17 aa, Homo sapiens
KDTVIKPLLVEPEGLEK

SEQ ID NO: 825 - protein, 9 aa, Homo sapiens
SLNEEAVKK

SEQ ID NO: 826 - protein, 13 aa, Homo sapiens
LVHVEEPHTETVR

SEQ ID NO: 827 - protein, 18 aa, Homo sapiens
LLLQQVSLPELPGEYSMK

SEQ ID NO: 828 - protein, 13 aa, Homo sapiens
NQGNTWLTAFVLK

SEQ ID NO: 829 - protein, 13 aa, Homo sapiens
QFSFPLSSEPFQG

SEQ ID NO: 830 - protein, 16 aa, Homo sapiens
QFSFPLSSEPFQGSYK

SEQ ID NO: 831 - protein, 16 aa, Homo sapiens
EQQDSPGNKDFLQSLK

SEQ ID NO: 832 - protein, 14 aa, Homo sapiens
WYNLAIGSTCPWLK

SEQ ID NO: 833 - protein, 13 aa, Homo sapiens
KGVCEETSGAYEK

SEQ ID NO: 834 - protein, 8 aa, Homo sapiens
VEAMLNDR

SEQ ID NO: 835 - protein, 14 aa, Homo sapiens
GLTTRPGSGLTNIK

SEQ ID NO: 836 - protein, 8 aa, Homo sapiens
TEEISEVK

SEQ ID NO: 837 - protein, 12 aa, Homo sapiens
VESLEQEAANER

SEQ ID NO: 838 - protein, 10 aa, Homo sapiens
QQLVETHMAR

SEQ ID NO: 839 - protein, 13 aa, Homo sapiens
AALEGFLAALQAD

SEQ ID NO: 840 - protein, 12 aa, Homo sapiens
GSTEQDAASPEK

SEQ ID NO: 841 - protein, 9 aa, Homo sapiens
VIALINDQR

SEQ ID NO: 842 - protein, 17 aa, Homo sapiens
QPFVQGLALYTPVVLPR

SEQ ID NO: 843 - protein, 12 aa, Homo sapiens
ALQDQLVLVAAK

SEQ ID NO: 844 - protein, 15 aa, Homo sapiens
VEGLTFQQNSLNWMK

SEQ ID NO: 845 - protein, 13 aa, Homo sapiens
FATTFYQHLADSK

SEQ ID NO: 846 - protein, 10 aa, Homo sapiens
FRIEDGFSLK

SEQ ID NO: 847 - protein, 14 aa, Homo sapiens
LGPVTQEFWDNLEK

SEQ ID NO: 848 - protein, 9 aa, Homo sapiens
LSPLGEEMR

SEQ ID NO: 849 - protein, 11 aa, Homo sapiens
LEALKENGGAR

SEQ ID NO: 850 - protein, 12 aa, Homo sapiens
KAGTELVNFLSY

SEQ ID NO: 851 - protein, 18 aa, Homo sapiens
CVESLVSQYFQTVTDYGK

SEQ ID NO: 852 - protein, 17 aa, Homo sapiens
LGPHAGDVEGHLSFLEK

SEQ ID NO: 853 - protein, 14 aa, Homo sapiens
SELTQQLNALFQDK

SEQ ID NO: 854 - protein, 9 aa, Homo sapiens
IPTTFENGR

SEQ ID NO: 855 - protein, 9 aa, Homo sapiens
LAVYQAGAR

SEQ ID NO: 856 - protein, 11 aa, Homo sapiens
AKLEEQAQQIR

SEQ ID NO: 857 - protein, 15 aa, Homo sapiens
AATVGSLAGQPLQER

SEQ ID NO: 858 - protein, 23 aa, Homo sapiens
WVQTLSEQVQEELLSSQVTQELR

SEQ ID NO: 859 - protein, 8 aa, Homo sapiens
ELQAAQAR

SEQ ID NO: 860 - protein, 10 aa, Homo sapiens
SLAGQPLQER

SEQ ID NO: 861 - protein, 10 aa, Homo sapiens
KATVVYQGER

SEQ ID NO: 862 - protein, 14 aa, Homo sapiens
TFYEPGEEITYSCK

SEQ ID NO: 863 - protein, 19 aa, Homo sapiens
KCSYTEDAQCIDGTIEVPK

SEQ ID NO: 864 - protein, 10 aa, Homo sapiens
HGQPVSFLLK

SEQ ID NO: 865 - protein, 12 aa, Homo sapiens
DIEHLTSLDFFR

SEQ ID NO: 866 - protein, 15 aa, Homo sapiens
RGSIQVDGEELVSGR

SEQ ID NO: 867 - protein, 10 aa, Homo sapiens
AGILENGAVR

SEQ ID NO: 868 - protein, 9 aa, Homo sapiens
TDASDVKPC

SEQ ID NO: 869 - protein, 12 aa, Homo sapiens
PFAGILENGAVR

SEQ ID NO: 870 - protein, 12 aa, Homo sapiens
LLYYTEFTPTEK

SEQ ID NO: 871 - protein, 7 aa, Homo sapiens
LSCMAIR

SEQ ID NO: 872 - protein, 14 aa, Homo sapiens
YAFSFSGAQEACAR

SEQ ID NO: 873 - protein, 14 aa, Homo sapiens
EACYGDMDGFPGVR

SEQ ID NO: 874 - protein, 12 aa, Homo sapiens
ELEAPSEDNSGR

SEQ ID NO: 875 - protein, 8 aa, Homo sapiens
ATEDVLVK

SEQ ID NO: 876 - protein, 9 aa, Homo sapiens
LICSELNGR

SEQ ID NO: 877 - protein, 12 aa, Homo sapiens
YGFIEGHVVIPR

SEQ ID NO: 878 - protein, 8 aa, Homo sapiens
AGLQVYNK

SEQ ID NO: 879 - protein, 12 aa, Homo sapiens
LRENELTYYCCK

SEQ ID NO: 880 - protein, 11 aa, Homo sapiens
GDLYFANVEEK

SEQ ID NO: 881 - protein, 19 aa, Homo sapiens
EAEEETTNDNGVLVLEPAR

SEQ ID NO: 882 - protein, 23 aa, Homo sapiens
SGAGTEDSACIPWAYYSTVDQVK

SEQ ID NO: 883 - protein, 19 aa, Homo sapiens
LISVDTEHSNIYLQNGPDR

SEQ ID NO: 884 - protein, 11 aa, Homo sapiens
DIFTGLIGPMK

SEQ ID NO: 885 - protein, 11 aa, Homo sapiens
VTIDSSYDIAK

SEQ ID NO: 886 - protein, 8 aa, Homo sapiens
FPLTNAIK

SEQ ID NO: 887 - protein, 24 aa, Homo sapiens
GEQEHSQQKEEEEEMAVVPQGLFR

SEQ ID NO: 888 - protein, 12 aa, Homo sapiens
ASSIIDELFQDR

SEQ ID NO: 889 - protein, 9 aa, Homo sapiens
IIDELFQDR

SEQ ID NO: 890 - protein, 17 aa, Homo sapiens
LFDSDPITVTVPVEVSR

SEQ ID NO: 891 - protein, 18 aa, Homo sapiens
DQTVSDNELQEMSNQGSK

SEQ ID NO: 892 - protein, 16 aa, Homo sapiens
VAVVQYSGTGQQRPER

SEQ ID NO: 893 - protein, 9 aa, Homo sapiens
FQSVFTVTR

SEQ ID NO: 894 - protein, 9 aa, Homo sapiens
VLNYVDWIK

SEQ ID NO: 895 - protein, 11 aa, Homo sapiens
TNFDNDIALVR

SEQ ID NO: 896 - protein, 15 aa, Homo sapiens
GDSGGAFAVQDPNDK

SEQ ID NO: 897 - protein, 11 aa, Homo sapiens
VEKPTADAEAY

SEQ ID NO: 898 - protein, 11 aa, Homo sapiens
GESGGAVFLER

SEQ ID NO: 899 - protein, 11 aa, Homo sapiens
QPVPGQQMTLK

SEQ ID NO: 900 - protein, 16 aa, Homo sapiens
EYVLPSFEVIVEPTEK

SEQ ID NO: 901 - protein, 8 aa, Homo sapiens
KLVLSSEK

SEQ ID NO: 902 - protein, 20 aa, Homo sapiens
GQDLVVLPLSITTDFIPSFR

SEQ ID NO: 903 - protein, 15 aa, Homo sapiens
TVMVNIENPEGIPVK

SEQ ID NO: 904 - protein, 14 aa, Homo sapiens
LVAYYTLIGASGQR

SEQ ID NO: 905 - protein, 12 aa, Homo sapiens
ENEGFTVTAEGK

SEQ ID NO: 906 - protein, 11 aa, Homo sapiens
ADIGCTPGSGK

SEQ ID NO: 907 - protein, 23 aa, Homo sapiens
QKPDGVFQEDAPVIHQEMIGGLR

SEQ ID NO: 908 - protein, 10 aa, Homo sapiens
KGYTQQLAFR

SEQ ID NO: 909 - protein, 11 aa, Homo sapiens
SITTDFIPSFR

SEQ ID NO: 910 - protein, 10 aa, Homo sapiens
CAEENCFIQK

SEQ ID NO: 911 - protein, 20 aa, Homo sapiens
EDIPPADLSDQVPDTESETR

SEQ ID NO: 912 - protein, 13 aa, Homo sapiens
VYAYYNLEESCTR

SEQ ID NO: 913 - protein, 8 aa, Homo sapiens
TYNVLDMK

SEQ ID NO: 914 - protein, 9 aa, Homo sapiens
GLQDEDGYR

SEQ ID NO: 915 - protein, 8 aa, Homo sapiens
ANSFLGEK

SEQ ID NO: 916 - protein, 15 aa, Homo sapiens
DFALLSLQVPLKDAK

SEQ ID NO: 917 - protein, 10 aa, Homo sapiens
SFFPENWLWR

SEQ ID NO: 918 - protein, 26 aa, Homo sapiens
VTASDPLDTLGSEGALSPGGVASLLR

SEQ ID NO: 919 - protein, 19 aa, Homo sapiens
ALEILQEEDLIDEDDIPVR

SEQ ID NO: 920 - protein, 13 aa, Homo sapiens
TDAPDLPEENQAR

SEQ ID NO: 921 - protein, 9 aa, Homo sapiens
GNLCVNLMR

SEQ ID NO: 922 - protein, 20 aa, Homo sapiens
LVFQQFDLEPSEGCFYDYVK

SEQ ID NO: 923 - protein, 18 aa, Homo sapiens
TLDEFTIIQNLQPQYQFR

SEQ ID NO: 924 - protein, 14 aa, Homo sapiens
LPVANPQACENWLR

SEQ ID NO: 925 - protein, 12 aa, Homo sapiens
WILTAAHTLYPK

SEQ ID NO: 926 - protein, 14 aa, Homo sapiens
EPFLSCCQFAESLR

SEQ ID NO: 927 - protein, 12 aa, Homo sapiens
KADGSYAAWLSR

SEQ ID NO: 928 - protein, 25 aa, Homo sapiens
STQDTVIALDALSAYWIASHTTEER

SEQ ID NO: 929 - protein, 20 aa, Homo sapiens
LLSQQQADGSFQDLSPVIHR

SEQ ID NO: 930 - protein, 17 aa, Homo sapiens
GHLFLQTDQPIYNPGQR

SEQ ID NO: 931 - protein, 14 aa, Homo sapiens
LGALDTALYAAGSK

SEQ ID NO: 932 - protein, 18 aa, Homo sapiens
AVGSGATFSHYYYMILSR

SEQ ID NO: 933 - protein, 14 aa, Homo sapiens
YIYGKPVQGVAYVR

SEQ ID NO: 934 - protein, 23 aa, Homo sapiens
YVSHFETEGPHVLLYFDSVPTSR

SEQ ID NO: 935 - protein, 10 aa, Homo sapiens
VGDTLNLNLR

SEQ ID NO: 936 - protein, 12 aa, Homo sapiens
IDTQDIEASHYR

SEQ ID NO: 937 - protein, 12 aa, Homo sapiens
FQNSAILTIQPK

SEQ ID NO: 938 - protein, 15 aa, Homo sapiens
ELSHLPSLYDYSAYR

SEQ ID NO: 939 - protein, 17 aa, Homo sapiens
GQSISVTSIRPCAAETQ

SEQ ID NO: 940 - protein, 11 aa, Homo sapiens
SYTSHTNEIHK

SEQ ID NO: 941 - protein, 15 aa, Homo sapiens
DGFVQDEGTMFPVGK

SEQ ID NO: 942 - protein, 18 aa, Homo sapiens
ENPAVIDFELAPIVDLVR

SEQ ID NO: 943 - protein, 11 aa, Homo sapiens
DRDGNTLTYYR

SEQ ID NO: 944 - protein, 11 aa, Homo sapiens
SIEVFGQFNGK

SEQ ID NO: 945 - protein, 18 aa, Homo sapiens
YGQTIRPICLPCTEGTTR

SEQ ID NO: 946 - protein, 11 aa, Homo sapiens
YGLVTYATYPK

SEQ ID NO: 947 - protein, 11 aa, Homo sapiens
LPPTTTCQQQK

SEQ ID NO: 948 - protein, 10 aa, Homo sapiens
ALFVSEEEKK

SEQ ID NO: 949 - protein, 18 aa, Homo sapiens
AVYTCNEGYQLLGEINYR

SEQ ID NO: 950 - protein, 9 aa, Homo sapiens
EFDHNSNIR

SEQ ID NO: 951 - protein, 14 aa, Homo sapiens
SIDVACHPGYALPK

SEQ ID NO: 952 - protein, 10 aa, Homo sapiens
WQSIPLCVEK

SEQ ID NO: 953 - protein, 18 aa, Homo sapiens
ECDTDGWTNDIPICEVVK

SEQ ID NO: 954 - protein, 10 aa, Homo sapiens
SLGNVIMVCR

SEQ ID NO: 955 - protein, 13 aa, Homo sapiens
ITCTEEGWSPTPK

SEQ ID NO: 956 - protein, 10 aa, Homo sapiens
TGESAEFVCK

SEQ ID NO: 957 - protein, 8 aa, Homo sapiens
IVIEYVDR

SEQ ID NO: 958 - protein, 16 aa, Homo sapiens
LSLEDSGMYQCVAENK

SEQ ID NO: 959 - protein, 18 aa, Homo sapiens
VIASNILGTGEPSGPSSK

SEQ ID NO: 960 - protein, 17 aa, Homo sapiens
TTGPGGDGIPAEVHIVR

SEQ ID NO: 961 - protein, 19 aa, Homo sapiens
TDGAAPNVAPSDVGGGGGR

SEQ ID NO: 962 - protein, 19 aa, Homo sapiens
VLEPMPSTAEISTSGAVLK

SEQ ID NO: 963 - protein, 7 aa, Homo sapiens
MSAQLAA

SEQ ID NO: 964 - protein, 14 aa, Homo sapiens
SAVEEMEAEEAAAK

SEQ ID NO: 965 - protein, 12 aa, Homo sapiens
EVEELMEDTQHK

SEQ ID NO: 966 - protein, 13 aa, Homo sapiens
YGPFGPEMTNPLR

SEQ ID NO: 967 - protein, 13 aa, Homo sapiens
TLQPGDGEASTPR

SEQ ID NO: 968 - protein, 13 aa, Homo sapiens
NVALVSGDTENAK

SEQ ID NO: 969 - protein, 10 aa, Homo sapiens
LNCEDIDECR

SEQ ID NO: 970 - protein, 11 aa, Homo sapiens
VTEIWQEVMQR

SEQ ID NO: 971 - protein, 8 aa, Homo sapiens
VTGVVLFR

SEQ ID NO: 972 - protein, 14 aa, Homo sapiens
FAVLQENVAWGNGR

SEQ ID NO: 973 - protein, 19 aa, Homo sapiens
MHSMNGFMYGNQPGLTMCK

SEQ ID NO: 974 - protein, 15 aa, Homo sapiens
GVYSSDVFDIFPGTY

SEQ ID NO: 975 - protein, 9 aa, Homo sapiens
VGPEADKYR

SEQ ID NO: 976 - protein, 14 aa, Homo sapiens
MGPTELLIEMEDWK

SEQ ID NO: 977 - protein, 10 aa, Homo sapiens
STTPDITGYR

SEQ ID NO: 978 - protein, 23 aa, Homo sapiens
GVVPLAGTNGETTTQGLDGLSER

SEQ ID NO: 979 - protein, 16 aa, Homo sapiens
DPDQTDGLGLSYLSSH

SEQ ID NO: 980 - protein, 13 aa, Homo sapiens
VHVSEEGTEPEAM

SEQ ID NO: 981 - protein, 17 aa, Homo sapiens
QTQVSVLPEGGETPLFK

SEQ ID NO: 982 - protein, 8 aa, Homo sapiens
TGAQELLR

SEQ ID NO: 983 - protein, 13 aa, Homo sapiens
AGALNSNDAFVLK

SEQ ID NO: 984 - protein, 9 aa, Homo sapiens
TGDAYVILK

SEQ ID NO: 985 - protein, 11 aa, Homo sapiens
HVVPNEVVVQR

SEQ ID NO: 986 - protein, 11 aa, Homo sapiens
AGKEPGLQIWR

SEQ ID NO: 987 - protein, 11 aa, Homo sapiens
MMAVAADTLQR

SEQ ID NO: 988 - protein, 16 aa, Homo sapiens
SGAQATWTELPWPHEK

SEQ ID NO: 989 - protein, 11 aa, Homo sapiens
NFPSPVDAAFR

SEQ ID NO: 990 - protein, 9 aa, Homo sapiens
VDGALCMEK

SEQ ID NO: 991 - protein, 9 aa, Homo sapiens
SVNDLYIQK

SEQ ID NO: 992 - protein, 9 aa, Homo sapiens
NYNLVESLK

SEQ ID NO: 993 - protein, 9 aa, Homo sapiens
QFPILLDFK

SEQ ID NO: 994 - protein, 9 aa, Homo sapiens
TSCLLFMGR

SEQ ID NO: 995 - protein, 9 aa, Homo sapiens
DGYLFQLLR

SEQ ID NO: 996 - protein, 16 aa, Homo sapiens
ADLFYDVEALDLESPK

SEQ ID NO: 997 - protein, 10 aa, Homo sapiens
MTSGDVLSNR

SEQ ID NO: 998 - protein, 11 aa, Homo sapiens
DGHQLKQLLLK

SEQ ID NO: 999 - protein, 10 aa, Homo sapiens
LNNFDFQVQK

SEQ ID NO: 1000 - protein, 10 aa, Homo sapiens
TETLLLQAER

SEQ ID NO: 1001 - protein, 13 aa, Homo sapiens
KVTYTSQEDLVEK

SEQ ID NO: 1002 - protein, 16 aa, Homo sapiens
TMGYQDFADVVCYTQK

SEQ ID NO: 1003 - protein, 10 aa, Homo sapiens
NQVSLTCLVK

SEQ ID NO: 1004 - protein, 10 aa, Homo sapiens
SCDTPPPCPR

SEQ ID NO: 1005 - protein, 21 aa, Homo sapiens
TPEVTCVVVDVSHEDPEVQFK

SEQ ID NO: 1006 - protein, 12 aa, Homo sapiens
WYVDGVQVHNAK

SEQ ID NO: 1007 - protein, 16 aa, Homo sapiens
SGTASVVCLLNNFYPR

SEQ ID NO: 1008 - protein, 20 aa, Homo sapiens
VDNALQSGNSQESVTEQDSK

SEQ ID NO: 1009 - protein, 15 aa, Homo sapiens
SYSCQVTHEGSTVEK

SEQ ID NO: 1010 - protein, 10 aa, Homo sapiens
GSDPVTIFLR

SEQ ID NO: 1011 - protein, 8 aa, Homo sapiens
FDQLNIQR

SEQ ID NO: 1012 - protein, 16 aa, Homo sapiens
RHLDSVLQQLQTEVYR

SEQ ID NO: 1013 - protein, 14 aa, Homo sapiens
GTCEQGPSIVTPPK

SEQ ID NO: 1014 - protein, 8 aa, Homo sapiens
VLVSPLSK

SEQ ID NO: 1015 - protein, 10 aa, Homo sapiens
LTINQLLAER

SEQ ID NO: 1016 - protein, 11 aa, Homo sapiens
ETLFSVMPGLK

SEQ ID NO: 1017 - protein, 13 aa, Homo sapiens
AEAQAQYSAAVAK

SEQ ID NO: 1018 - protein, 9 aa, Homo sapiens
AQYEDIAQK

SEQ ID NO: 1019 - protein, 8 aa, Homo sapiens
YFIDFVAR

SEQ ID NO: 1020 - protein, 9 aa, Homo sapiens
KYFIDFVAR

SEQ ID NO: 1021 - protein, 9 aa, Homo sapiens
RPPGFSPFR

SEQ ID NO: 1022 - protein, 8 aa, Homo sapiens
DFVQPPTK

SEQ ID NO: 1023 - protein, 10 aa, Homo sapiens
VAAGAFQGLR

SEQ ID NO: 1024 - protein, 7 aa, Homo sapiens
GPLQLER

SEQ ID NO: 1025 - protein, 8 aa, Homo sapiens
LIVQVPPK

SEQ ID NO: 1026 - protein, 15 aa, Homo sapiens
DLADELALVDVIEDK

SEQ ID NO: 1027 - protein, 13 aa, Homo sapiens
AGGGDAEDAPSLL

SEQ ID NO: 1028 - protein, 12 aa, Homo sapiens
CGVALSTPGGAR

SEQ ID NO: 1029 - protein, 10 aa, Homo sapiens
AVDTWSWGER

SEQ ID NO: 1030 - protein, 9 aa, Homo sapiens
KSQLVYQSR

SEQ ID NO: 1031 - protein, 19 aa, Homo sapiens
LQSGTHCLWTDQLLQGSEK

SEQ ID NO: 1032 - protein, 15 aa, Homo sapiens
EVDSGNDIYGNPIKR

SEQ ID NO: 1033 - protein, 19 aa, Homo sapiens
RLTVGAAQVPAQLLVGALR

SEQ ID NO: 1034 - protein, 18 aa, Homo sapiens
VLSIAQAHSPAFSCEQVR

SEQ ID NO: 1035 - protein, 10 aa, Homo sapiens
LKELTLEDLK

SEQ ID NO: 1036 - protein, 7 aa, Homo sapiens
QYADTVK

SEQ ID NO: 1037 - protein, 8 aa, Homo sapiens
KIAENKAK

SEQ ID NO: 1038 - protein, 9 aa, Homo sapiens
YEAAVPDPR

SEQ ID NO: 1039 - protein, 22 aa, Homo sapiens
EMLDQSNQWGGTALVVPAFEIR

SEQ ID NO: 1040 - protein, 12 aa, Homo sapiens
NEEALDTESSER

SEQ ID NO: 1041 - protein, 9 aa, Homo sapiens
SQLEGALSK

SEQ ID NO: 1042 - protein, 12 aa, Homo sapiens
GLGEISAASEFK

SEQ ID NO: 1043 - protein, 18 aa, Homo sapiens
CVVTGEDGSESEATVNVK

SEQ ID NO: 1044 - protein, 12 aa, Homo sapiens
LEFPGAEGQWTR

SEQ ID NO: 1045 - protein, 10 aa, Homo sapiens
SADYVNLALK

SEQ ID NO: 1046 - protein, 10 aa, Homo sapiens
LSALTLSTVK

SEQ ID NO: 1047 - protein, 15 aa, Homo sapiens
LGDSMANYPQGLDDK

SEQ ID NO: 1048 - protein, 14 aa, Homo sapiens
RNPQELYDVYCFAR

SEQ ID NO: 1049 - protein, 8 aa, Homo sapiens
ILTFQGSK

SEQ ID NO: 1050 - protein, 10 aa, Homo sapiens
LVEAFGGATK

SEQ ID NO: 1051 - protein, 18 aa, Homo sapiens
VAELEHGSSAYSPPDAFK

SEQ ID NO: 1052 - protein, 9 aa, Homo sapiens
FGEGVSSPK

SEQ ID NO: 1053 - protein, 10 aa, Homo sapiens
VHLIVQVSPK

SEQ ID NO: 1054 - protein, 10 aa, Homo sapiens
LESLPAHLPR

SEQ ID NO: 1055 - protein, 10 aa, Homo sapiens
STILYAGNDK

SEQ ID NO: 1056 - protein, 15 aa, Homo sapiens
EFTEAFLGCPAIHPR

SEQ ID NO: 1057 - protein, 13 aa, Homo sapiens
DTNGSQFFITTVK

SEQ ID NO: 1058 - protein, 20 aa, Homo sapiens
GNDISSGTVLSDYVGSGPPK

SEQ ID NO: 1059 - protein, 11 aa, Homo sapiens
VLPGVDALSNI

SEQ ID NO: 1060 - protein, 12 aa, Homo sapiens
TGLELSRDPAGR

SEQ ID NO: 1061 - protein, 12 aa, Homo sapiens
LVLLNAIYLSAK

SEQ ID NO: 1062 - protein, 18 aa, Homo sapiens
SIASLLTQVLLGAGENTK

SEQ ID NO: 1063 - protein, 10 aa, Homo sapiens
LLDSLPSDTR

SEQ ID NO: 1064 - protein, 11 aa, Homo sapiens
LIVHNGYCDGR

SEQ ID NO: 1065 - protein, 11 aa, Homo sapiens
DPNGLPPEAQK

SEQ ID NO: 1066 - protein, 10 aa, Homo sapiens
QRQEELCLAR

SEQ ID NO: 1067 - protein, 19 aa, Homo sapiens
ATTVTGTPCQDWAAQEPHR

SEQ ID NO: 1068 - protein, 8 aa, Homo sapiens
NLDENYCR

SEQ ID NO: 1069 - protein, 11 aa, Homo sapiens
HSIFTPETNPR

SEQ ID NO: 1070 - protein, 14 aa, Homo sapiens
EPEPPGVVGGPGEK

SEQ ID NO: 1071 - protein, 15 aa, Homo sapiens
EIVDSYLPVILDIIK

SEQ ID NO: 1072 - protein, 16 aa, Homo sapiens
YDSVSVFNGAVSDDSR

SEQ ID NO: 1073 - protein, 10 aa, Homo sapiens
FDLEPDTYCR

SEQ ID NO: 1074 - protein, 11 aa, Homo sapiens
VFDLELHPACR

SEQ ID NO: 1075 - protein, 13 aa, Homo sapiens
LENLLLLDLQHNR

SEQ ID NO: 1076 - protein, 18 aa, Homo sapiens
PALGLDDDPDAPAAQLAR

SEQ ID NO: 1077 - protein, 7 aa, Homo sapiens
MATLYSR

SEQ ID NO: 1078 - protein, 17 aa, Homo sapiens
AQGFTEDTIVFLPQTDK

SEQ ID NO: 1079 - protein, 13 aa, Homo sapiens
AQVSVQPNFQQDK

SEQ ID NO: 1080 - protein, 8 aa, Homo sapiens
SLGSYSYR

SEQ ID NO: 1081 - protein, 14 aa, Homo sapiens
LLQPAGSLGSYSYR

SEQ ID NO: 1082 - protein, 12 aa, Homo sapiens
MASGAANVVGPK

SEQ ID NO: 1083 - protein, 9 aa, Homo sapiens
PLNCELSER

SEQ ID NO: 1084 - protein, 9 aa, Homo sapiens
SALAYVDGK

SEQ ID NO: 1085 - protein, 11 aa, Homo sapiens
TETASTVTENK

SEQ ID NO: 1086 - protein, 17 aa, Homo sapiens
GEKPASPAVQPDAALQR

SEQ ID NO: 1087 - protein, 15 aa, Homo sapiens
WYQMGIVSWGEGCDR

SEQ ID NO: 1088 - protein, 19 aa, Homo sapiens
NPDSSTTGPWCYTTDPTVR

SEQ ID NO: 1089 - protein, 17 aa, Homo sapiens
TFGSGEADCGLRPLFEK

SEQ ID NO: 1090 - protein, 11 aa, Homo sapiens
DDFEGQLESDR

SEQ ID NO: 1091 - protein, 13 aa, Homo sapiens
QLITSFLDSSQSR

SEQ ID NO: 1092 - protein, 12 aa, Homo sapiens
TWELEPDGALDR

SEQ ID NO: 1093 - protein, 16 aa, Homo sapiens
EVPDEYEVGSFMEEVR

SEQ ID NO: 1094 - protein, 7 aa, Homo sapiens
IFLHGNR

SEQ ID NO: 1095 - protein, 10 aa, Homo sapiens
LGPGMADICK

SEQ ID NO: 1096 - protein, 17 aa, Homo sapiens
SQEESEEGEEDATSEVD

SEQ ID NO: 1097 - protein, 11 aa, Homo sapiens
ADQTVLTEDEK

SEQ ID NO: 1098 - protein, 10 aa, Homo sapiens
ETGTPIWTSR

SEQ ID NO: 1099 - protein, 13 aa, Homo sapiens
ATIVHQDQAYDDK

SEQ ID NO: 1100 - protein, 12 aa, Homo sapiens
LQSLFDSPDFSK

SEQ ID NO: 1101 - protein, 19 aa, Homo sapiens
IAQLPLTGSMSIIFFLPLK

SEQ ID NO: 1102 - protein, 13 aa, Homo sapiens
CSTSSLLEACTFR

SEQ ID NO: 1103 - protein, 19 aa, Homo sapiens
SDNCEDTPEAGYFAVAVVK

SEQ ID NO: 1104 - protein, 10 aa, Homo sapiens
IQNILTEEPK

SEQ ID NO: 1105 - protein, 11 aa, Homo sapiens
YVYIAELLAHK

SEQ ID NO: 1106 - protein, 13 aa, Homo sapiens
SAGDVDTLAFDGR

SEQ ID NO: 1107 - protein, 8 aa, Homo sapiens
QICSGQLK

SEQ ID NO: 1108 - protein, 9 aa, Homo sapiens
MQLTDFILK

SEQ ID NO: 1109 - protein, 18 aa, Homo sapiens
GSVGSEPQAFDVFPENPR

SEQ ID NO: 1110 - protein, 13 aa, Homo sapiens
SLMSGLEQMEELR

SEQ ID NO: 1111 - protein, 9 aa, Homo sapiens
IYSFGLGGR

SEQ ID NO: 1112 - protein, 10 aa, Homo sapiens
DEDNNLLTEK

SEQ ID NO: 1113 - protein, 26 aa, Homo sapiens
LSENTDFLAPGVSSFTDSNQQESITK

SEQ ID NO: 1114 - protein, 21 aa, Homo sapiens
NHGVDDDGDDDGDDGGTDGPR

SEQ ID NO: 1115 - protein, 13 aa, Homo sapiens
ETAVSTEDDSHHK

SEQ ID NO: 1116 - protein, 9 aa, Homo sapiens
FFEECDPNK

SEQ ID NO: 1117 - protein, 12 aa, Homo sapiens
HVGDLGNVTADK

SEQ ID NO: 1118 - protein, 14 aa, Homo sapiens
TENCAVLSGAANGK

SEQ ID NO: 1119 - protein, 21 aa, Homo sapiens
GGTLSTPQTGSENDALYEYLR

SEQ ID NO: 1120 - protein, 14 aa, Homo sapiens
YLYHGQTLETLGGK

SEQ ID NO: 1121 - protein, 10 aa, Homo sapiens
VEIDTKSYWK

SEQ ID NO: 1122 - protein, 20 aa, Homo sapiens
LGEHNIDVLEGNEQFINAAK

SEQ ID NO: 1123 - protein, 8 aa, Homo sapiens
KCGMKNFR

SEQ ID NO: 1124 - protein, 7 aa, Homo sapiens
YTFELSR

SEQ ID NO: 1125 - protein, 15 aa, Homo sapiens
SPPLYCDSEIDAELK

SEQ ID NO: 1126 - protein, 15 aa, Homo sapiens
AQLPELPDVELPTNK

SEQ ID NO: 1127 - protein, 17 aa, Homo sapiens
STNYGQAPLSLLVSYTK

SEQ ID NO: 1128 - protein, 12 aa, Homo sapiens
FEDGVLDPDYPR

SEQ ID NO: 1129 - protein, 9 aa, Homo sapiens
IYISGMAPR

SEQ ID NO: 1130 - protein, 10 aa, Homo sapiens
TGYYFDGISR

SEQ ID NO: 1131 - protein, 7 aa, Homo sapiens
IGGDLPK